SEQUENCE LISTING (50 sequences)

SEQ ID NO: 1 - DNA, 8812 bp, Artificial Sequence
  Synthetically generated construct
aaatccaagctcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgagcgctagcgcctatatcgctagcgatcgcgagctacgtacacatcatgcatcgcgatcgagcttcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctagatttcacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgaacgttattagttcgccgctcggtgtgtcgtagatactagcccctggggccttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcctagaattaattcaggtaggtcagatttgagtaacaggtctaacaggtctaggaggagcaggaagctcgaaatctctttgccagaatccaacatcatgccatcctccatgcttgtatccagcagctctaagagttcctctagcagtgtatccaagagcctcatgaagtctaacagaaggatcgttaggaagtccaataacagcaacaacagacttgaatccttgagcctccatagacttaagaagatgagtgtaaagagtagatccaagtccaagtctttgatgtctatgagaaacgtaaacagtagactcaacagtccaatcgtaagcgtttctagccttccaaggtccagcgtaagcaattccagcaacaactccctcaacctcagcaacaagccaagggtatctatcttgaagtctctcaagatcatcgatccactcttgaggagtttgaggctcagttctgaagttaacagtagaagtctcaatgtaatggttaacaatatcacaaacagcagccatatcagcagcagtagcaggtctaatctcaacaggtcttctctcaggagacattttgtttagctgtcaaaacaaaaacaaaaatcgaaacatcagaatcaacaaaaatacatcaaccatcaactatacaacaaccaaaacgtcaacaatataatcaaacacagatccactgaaacaaaaccacatatcaccagttgagctatcatatcaaaccacgagacaacaggtatatcaaatctaaggaacatcaccaaccaaatacatcagaatcaactataaccagagcagatacagatcgacatgataaaaaacatgcgaagacgatatcaaaactaaacgctatcaattaatcagaggattatacatcagactcaataggaacaatattgatcgacgagtaaacggatctaaagctagagaatcaaaagcagtataacaacagcaaagaataagcgataatcacagtcaatatagagctaaaactaagaatctaaaccctaaacagctacaataatcataagaagatgaagatcggagacactaaagagagaaaatatctaacctgcaagtaagaatctgaaaggagtcttgcggctacgaaaatgtgagaaatatgagagcgcaccctaatcctggtcgactcgagggtacttatagctacgaggtgtctagggttttcgctttctctttgtggttctacttttactaatttgcccttacgcgttttgggcctttctatttttttggttgtgaatttacccaacaaagaattacaaaaatggatccacaaaattctcatacatttttttcttcaatttgaaatgttaaatagcttataattatgtgttgtttggttaagaaattgtataattgtataaatttttttataaaaaaactctcttgatgatcgaaaaggtgacggaaaaccctagccgtcatgagttggctttgatagatctatggaattaaattaatactagtatataaattgataaatcgaaattacagcctaattaatgggacataaaacatatatttatctggcgccagaattcgaagctaaatgccatggatgtttaaacctaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcagcccttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacagggcgctcttccgctcgccctttggcgcgccggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccgggcgcgcctgaagtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatggggggcgcacctattgacatttgaggggctgtccacaggctgaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaaaagggcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctattccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttagggctaccatggaggcggcggccaatcttgcttgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctctaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagtgggccctaagcttcatttaaatccactgcagtggttccaagaaagagagcaatggtgccactgaacctggtaatgagcctgttgccagcaagagaccgaagagagcagctgcctgttctaacttcaaagagaagtcattggacttatcagaaaaagattcaattatcacaatcaaggaaagtcgggttgaagagaaggaaatagaggctgttaatttgacaaggacgggacctgaagatggtcaaccttgcagaaaaatcatcgatttcatcttacatgatggagatggtaatctgcaaccctttgaaatgtctgaagttgatgacattttcataacagctcttatcatgcccttggatgatgatctggaaaaggataggggaaagggaatatgttgttcggggtttggacgaattgaaaactgggcgatttctggctatgatgaaggtgctgcagtaatttgggtctcaacagaaacatcagattacaaatgtgtgaagccagcaagcagttacagatcttattttgaacactttagtgagaaggcacgtgtctgtgttgaagtctataagaagttagctagatcagttggtggaaatcctcaggtggacttagaagaattaattgctggtgttgtccgttccatccattgcactggtctagaccc

SEQ ID NO: 2 - DNA, 612 bp, Oryza sativa
  misc_feature (0)...(0): N-terminal domain of OsMet1
ttccaagaaagagagcaatggtgccactgaacctggtaatgagcctgttgccagcaagagaccgaagagagcagctgcctgttctaacttcaaagagaagtcattggacttatcagaaaaagattcaattatcacaatcaaggaaagtcgggttgaagagaaggaaatagaggctgttaatttgacaaggacgggacctgaagatggtcaaccttgcagaaaaatcatcgatttcatcttacatgatggagatggtaatctgcaaccctttgaaatgtctgaagttgatgacattttcataacagctcttatcatgcccttggatgatgatctggaaaaggataggggaaagggaatatgttgttcggggtttggacgaattgaaaactgggcgatttctggctatgatgaaggtgctgcagtaatttgggtctcaacagaaacatcagattacaaatgtgtgaagccagcaagcagttacagatcttattttgaacactttagtgagaaggcacgtgtctgtgttgaagtctataagaagttagctagatcagttggtggaaatcctcaggtggacttagaagaattaattgctggtgttgtccgttccat

SEQ ID NO: 3 - DNA, 8862 bp, Artificial Sequence
  Synthetically generated construct
aaatccaagctcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgagcgctagcgcctatatcgctagcgatcgcgagctacgtacacatcatgcatcgcgatcgagcttcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctagatttcacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgaacgttattagttcgccgctcggtgtgtcgtagatactagcccctggggccttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcctagaattaattcaggtaggtcagatttgagtaacaggtctaacaggtctaggaggagcaggaagctcgaaatctctttgccagaatccaacatcatgccatcctccatgcttgtatccagcagctctaagagttcctctagcagtgtatccaagagcctcatgaagtctaacagaaggatcgttaggaagtccaataacagcaacaacagacttgaatccttgagcctccatagacttaagaagatgagtgtaaagagtagatccaagtccaagtctttgatgtctatgagaaacgtaaacagtagactcaacagtccaatcgtaagcgtttctagccttccaaggtccagcgtaagcaattccagcaacaactccctcaacctcagcaacaagccaagggtatctatcttgaagtctctcaagatcatcgatccactcttgaggagtttgaggctcagttctgaagttaacagtagaagtctcaatgtaatggttaacaatatcacaaacagcagccatatcagcagcagtagcaggtctaatctcaacaggtcttctctcaggagacattttgtttagctgtcaaaacaaaaacaaaaatcgaaacatcagaatcaacaaaaatacatcaaccatcaactatacaacaaccaaaacgtcaacaatataatcaaacacagatccactgaaacaaaaccacatatcaccagttgagctatcatatcaaaccacgagacaacaggtatatcaaatctaaggaacatcaccaaccaaatacatcagaatcaactataaccagagcagatacagatcgacatgataaaaaacatgcgaagacgatatcaaaactaaacgctatcaattaatcagaggattatacatcagactcaataggaacaatattgatcgacgagtaaacggatctaaagctagagaatcaaaagcagtataacaacagcaaagaataagcgataatcacagtcaatatagagctaaaactaagaatctaaaccctaaacagctacaataatcataagaagatgaagatcggagacactaaagagagaaaatatctaacctgcaagtaagaatctgaaaggagtcttgcggctacgaaaatgtgagaaatatgagagcgcaccctaatcctggtcgactcgagggtacttatagctacgaggtgtctagggttttcgctttctctttgtggttctacttttactaatttgcccttacgcgttttgggcctttctatttttttggttgtgaatttacccaacaaagaattacaaaaatggatccacaaaattctcatacatttttttcttcaatttgaaatgttaaatagcttataattatgtgttgtttggttaagaaattgtataattgtataaatttttttataaaaaaactctcttgatgatcgaaaaggtgacggaaaaccctagccgtcatgagttggctttgatagatctatggaattaaattaatactagtatataaattgataaatcgaaattacagcctaattaatgggacataaaacatatatttatctggcgccagaattcgaagctaaatgccatggatgtttaaacctaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcagcccttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacagggcgctcttccgctcgccctttggcgcgccggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccgggcgcgcctgaagtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatggggggcgcacctattgacatttgaggggctgtccacaggctgaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaaaagggcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctattccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttagggctaccatggaggcggcggccaatcttgcttgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctctaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagtgggccctaagcttcatttaaatccactgcagtggttgagctaggtggttcagacaaaccaaaggatgggcaatcagagaactgtcttgcaacacttgacatttttgctggttgtggaggtttatctgaaggattgcagcgatcaggattgtcacttactaaatgggctattgaatatgaagaacctgctggggatgcatttggtgaaaaccatccagaagctgcagtatttgtcgaaaactgcaatgtgattctgaaggcaattatggacaagtgtggtgattctgatgattgcatctccacttctgaggctgctgaacgagcagctaaactttctgaggacaagattaagaatctgcccgtgcctggcgaagtagaattcataaatggtggccctccgtgtcagggtttttctgggatgaacagattcaatcaaagtccctggagcaaagtccagtgcgagatgatcttagcattcctgtcatttgcggagtatttccgtcctagattctttctcttagaaaatgttaggaactttgtctcgttcaacaaaggacagaccttcagattgacactggcatcactcctggagatgggataccaggtccgatttggaattttagaggcaggggcttatggtgttgcgcagtccaggaaaagggcattcatttgggccgctgcacctggagagactcttccattgcactggtctagaccc

SEQ ID NO: 4 - DNA, 662 bp, Oryza sativa
  misc_feature (0)...(0): C-terminal domain of OsMET1
ttgagctaggtggttcagacaaaccaaaggatgggcaatcagagaactgtcttgcaacacttgacatttttgctggttgtggaggtttatctgaaggattgcagcgatcaggattgtcacttactaaatgggctattgaatatgaagaacctgctggggatgcatttggtgaaaaccatccagaagctgcagtatttgtcgaaaactgcaatgtgattctgaaggcaattatggacaagtgtggtgattctgatgattgcatctccacttctgaggctgctgaacgagcagctaaactttctgaggacaagattaagaatctgcccgtgcctggcgaagtagaattcataaatggtggccctccgtgtcagggtttttctgggatgaacagattcaatcaaagtccctggagcaaagtccagtgcgagatgatcttagcattcctgtcatttgcggagtatttccgtcctagattctttctcttagaaaatgttaggaactttgtctcgttcaacaaaggacagaccttcagattgacactggcatcactcctggagatgggataccaggtccgatttggaattttagaggcaggggcttatggtgttgcgcagtccaggaaaagggcattcatttgggccgctgcacctggagagactctt

SEQ ID NO: 5 - DNA, 1187 bp, Artificial Sequence
  Synthetically generated
ggatcccccgggctgcaggaattcgatatcaagcttatcgatgagtttctcaaagtttggaccttgattatcttgtttggagatgttcaaatcgttatatccaaatagtgaacttctaattttcttttttgataatgtgacttatttggaaaagtattccaaagtattcaaataaaccctttaaaaatccattaaatacattttaaataagtaaaatgctctcaacgaagagatatcatggtaaataacaacagtgagaggataaaatgttaaatcaatttatttacaacttcaaataggcggacatcaaacctacttagcacactttctattttcaaattggttatggtttgtctattagttgttgcatctatgttttttaattcttatatcggtgatcttgattttgttttggtgtatctaaaatctattttagttaaagtgcaagaaaataaaataaaaacttaaggtaagagatgaaagtaagctttaaataaaacagagcacttctatggtcgattatagagccaagttcgttcctccattttggcttaatgcaatattacaagtaaatcttataaaactttccataagtatcgtattacccatggatactatgatatataaactctcggaggtgtagtccagaagaaatgatccatatttgcatacagtaaacttgatggaaaaaatatgtggtactgttggaattgtagctattgagtatcaaatttgagaaaaaggtaaaaaaatatgtaaaatttgggtggaagaaaagaattacataaaattgagaaatgtatgtaattgacaaaataatgttttcaaaacataaaaacgtgataccatttaaatccaaaccttatatcatttaaccatttttagtaaaactaatagtaatgaatggtcaataatataagattacatattaaataattactactttcagaaaatttcaatcaaatctataatattcctttgaaaaaaaagaaagacaaataggtaaacttcgatcgtatcaatcaaagaatatatttatttttcatcgtaacgtttaattctaagtcctattaaaaaacgttaaatttgatttttcttaccatttttttctaaaaggtgagttgtgtgttgtgtcaggtccaaaataaaagtttgtcgtgaggtcaaaatctacggttacaggatcc

SEQ ID NO: 6 - DNA, 1019 bp, Arabidopsis thaliana
  misc_feature (0)...(0): YP0102
gaggtcagtgaagtcgattgggatttggttgataacgttttactcgactaattatatacttcagaaggatagtaatagaataccaaaataattaaatgattggttagtgccttagtggagactttttaaccgattctaatagactaatgatgtagctaagcattatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcatatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaaca

SEQ ID NO: 7 - DNA, 1023 bp, Arabidopsis thaliana
  misc_feature (0)...(0): YP0117
aagaagtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttttaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactgatagtcactttggaactgagtagatatttgcatcttgagttaccatggactcaaaagtccaaaaagagaccccgagtgaaaatgctaccaacttaataacaaagaagaatttacagcggtcaaaaagtatctataaatggttacacaacagtagtcataagca

SEQ ID NO: 8 - DNA, 1005 bp, Arabidopsis thaliana
  misc_feature (0)...(0): YP0180
ttattgttgaaacggatggtatccagattcatagagttatacgttgttgacctcgtacaggatgaattcattatcttcttcttcttttgcagcatggcaggtgatcgatgggtatgacttgtgatgatagccatgtccaccaaatcagccaagaaaagatcaagacctcggctgcttacgttctgttctataaacgccttgtagactaaagaaactgaagcggaaaagacaagaaagaggtatttgcatttttgccgggtttggcttatttaaaaacatcattggcttgattctaattcactacaagatcaagatgaaagcagctctgcgttgaggctaatttacagaagagagagagagagttgggaagaagagcaaaagaccgagaggacatgttgcggggaatttattttattcttacaaaaattggtatctgattattttattaaccatattcaattagagaatagaagaatagagaaaagcccttttgtgggatatggttctaaattgttgtttagttcttgtgtgtcagttttggctctcgtcgaccaaagaagattaaagaaacctctaccttattttaactcaattcttttgtttttgcaatgtcctttgctttccaaaattgttagtcttacttttcactactttgatagacattgcctttgcgtttccctgattaataagccagagtacttaaatcaaaattgactgttttgtgcatcctgcatcacgtttccaatcagaaccatagtgttgtcgttgtgtcattatccgaatttaagtggagacattggtaagttatttataaactaattacaatctatttttctaattatttcaaataacatatttaagctctgtagcttccactagacggtgaagatttgaagtgagagctctctttgcattgctcacccaccaatggatctacctacccttcttcttcttctccgccttttaaaccctaaaagtttctctttccttcaacaacgccacaat

SEQ ID NO: 9 - DNA, 1002 bp, Arabidopsis thaliana
  misc_feature (0)...(0): YP0110
ggaacgttagctgctatagcaaagcatggaatggcaatgtcagatccggaacctgaaataaacgtgtatcagatcgcttcttcggcgataaacccgctggttttcgaagacttagcggagcttctttataaccactacaaaacatctccatgcatggactctaaaggtgatcctattatggtgcgtttgatgaaacttttcaattccgttgatgatttctcggatcatttgtggagagatgctcaagaacggagtgggttgatgagtggtatgagttcagtggatagtaagatgatgcagaagctaaagtttatatgcaagaaatctgttgaacaagccaaacaccttgctactatttatgagccatacactttctatggtggaaggtaagacagaactcattaacattctaattcttagagcagacaaaaccggtacccgcaaagttttcatcttttttttttggtttcttttacagatttgataacagcaatacacagagattaatggagaatatgtcagaggacgagaagagagaatttggatttgatgttggaagcattaactggacggactacattacaaacgttcacattcccggtttaagaaggcatgtcttgaaaggaagagcttaactttgaatctcactaaaccagaccaaacagaatcgatcccttcttttatctttttatctttttcttttttcattacgtgtaatcgtgttgtgtctaatatatcagtttgatttgtaataatttgaaaaaaaacggaaatgttgttatctttaagtttgcccaaaatctatagtcatgttcgattcaagacaaagtttaaagttacaacctgtaaaaatattaatagtctctgatgtaaacgtatcttaaacaaaattattaaatgttgaagttagtaacatacaattattaatgaataaatgtttaatcaattaaatgtcatttagtgattgtcctataaaatctcttgttttcttgttttatatta

SEQ ID NO: 10 - DNA, 1000 bp, Arabidopsis thaliana
  misc_feature (0)...(0): YP0039
ccgttcgagtatttgaaaatttcgggtacacccgcctaaataggcggaccttatctagtatatatatacatttgaactatattgtttactttttagttgatttaggctatgtatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacga

SEQ ID NO: 11 - DNA, 1000 bp, Arabidopsis thaliana
  misc_feature (0)...(0): YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaataaaagtccaaaatatctaaacactaatctaccacgtctattacacacacacacacacacttgatcttaatttattttcaagattcaagaaaatacccattccattaccacaacttgaccacacgcctatatataaaacataaaagccctttcccc

SEQ ID NO: 12 - DNA, 1000 bp, Arabidopsis thaliana
  misc_feature (0)...(0): YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 13 - DNA, 1000 bp, Arabidopsis thaliana
  misc_feature (0)...(0): YP0137
tggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaacttatgaatctttacttaatatatacaacatatctttaccttgcggcggagaagatcggcgagagaagcaccccagccaccgtcactaaaggattcttcagtgatggaatcaccaaagagaaaaatcttccgtctcatcatcttccacacaatcttcttgagaaaatctgagagataagataggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttaaattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 14 - DNA, 985 bp, Arabidopsis thaliana
  misc_feature (0)...(0): YP0212
tacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatggggcagt

SEQ ID NO: 15 - DNA, 2066 bp, Arabidopsis thaliana
  misc_feature (0)...(0): DME promoter
tggtgcaattagaaacgaacatagtcgtaaaatacgagttcggtgttatacctttatttacgttaaaaaaatacgagaattttgtgtcaaatttcaaattaatttcatgaatatatggaaattattagatactctagcgaaaatagtgattatgagcgttttacaaaaatacgattttagcattgaacttcctttatgtaattcggtcaaatgttggcatgaagaagcaagtttgcaacattaaatttcatttaaaaatcgtgttgacatactttaaaatctaaatataggaagaagaccaaaacattaaatttagtaagattctaatgaacatttataagttataacttataaccaacaaaagttgggtttagcgttgttgctttatctgaaaacttgcaaactaaaccattttaataggactaatgacaattaacaacaaaatacacttaagcaacaacgtcctcgtgaatataatttgggcctcaggcccatattgctaacgccaactgatatttcactttattccttcttcatctcaccacactctctctctatctctatctctaacggcatagctgactcagtgttctccggcattgactcgcctgagaatcagaaagcttagatcggtgagcttttagctccattttctgtttatttacatattatttcctttttttctctctcccttttttatctggaatttgttctgctaaattttccagctgttacattttccgatcacgagaagaatcactgggtttttatgttaatcaatacatgttcctgttttctgatcataaatctcagctattaacacctgattttgattctgcgtaataaaaacctctgatttgcttttatcttcactttccccataaacattgcttactttattcgctcttcttttaccgtttccagctaaaaaattcttcgctattcaatgtgtttctcgttttgttgatgagaaaaatatctgacaaaaaatcatttattgcattttatggtgcagattcttagttaatgtcgccttctctaaccaagtcagattaaaaaggagtgttcgtccatgttgctttgttttggtgtttggagagagttttcggagagttaggtgagtgttatttggggtgaggtagtgataaggtttgaagggggagtgattcatcaagtgtgttatgaattcgagggctgatccgggggatagatattttcgagttcctttggagaatcaaactcaacaagagttcatgggttcttggattccatttacacccaaaaaacctagatcaagtctgatggtagatgagagagtgataaaccaggatctaaatgggtttccaggtggtgaatttgtagacaggggattctgcaacactggtgtggatcataatggggtttttgatcatggtgctcatcagggcgttaccaacttaagtatgatgatcaatagcttagcgggatcacatgcacaagcttggagtaatagtgagagagatcttttgggcaggagtgaggtgacttctcctttagcaccagttatcagaaacaccaccggtaatgtagagccggtcaatggaaattttacttcagatgtgggtatggtaaatggtcctttcacccagagtggcacttctcaagctggctataatgagtttgaattggatgacttgttgaatcctgatcagatgcccttctccttcacaagcttgctgagtggtggggatagcttattcaaggttcgtcaatgtgagtgatcaaatctattttcagtttttttttttccctttcttccgttcttgcagtacttagagtagaacatgaattagaatatcttaagaaagtcatggttttgaacagatggacctccagcgtgtaacaagcctctttacaatttgaattcaccaattagaagagaagcagttgggtcagtctgtgaaagttcgtttcaatatgtaccgtcaacgcccagtctgttcagaacaggtgaaaagactggattccttgaacagatagttacaactactggacatgaaatcccagagccgaaatctgacaaaagt

SEQ ID NO: 16 - DNA, 1912 bp, Artificial Sequence
  Synthetically generated
gcgtacatggaagttttatgagattgttttagcgttacattattgttctcatgggttttgttgaaccgtgctatagaaccagaacgaagcaatagtcacgtaggataaaccaaatcaccttatctattaggtgtatatggaagttttatgagattgttttagcgttacgttattgttctcatggtttttgctgaaccgtgttatagaagaacccggaacgaagcaatagtcacgtaggataaaccaaatcaccttatctattaggagtatatggaagttttatgagattgttttagcgttacattattgttctcatggtttttgctgaaccgtgttatagaacctagaacgaaacaatagtcacataggataaacaaaatcaccttatctattaggtgtatatggaagttttatgagattgttttagcgctacgttattgttctcatggtttttgttgaaccatgttatagagcccgaaacgaaacaatagtcacataggataaatccaaatcatcttatctattaggtgtatatggaagttttatgagactgttttagctttacgttattgttctcatagtttctgtagaaaccgtaacctgaaacaaagcaaatggttacataggacaaaccaaatcacacaaacttcactaattggtaagcttggtaggctcgcaggaacgaaaacacaactaattggtaaaataaatcgcatttgacatatctagctaatccgattaatcttatactctcatcatctaatttttagctgaccaccagcttccaaattttgaaatttgaagctttgattataggatttatttttcatctaagtttactttccggtcttcgatttcaaattgataatgatacaaatataaaaacttttacttttatttgaaagccaaatgaaaaataccctgaaacgaagaaaaagtcatttaagacaaacttagagataccccgatgtgtatgatcaaaatggggtctgatacactgctgatcagttcccacattgattttggtgtgatattccgttccataatcgtctttaaaaaacaaaagagggaaaaaaacaaaacactatgcaaccgtgcaaatgaaagcatcgtcaaatgattaaaaacgtcaaaccaattcaatcaaccccaaactccaaaccaactttttttttctcttttcttttttttctttttgtcgatcttgagcgaagcaatcctccaaagtccaaaccaccaatcgaagcaagaacacaaaaacaaaaaacagcaccagcgaattcggtgccgcccatcggttatggctctcgccccacacatcttgcgttccttctcgcagcaaacatttcccaaatctcaaaaaaaaaaagaaagaaaagaaaaaccaaaagaggaggatgataccgtgatgacaccatgcaaggcagttcgtcacatgatctggttcgctccaaaaagctgatagtaaaaatcatcccaaaatatctcctcggagaaaaattcttaccacaccgtccctctcctgttcatccctgttcgtggccgaatcttttgtttttaccgaggaatcttttgattagtggttgtagtgacatcatggacagaagaggaggttggtaattaggcggggtaaaaaaggaccgaggcgacgcgagagctcgtctcctccactcctcgtcctcgtcctcctcctcctcttcctccatttttttttcttttctttttatttgattacgccgtcgctgtcgagtagcgcgtcagctgcatccgcggttataagtagcggccaccacccaccacccccggcttcctctcccactgcgccctccgcgtgagcggcagcaagtgttcactgcgttcttcttctcgatttatctttcttggtttcttgatctgtagcttattagcggcc

SEQ ID NO: 17 - DNA, 1946 bp, Artificial Sequence
  Synthetically generated
ccctgattcttctgatggaactaggggaggctgtgtggccatttttcccgttggagggtttcgtctagatctgtcgggtgtgggacatgcggattgcaggtgctgtcggttgtgttggcggcggcgggtcctgccgggatagttggccgccgacggccgcttggctgttgggttgcacggtgtgtgctggctggtagcgaggatggttttagggtgttgggcgaaagctctgtccgactcatagccggcctgacggcgatgaacgtccttggacatcatgcaatgcccctcctggaggcgtcgtcgcaagagcatctccagtagagaccctaaatacaattcctaaacagtttttaggtgctaaggacaaaaaataaactccagcaaaacccatactacaggtcctaaaataggaaggacctcaaatacccctccgcagtccctaggcctgggggctgtagaccgaggccctatcgccgtttttctacgcgggaggaaatttcctgacgtgtggtgtctgtcttccctcccgcggaatcgctgccacggcgccgatcttcgccagctcgctggttccgccgctcgtggccgacggtgcgaccatccagtacctccaccggccactgcttgtcgtccgcgtgcccgcttgcttgttttttcgtggtccttgatcagttcgcacactgatgcactatatggtagacaagaatgttctgaaattcatgaccatcagaaacatgttctaaacaatcctgctctcgattggtttatggctaactgtggttctaaacgatcatggcataaaaattattgttctgttcctttaaagtttgtggtgcttggtaggttgagacaattaggctgcttgcaattatgcagtagttccttcaaagattattctgcagtgttgttcttttgtgtcagttgtgagttgaagtttaacttcaaggttttttttttctaggaggatttaagctctttctgaagtttctcagatagattagattggaaaaggtatagagttaattttatctattgattatagttcttatttaattgaactacgtagtgtcttgaatacttgccggtaggatttcactcccatgtttgagaattttgaatttgaattatggtatttaaaattatggatttgaatacaattgaattctatacattagaaatattcgtatttgaattattactatgttaaactaggtgtaagcatagagtataatcagaaatacaagagaaaaagaaatgggggctaagaaatagggtctgctggtagagttggaggtaatttttgaattcttagaaaatagggacagccctcattcaacctttgaggactctaaaatagggactactgctggagatgctctaacaccctgttcccccttgctgctgggtgaaaaccctttccagtctcctgtttatgcgatggtggcgtcctttccgacgtcgtcaccttcttcaaggcatcgtttttggagaaaccctgcaaccagtccccctgctttcccatccttctcccctattccatcccctcctcctccccttttcttctgtcaagggctcctatgcttggaaactctcatgtatctcttctctgtaatatattcaggtggggaaatgttggatttttattgattggaatactgtattgggtcatctcggtgacaccaaagctgtactttggtggagtagcaatctttgcccttattgaccggataggattttggttaaatttatctacgtttttgtttgcggttcatcttttttcctaccagtcttatacaagatggtacagtttagcaactgattgttacattgcaatatataaatcgaagtgatagaagccacctcaagtaaatctaactattgttcataattcaaaggtcaagaccaatttctcagttcctgcgactgcgcgaaaaaacaaaacc

SEQ ID NO: 18 - DNA, 1951 bp, Artificial Sequence
  Synthetically generated
cgagatccaccgatggtttacgcgtacgccgacggctcacacatcccccggtgcccaacagaaaccacacaccacccgcacgaaaaaaaccgaaccgcacgtgcgcgcgcgctccacgcacaccccaaacagacggcacggcgggagcgcgcgcgcgcacgcgagccgaggagaaaacaaacgggggaaacaagctggaaaagcaaaaggggaaaagaacggagcggaggcttcacccacggccaccgcgacgcgccaccagcgtgcggtgcaatgcaacgtacgccaagccgaaacggcaggcagcatcgcgcacgcacgcacacacaggccacagcacacgcgagcgacgtacgcgagtgcatgcagatgcatgcgcggggctcgcgcgagaccggccgatgggttcgcttctcttctctctcccgtcccgttgcgtcgtcatagacaaaagtcggttttgcttttggttttttggctctgaggcactgacgtgcgggccagcgtacgcctgcgtgccccgcatgtcatcgtcgacaccggccggggaccgggtaaaatgtgttgcgggagggagagggggagagagagatcgcgcgggcttcacgcaacggcgctacaaatagccacccacaccaccaccccctctctcaccattccttcagttctttgtctatctcaagacacaaataactgcagtctctctctctctctctctctctctctctctctctctgcttcacttctctgcttgtgttgttctgttgttcatcaggaagaacatctgcaagttatacatatatgtttataattctttgtttcccctcttattcagatcgatcacatgcatctttcattgctcgtttttccttacaagtagtctcatacatgctaatttctgtaaggtgttgggctggaaattaattaattaattaattgacttgccaagatccatatatatgtcctgatattaaatcttcgttcgttatgtttggttaggctgatcaatgttattctagagtctagagaaacacacccaggggttttccaactagctccacaagatggtgggctagctgacctagatttgaagtctcactccttataattattttatattagatcattttctaatattcgtgtctttttttattctagagtctagatcttgtgttcaactctcgttaaatcatgtctctcgccactggagaaacagatcaggagggtttattttgggtataggtcaaagctaagattgaaattcacaaatagtaaaatcagaatccaaccaattttagtagccgagttggtcaaaggaaaatgtatatagctagatttattgttttggcaaaaaaaaatctgaatatgcaaaatacttgtatatctttgtattaagaagatgaaaataagtagcagaaaattaaaaaatggattatatttcctgggctaaaagaattgttgatttggcacaattaaattcagtgtcaaggttttgtgcaagaattcagtgtgaaggaatagattctcttcaaaacaatttaatcattcatctgatctgctcaaagctctgtgcatctccgggtgcaacggccaggatatttattgtgcagtaaaaaaatgtcatatcccctagccacccaagaaactgctccttaagtccttataagcacatatggcattgtaatatatatgtttgagttttagcgacaatttttttaaaaacttttggtcctttttatgaacgttttaagtttcactgtctttttttttcgaattttaaatgtagcttcaaattctaatccccaatccaaattgtaataaacttcaattctcctaattaacatcttaattcatttatttgaaaaccagttcaaattcttttaggctcaccaaaccttaaacaattcaattcagtgcagagatcttccacagcaacagctagacaaccacc

SEQ ID NO: 19 - DNA, 1836 bp, Artificial Sequence
  Synthetically generated
gcctctcgaccacgagtttagcacttgtgcaacatatatgcgtgcgatgaacatctactgatgcgccatgcgaattttagcgttcgttcatgacgcttccaacggcacagaggctgagcagcagcatgcatgcatggctcttgtgaaaacaaaaaaggttactggtaaatgacatgctgctgtagctagctagcagaatgcaaggcccatgcatatgcaatgctatgcgacaagtacagtaccagcatgtatggtagccagctaactaatctatcagcagaggcagcaagctcgtgcatggtgtgatgcacttctctccagtaatctagtggtaattttcacccaaagcgttgctcatatggacagtaattagtaatattaccaaggttcacaatcccgttacctgaccaaatactactcacgaatggtatctctggttttcgttaaaaccgttggtaaaccagcaaaaatagacaaaatttgtcaaaattttaaattttagttttttttttttaacttagccgggaaaccttgaagtttgtgctgtcgagctgtcctgggaaggacggttttggttgggattgtgaaccctggttactgcacttcatttttgaacagatattagtgcaacagacaaatgccaacgcatttttttctgtttaccggcaagctgaagcttttacgatccccatacagccgttgctgcaaacctgccaagaaagagcagcagaaacaggtgtcattttgtggtggaaagccaagtaaagtaaacagaagatggaagatagtgaggaccagggagtgaggcaggggacacatggcccacgcctccctgcacattttcgtgtataaatacaggtggatgcatcgctctcccagcatccatcggttctctgctctgttcatccatagagtttcctcctcttctcctttagtgcaaggtagagaagagcatgtgtgtgtgtgtgtgtgtgtgaactgtgaagtgcagagtgcttctgtagttctgtgttatgtccatagtgatcttgttaggattgttgctatggatgcatgatgttatggttgatctctgaattacagtagggacttttctgagatctctggattagtggggggtgctaaatttttttctggttgcatcagcttgggtttctggtattggtgtgggttcttgctctgaattttggttcagaatgtcgatttgtttgtgtttgttctctgaagttgagagtagctatgatccatccagcacagaactgcaggtcctgcctgccggctgcatatacaggacatgccattttgcaagctctgggcttatggtttctcttttggagttcttcttcttgcatgatctgtgttctctaacaaaggaagcaagatttagcaactttattcagagacaagaaaaggatctggcaaccttttgtttctgttttatcctactcgtaaagattgttatttaagcaaaaatttcccaaaagttttaaatataatttccatgatgtgccactctcatgtccttgaacctggcactcattatgggctcctcagaagtgctgtagctaatgtcactaatcttttgtatctttgttcatagtcttgtattttatgatgcttatccctttgtgctttccatgtttgatgtccaaatgtcatggcaatgtttttgacttctagtaggggttttagtacctttttgttagataagtacatccaaattctgtttatttattcaaaaatcattctgtttattcactgaaaacatttgtccattcaatggactcataaactgtctgtgtttttcaggcttgaggatccatctagaagatagca

SEQ ID NO: 20 - DNA, 1895 bp, Artificial Sequence
  Synthetically generated
acaagcctatttcacccttacaacaattcggaagaatatagatgggttttaaacatttgataatatttgctccccactcagatttggttactcgaaattgtacaagacctgacattcgtcatctggacactctagtagataaacgtgctggctgatgctagataaacagatgtaaagatgaccacttcaccatcaaccgtaaaaccggacgaagatcaccaaaaattgatactttggagcaacgataggcagcttcgattcagatagcacaatacttaaaagaccacatactagcatcgaattgatacatctcccctccaaatgaggctccaaaaactatccattgtttgatacagcaagcaataggatgtgagaaactgagattggcatttgtatttcactactctcatctgatgagacatgactaggctgtaactgaagctgaatctaaagaggaagattagtgtgggattgcagacaaaactgctactacttccttcctgcactgcaagaagaagaaatctgtatccagtctgtgttgaaccccattaaagcacacacagcagcttcgattcagacagcaaaagaaacattctgatagatagcatcaaattgatactagtatttcgtttgtgtcaaaaaaactctcgatatgtcgtaatcaaagctcgaaaatcccatttgtttgatacagcagcaacagcaagaaaggaacccctactccgatccagccactgaaacagtactaatgaatccggattcgcgcattcatcctatctgatgtgatgaaaagaagctagagtataagaatctaatctgggagaaggttgaggtcagtcgtcgaaggcggatgaggggtcggcgaggtgggcgaagcgggcggcggaggcggaggagaggaggaggaacttgcggacgcaggacctgacgcagtcctcctccttcttgccgagggttcgccggtagaaggtggtgacgcagtcggagaagcaccggtgcgacacccagttgtacagccgtatcctgcgtgcacaaaaatccatccatcgctactccactctctctgcgaggaggaagggaaggaaagtaagagattaaacgtacgcgtctcgggtctggagcttgtcggcgacggcctccatgcgcgccttgtcctcctcctcctccccgccgccggcggccatggcggcggcggcgtccatgctcttcttcagtagcagcacaagaagaagaagaagaaggagaaggagaaggagaagcgtagcccaagccctaaggccctttagtatagttgaagtggtgagatgggccgtggtgggccttcggtaattgagcccatgggctcaaccccgaaaatgccagtgggctaggtgaggtaaaccgtgcacgtgacgctttcagtttcttttcttttctttccttattatatcatcaaaaaaagaaaagaaaaagagaaaaaaaggtatggaagatactgtatagtatacgctagcagcataagctccgtccgtataattatttcttgtacgcatatgatgtacagtatgtattttacgagctgtatactaccattgcgttggatttatgctggagctatttgcctatgtagtggagtattctagaaggatgcttgtgcgccgtccattgcctgcagaaacggacggcgcgggtgggtgggccccacagggcggtgactgacgcgtgggccaccacattgggatttggctttgctttgctttcgtgccttgtcagccgctgcccccggccccttcttctccttcttcttcttcttcttcttcttctccctcaccatcaccaacaagagagaggaggagtggattcatcgatcgagaagtcgaggtagtacatacgttggattggattggaggaggaga

SEQ ID NO: 21 - DNA, 1773 bp, Artificial Sequence
  Synthetically generated
tgtgcctgtgccgctttacagccaagcccattagcaaggctcaaagatgggctagtttttctcggcccaagccgtctgttgaacagcgtggagaaggccacacggcccacgtgcatacgcaggccgcgcactggatttcaagatgggctgcgcgaggtggacggcccagattgctacggccttctacggcgtcacgttttttcgtggtgcggctggtgcccgtgcttcgcgtacacgacagtgtacacgctgcactgcactccaaagaaatccgccgaaagtgcagttatacgtagcgacaatctgcaatacgtaccaacagccgaaagcatatatggacaagcagccacgcaagccatcagcacaacccacacgaagagcaggttttttttttcgaatcaagccatacggtagtgcgacgtttctattgatatagcaggaaaaaaaatacaaatctatagcattgagagactagttaggagaagaaaaagacggccacaccacatgcctacatctgatcctgctactgaaaacaaaacaagcacacgacacctagaaggaatggttcacactaagagaagttttaacaaaggagagaggtggttgttggaatcaacatgtaattccaatagaaaaaagaacttgattagttgtagtaatccgtaagtaaacagaatcatatagataatggtacaagcctgacccagttgttgatattttttttaatctccctgtcttgcacgtgcggtatagatgctaatgtgatgtggcagcaccgacgtcacacctgtgacatctggccatatgtctacagctaatgctgtgttttgttcaatttttattaaaggcaaataaatatctatatctacggttgtgcctataccaattgaagttatgtcatatgaggcgttttcgtgctatctactgatgaaatttacctctcgtacatcagaaccgtgcaatatcattacttatgtcagtgtaacgggataaattggtagagtttttgagagtggaagcttcctgttttttcaaaatttggtaagatagcaataacaataatgagtttggtttgttgtcctattaaaatttggtaatgccaaaatttagtagggttaaaaataacaacaaagtaaatattccttagtttaaattgttttagttgaaggttaaacattaccaaaaattggtaggttaaaaatgttaataaaaaaagcaaagcccttagtttaaattgtttcagttgaatgttcaacattgctcacaaaatgttctcttaaatagtactttattattacaaagagcatctgaatctgtattaaaaaagtacaaaaaaaaacattctgaatctagaaagggaaaatatctagaagcgactgcacgcggcccccacgaaaagcccatgcacgtgggccccatcccgaaaaaagagcaacagcctcaccgcctacctgcatgtgcaagtggacggtgcgcggctgcgcgccgcaacgcgacgcccccccccccccccaccccaccacccaccggccccacacgtcagctatacagtgggacccacccctccggccccacatgtcagcaagacagtgatacctcctcccccgcctcctcgcgcggcgcgcaacgcacacgcttccccttcatctcagtcgcgcggactcctcagtcctcacactccccacgaactcgaatccccaactataaataatccaccggaaaattcacaattcgatcgcctctctcgatcggagatttcgcaatttctccgcc

SEQ ID NO: 22 - DNA, 981 bp, Arabidopsis thaliana
  misc_feature (0)...(0): 5′-UTR YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 23 - DNA, 1894 bp, Artificial Sequence
  Synthetically generated
tccgcttgcttggagaattttgcgcgttcacaccggcagaactattatttttagcttaatcaaaccggccatgtgatccctgattattttctgtttttttaactcaccaaatttatttcaaattagaaacatattacatggttaaccttacatttgaatgaactaaagcaatcttcaaatctttcgcaaagcatcttttactaggataggctaggtgagatatgttgtgacaaacgtgagctggatcgatgctatagtttgtacacacctttctcatataaagagtgataaaactccaaggaaaaacagattagcacttttttggggccatcctaatgcaagcaagcaaggcttatatggcctgtgcttttttgctttaataagccttttagtccccttccctagtctcatgaagttcatggcaccaaacacctcaacaagtggcaaatgatgaaatgatgtaaatgcacaactactttattttgggctggacgtgttggttctcaactgaacctgcaccgctatcagacagtgtacataacgcaatcgctgagcaaaggaaacagaaaggctactgcccagcgccattttatttggccatttctgctgcaaaagctctctttatttgtttctgaatatttgaatgccaatttggcgacaccaatttctagagagtttccgtggtggcaagacaacctggtacttattgtatagtgctttccttttcgagttgattttccatttgcatttgcaaagatttatataacaaatttgagtataaagaatacatcagtgatgaagtggcgtgactggctcaaatcgagctaagagagatcactcgagcaataatgaacagtgaatcagaataatggatacgttactgtccagtacattgctactgatccttgatgcgtgtgttttgtggtgataagtttgagccgtaaaagcagtggtcgaagctaaacaaaacaacaccatcaaaccaattttggagttttatctgggatattatgcgtggtagtggtattcttggatgcctttggtgacataatttgttgttgaccccaactttttttaaggacaaaaatgtttgtgtcaacactagtgttactatgtgcccatgtcatatgtacactgcttaagcggtgagcaccagaaacatacaaccgatgaagcgtacgttgctcacacgagcaaaagtaactttggtgtaaagatatttggctcttctctagtttgttggagcacattacgttgcattttcgacctattataagtcacactaaccattttacattttcatgatctgctcaatttcgtgcacacctcctgtacatgttaatttctctctagtgctaattaacgatgggctctgcacaaactcccctggttttgatacagacaagtccaattttattcccgcttaaaactaacaaagcttgcattttatctataacacgtctaatttcttgtgggcactgcacatattcccctggttttgatacaggcgtatccaaaattcactcacacttaaaagctcaaaaaagctcccattttaatcaccacacgtctaacaaatttcttgttcacatccacagaagaagctatccatgctgtactttacattgcagtattagactttttatactacttttacattacattattagacctttttttaacacaaaaatccacctacccaaccaattttttgccgggctggtcctcctccccccgcatgagccgcccgtgcgatgacgtctcccggtgggtcacaccgtcacacaccgtgctataaataggggggcttggcctctccgccatgagcaccacacttcaccagcttcgctttgcacaaagcctcagtgcctcactgcacttgcaccggtcacta

SEQ ID NO: 24 - DNA, 1854 bp, Artificial Sequence
  Synthetically generated
gggttacgaaccgggactacaaagggtttctccatcagtgcacactctaaagaaaatctagcaccaacccaggttagccgctatacatgaccggacgtcaccaaccctatggaaggatatgatgctgttaggtacatggattagttgctgtctagattacgtgcaggtaattaacacatccaggagaaaacactggacagtgcgtacgtacttaattagtgatcaaccaaaaatatgcatgatattgcaatccagctaattacgttaggtgcacataagccagatgtagtataagctaagccagccgtttccatacgacatatgcataaggatgcaattatcctgatgcacgcttgatttaatttgatgggatgcgtacatattttgattccttgtcctaaagtatgcaaaaatccctgtccatcaggtgtgttgtctacacacggctatgtctcattgtgttatatatgttgacttgaactttttcgcaaaatggatttcattaattggttccttttcaaagtgactttagtatattataggaaacggtgaagatgacctctataccacctaatttaatcgaccttgtgttgttaggtggcacatcaaatatcattatctatatctctacctataccttatataagtaacccaggggaaaaaaatcgaacccatgaattgtgagatcacaattcagagattaaaacaaggtatgccaaatatgagtatatagtataccatataaaataactcaaattcgaattaagaataaacatgaaaaatagcaattggctttgaagattaattacgtactctgctgaaaaaaaaaccaaaagaatctggaaagaacataagtgtgaaatttcagtatcttctcaacagtacagaagaattatttatattaaaaattgcatcatttttttggaaaagggatatatatatatacacacacacacaaacacacacacacacacacacacacacattcagacagaacataaccatatagccatgcacccgaccgatgctaacggctcacactcgccaaagtatggctagctaaattttgatcccatgaattttctatactctagcaggcctatcttcagccaacatctttttaatttcttccctaaccagaaattggtcatctaaggagtcaatttttattttctctaagttcaaacaaacttattttttttggggcgaatgtacatctaacaggacccacaggtagacgtgattttttctaaaaaaagatgttataaaattgcaccttgtatcaaaatactttgacatatatacattccaaagggagaatatgttgctagacacttgtaataattgattggttcagaaattaatcactaattgtccgtaaagggtttaattaatcgttagtggttacagttggatgatatatgccaaaatgaacggtgaatttcgaatctttcttgcatctggtggctattaattactttaggagtaaatttaaaaaactatatgtatgttaatatcaaactatcacaaactacttatttgagacattgtattataaactatagatttcgcaccaaaaatatcacaaaactacatatttaaagcccaaactcaaaaaactatggttttgttatataaacgttatatgtaaatatgtcaaccaaacgtcgtcacatggagaaaccagataaaacagactgacagtctggagaaccattaaaatcttacaagatcacacactgcaaactgcatgctctctctccctctcaacgcctatataagcacatccatcccccctatgatcaaagcatcacagaaaccataaacacacaggcatctgattagagaaatcta

SEQ ID NO: 25 - DNA, 1000 bp, Arabidopsis thaliana
  misc_feature (0)...(0): 5′-UTR YP0102a
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 26 - DNA, 1971 bp, Artificial Sequence
  Synthetically generated
tcagggaggtatgtggttatcttgccttcaagttttacattttgtttccatgatattcacatgctgtattgcaggttattgctctttgtgatcatccatgcttgttggaaaaggaggaaaccaaatcattgttcaggtgaatatcggcacctttatttcatcagcatcaaacagatatgcagagaacttaaatggagatatctagtgcaaacactcacattcctttagtttgcttaccatatacttcatccttttgtttctctctactgattgagttttgactagaaatattacatgttagttgagcataggagtttcaaaaaccaaaatcttattgagaaattttcaaggtggtttatccctagttaaaagggctaggactaaatcgattaactatgcaactggcatatcaccctaacttaatttctaaaagagttctgctcatgaacttccataaatagttgactatcatactgaaatttgaaattctagtgagtatctgatgccccatctttgctgcagtgctgatgccatttctcgagcaacaacaattcttgcctcgattcctggaagagcaactggagcatacagccacagccaggtgagcagctcaggctgatacatttactcactacaaagaaaaaaaaagaatcttaatttcaccgtactcatttttcctagggcatcaaagggctgcgtgatgcaattgctgctggaattgcatcacgtgacggataccctgcaaatgcagacgacattttccttactgacggagcaagccctggagtaggaactttaccttctttttaaatcttactggacattttttgaataaacaggaagcagttcgaatctcattatgatgctattctccccctctgttttaggttcacatgatgatgcagttactgataaggaacgagaaagatggcattctctgcccaattcctcaatatcctttgtactcagcctccattgctcttcatggtggagctcttgtatgttttgaattctcagcacattttcaatatggctgcattcatgctgcaccaaagcctaattgagagcattttgttttaggtcccgtattatcttaatgaatcaacaggctggggtttggagatctctgaccttaagaagcaactcgaagattctcggttgaaaggcattgatgttagggctttggtagttatcaatccaggaaatccaactgggcaggtttgcattcattgctttcttgtctaatttggagagcatcttggattgttgcaatttctgttcacaccatattctgcatgtatctacctaaggcatatatatttgcaattcttgtatctttttatgtgattttccattgttagggaacatatgtatttttgtttgtctgcaatgtgcatgaagcatttgcagctggtgcaggtacccaacaaaagaactgtaatcatgttttaattcatttgcaggttcttgctgaggaaaaccaacgggacatagtgaagttctgcaaaaatgagggacttgttcttctggctgatgaggtaagcgattgttacttgagcaactccacaacaaactttcagctgcttaattccttttcgctgtgctgtctgtaacatcaacactattcatattgataggtgtaccaagagaacatctatgttgacaacaagaaatttaactctttcaagaagatagcgagatccatgggatacaacgaggatgatctccctttagtatcatttcaatctgtttctaagggtaaatacgatgatctgttttcttattttctattggcactggattctcaaaaggattttcttgctgacaacaggatattatggtgaatgtggcaaaagaggaggctacatggagattactggcttcagtgctccagttagagagcagatctacaaagtggcgtcagtgaacttatgttccaatatcactggccagatccttgccagcctcgtc

SEQ ID NO: 27 - DNA, 1993 bp, Artificial Sequence
  Synthetically generated
gcttggaacagcagagatttggcataagaacaaatttgtaaatgtaatttgtatgatattgtagctagactgtttggagcaaatcaattccgtggcgctacaaaagaatctctttttgaaaaaactaaaattacaacaaaaacggcacgctttgcaaaccatggtgtaacgtttgcccacaacaacctgtataagaaaacaagctttacagcttcgtacaactctggttagcaaactaattttgtcacgctaaggaatcagtttctcatagcaccgaccagtttcacctataaattagaggatactgcacagcccttgatcacaatacagtgcatttctacaatcttttgttgcccattcatctgggttttcttctgcttctttttttttcctagagagtacggttttctttgtaattctttaatttgttgcaaccatgaatgtattggcatctaagatcttcccttcccgctccaatgttgccagcgagcaacaacaatcgaagcgcgagaaagcaactattgatgacgctaagaactcgtccaagaacaaaaatcttgaccgcagtgtcgatgaggtaaccgatcttccccacaaaacatattcataaataccattacttgattttttttatggaattccttattcatgtagaacatattttctatttgatgaattctccatgcatgatgtttcaatcttcttttttttattgtgtggagtatataaaagtaattagaatttgtagcacctggacatatgcagcaaattattcatctactactatagttcggatttatttttatcgatgcaaattggatttggatagaaatgtacattcttttattttagtcagaataaaagtttcttctatctagaatatactataataacatatctatctaaaacaaatatggtacaacacacttgcaactagcagcaagttccctgaaagatgtttgtctaatgctatggtgatctctttcactacagtttggtgtatgtgtgtccatagtagaatatgagtcctgcaaaagcaaacatcatcatgccaacaaaaatggcccatgtgccatcaataattcaaggtgcccgttgatgagtaacagaacatttgattgtgtcaccctaccacaaacacacatggaaggccattgcattccctataaggacatcatggtcattccaaaatgtactgacacctgctcaatgcagacaaaaaccccttcaaaaaacagaagaatctccctcttaaaaaaactgattaaatgattatttctgaaataaaaatgttgagtttttatttttaaatagtttatatcattctattcttttagaaacgtagtacaaacatagatacttacagcgtgcgcatactcatctatataaatgcacacctctgaaaaactaaagagaagtggaaaaaatggcaagatttactaataattagattatagtttttcacatctaataggaaaattatagattaaataattttttgaaagaaaaaaatatttgaaaacttatttattttcaagtatttgaaattatttaaataaagagtaaattttagaaaactacaactacagtgaaaaaactatcagtttgctataacttttacgtgatatgttgctacagttgtcacctacatgtcctgtagcagtatatcacatcaaagttgtagttttgtgataatttttcatgctattggtgcaaaaaactgaaatagatcattaatattacagcaaactgatagttctatcactgtagttatagttttctgaaatttaagatctaaaagaagaaaaaaagggggggggggggggtgagatttacacacagccacacgacacgaggcagggctaccccactagacaatctgtccactcaccactggcctcacttccttgatctcttctcgtcttctccaccccgcacgcggccacccccgcagggaccccgtgacccgcgcccgcgcccgcgcctcaccgca

SEQ ID NO: 28 - protein, 1534 aa, Arabidopsis thaliana
  PEPTIDE (0)...(0): gi10177145 DNA (cytosine-5)-methyltransferase (MET1) (At5g49160)
MVENGAKAAKRKKRPLPEIQEVEDVPRTRRPRRAAACTSFKEKSIRVCEKSATIEVKKQQIVEEEFLALRLTALETDVEDRPTRRLNDFVLFDSDGVPQPLEMLEIHDIFVSGAILPSDVCTDKEKEKGVRCTSFGRVEHWSISGYEDGSPVIWISTELADYDCRKPAASYRKVYDYFYEKARASVAVYKKLSKSSGGDPDIGLEELLAAVVRSMSSGSKYFSSGAAIIDFVISQGDFIYNQLAGLDETAKKHESSYVEIPVLVALREKSSKIDKPLQRERNPSNGVRIKEVSQVAESEALTSDQLVDGTDDDRRYAILLQDEENRKSMQQPRKNSSSGSASNMFYIKINEDEIANDYPLPSYYKTSEEETDELILYDASYEVQSEHLPHRMLHNWALYNSDLRFISLELLPMKQCDDIDVNIFGSGVVTDDNGSWISLNDPDSGSQSHDPDGMCIFLSQIKEWMIEFGSDDIISISIRTDVAWYRLGKPSKLYAPWWKPVLKTARVGISILTFLRVESRVARLSFADVTKRLSGLQANDKAYISSDPLAVERYLVVHGQIILQLFAVYPDDNVKRCPFVVGLASKLEDRHHTKWIIKKKKISLKELNLNPRAGMAPVASKRKAMQATTTRLVNRIWGEFYSNYSPEDPLQATAAENGEDEVEEEGGNGEEEVEEEGENGLTEDTVPEPVEVQKPHTPKKIRGSSGKREIKWDGESLGKTSAGEPLYQQALVGGEMVAVGGAVTLEVDDPDEMPAIYFVEYMFESTDHCKMLHGRFLQRGSMTVLGNAANERELFLTNECMTTQLKDIKGVASFEIRSRPWGHQYRKKNITADKLDWARALERKVKDLPTEYYCKSLYSPERGGFFSLPLSDIGRSSGFCTSCKIREDEEKRSTIKLNVSKTGFFINGIEYSVEDFVYVNPDSIGGLKEGSKTSFKSGRNIGLRAYVVCQLLEIVPKESRKADLGSFDVKVRRFYRPEDVSAEKAYASDIQELYFSQDTVVLPPGALEGKCEVRKKSDMPLSREYPISDHIFFCDLFFDTSKGSLKQLPANMKPKFSTIKDDTLLRKKKGKGVESEIESEIVKPVEPPKEIRLATLDIFAGCGGLSHGLKKAGVSDAKWAIEYEEPAGQAFKQNHPESTVFVDNCNVILRAIMEKGGDQDDCVSTTEANELAAKLTEEQKSTLPLPGQVDFINGGPPCQGFSGMNRFNQSSWSKVQCEMILAFLSFADYFRPRYFLLENVRTFVSFNKGQTFQLTLASLLEMGYQVRFGILEAGAYGVSQSRKRAFIWAAAPEEVLPEWPEPMHVFGVPKLKISLSQGLHYAAVRSTALGAPFRPITVRDTIGDLPSVENGDSRTNKEYKEVAVSWFQKEIRGNTIALTDHICKAMNELNLIRCKLIPTRPGADWHDLPKRKVTLSDGRVEEMIPFCLPNTAERHNGWKGLYGRLDWQGNFPTSVTDPQPMGKVGMCFHPEQHRILTVRECARSQGFPDSYEFAGNINHKHRQIGNAVPPPLAFALGRKLKEALHLKKSPQHQP

SEQ ID NO: 29 - DNA, 4845 bp, Arabidopsis thaliana
  misc_feature (0)...(0): NM_124293.3; GI42568413;  DNA (cytosine-5-)-methyltransferase (ATHIM), (At5g49160) mRNA, complete cds
gaccaattagggtttcgcaatcttccagtagatttcgcttctcaacggattttgaaaatggtggaaaatggggctaaagctgcgaagcgaaagaagagaccacttccagagattcaagaggtagaagatgtacctaggacgaggagaccaaggcgtgctgcagcgtgtaccagtttcaaggagaaatctattcgagtctgtgagaaatctgctactattgaagtaaagaaacagcagattgtggaggaagagtttctcgcgttacggttaacggctctggaaactgatgttgaagatcgtccaaccaggagactgaatgattttgttttgtttgattcagatggagttccacaacctctggagatgttggagattcatgacatattcgtttcaggtgctatcttaccttcagatgtgtgtactgataaggagaaagagaagggtgtgaggtgtacatcgtttggacgggttgagcattggagtatctctggttatgaagatggttcccctgttatttggatctcaacggaattggcggattatgattgtcgtaaacctgctgctagctacaggaaggtttatgattacttctatgagaaagctcgtgcttcagtggctgtgtataagaaattgtccaagtcatctggtggggatcctgatataggtcttgaggagttacttgcggcggttgtcagatcaatgagcagtggaagcaagtacttttctagtggtgcggcaatcatcgattttgttatatcccagggagattttatatataaccaactcgctggtttggatgagacagccaagaaacatgaatcaagctatgttgagattcctgttcttgtagctctcagagagaagagtagtaagattgacaagcctctgcagagggaaagaaacccatctaatggtgtgaggattaaagaagtttctcaagttgcggagagcgaggccttgacatctgatcaactggttgatggtactgatgatgacagaagatatgctatactcttacaagacgaagagaataggaaatctatgcaacagcccagaaaaaacagcagctcaggttctgcttcaaatatgttctacattaagataaatgaagatgagattgccaatgattatcctctcccatcgtactataagacctccgaagaagaaacagatgaacttatactttatgatgcttcctatgaggttcaatctgaacacctgcctcacaggatgcttcacaactgggctctttataactctgatttacgattcatatcactggaacttctaccgatgaaacaatgtgatgatattgatgtcaacatttttgggtcaggtgtggtgactgatgataatggaagttggatttctttaaacgatcctgacagcggttctcagtcacacgatcctgatgggatgtgcatattcctcagtcaaattaaagaatggatgattgagtttgggagcgatgatattatctccatttctatacgaacagatgtggcctggtaccgtcttgggaaaccatcaaaactttatgccccttggtggaaacctgttctgaaaacagcaagggttgggataagcattcttacttttcttagggtggaaagtagggttgctaggctttcatttgcagatgtcacaaaaagactgtctgggttacaggcgaatgataaagcttacatttcttctgaccccttggctgttgagagatatttggtcgtccatgggcaaattattttacagctttttgcagtttatccggacgacaatgtcaaaaggtgtccatttgttgttggtcttgcaagcaaattggaggataggcaccacacaaaatggatcatcaagaagaagaaaatttcgctgaaggaactgaatctgaatccaagggcaggcatggcaccagtagcatcgaagaggaaagctatgcaagcaacaacaactcgcctggtcaacagaatttggggagagttttactccaattactctccagaggatccattgcaggcgactgctgcagaaaatggggaggatgaggtggaagaggaaggcggaaatggggaggaagaggttgaagaggaaggtgaaaatggtctcacagaggacactgtaccagaacctgttgaggttcagaagcctcatactcctaagaaaatccgaggcagttctggaaaaagggaaataaaatgggatggtgagagtctaggaaaaacttctgctggcgagcctctctatcaacaagcccttgttggaggggaaatggtggctgtaggtggcgctgtcaccttggaagttgatgatccagatgaaatgccggccatctattttgtggagtacatgttcgaaagtacagatcactgcaaaatgttacatggtagattcttacaaagaggatctatgactgttctggggaatgctgctaacgagagggaactattcctgactaatgaatgcatgactacacagctcaaggacattaaaggagtagccagttttgagattcgatcaaggccatgggggcatcagtataggaaaaagaacatcactgcggataagcttgactgggctagagcattagaaagaaaagtaaaagatttgccaacagagtattactgcaaaagcttgtactcacctgagagagggggattctttagtcttccactaagtgatattggtcgcagttctgggttctgcacttcatgtaagataagggaggatgaagagaagaggtctacaattaaactaaatgtttcaaagacaggctttttcatcaatgggattgagtattctgttgaggattttgtctatgtcaaccctgactctattggtgggttgaaggagggtagtaaaacttcttttaagtctgggcgaaacattgggttaagagcgtatgttgtttgccaattgctggaaattgttccaaaggaatctagaaaggctgatttgggttcctttgatgttaaagtgagaaggttttataggcctgaggatgtttctgcagagaaggcctatgcttcagacatccaagaattgtatttcagccaggacacagttgttctccctccaggtgctctagagggaaaatgtgaagtaagaaagaaaagtgatatgcccttatcccgtgaatatccaatatcagaccatattttcttctgtgatcttttctttgacacctccaaaggttctctcaagcagctgcccgccaatatgaagccaaagttctctactattaaggacgacacacttttaagaaagaaaaagggaaagggagtagagagtgaaattgagtctgagattgtcaagcctgttgagccacctaaagagattcgtctggctactctagatatttttgctggttgtggtggcctgtctcatggactgaaaaaggcgggtgtatctgatgcaaagtgggcgattgagtatgaagagccagctgggcaggcttttaaacaaaaccatcctgagtcaacagtttttgttgacaactgcaatgtgattcttagggctataatggagaaaggtggagatcaagatgattgtgtctctactacagaggcaaatgaattagcagctaaactaactgaggagcagaagagtactctgccactgcctggtcaagtggacttcatcaatggtggacctccatgtcagggattttctggtatgaacaggttcaaccaaagctcttggagtaaagttcagtgtgaaatgatattagcattcttgtcctttgctgactatttccggccaaggtattttcttctggagaacgtgaggacctttgtgtcattcaataaagggcagacatttcagcttactttggcttcccttctcgaaatgggttaccaggtgagatttggaatcctggaggccggtgcatatggagtatcccaatctcgtaaacgagctttcatttgggctgctgcaccagaagaagttctccctgaatggcctgagccgatgcatgtctttggtgttccaaagttgaaaatctcactatctcaaggtttacattatgctgctgttcgtagtactgcacttggtgcccctttccgtccaatcaccgtgagagacacaattggtgatcttccatcagtagaaaacggagactctaggacaaacaaagagtataaagaggttgcagtctcgtggttccaaaaggagataagaggaaacacgattgctctcactgatcatatctgcaaggctatgaatgagcttaacctcattcgatgcaaattaatcccaactaggcctggggctgattggcatgacttgccaaagagaaaggttacgttatctgatgggcgcgtagaagaaatgattcctttttgtctcccaaacacagctgagcgccacaacggttggaagggactatatgggagattagattggcaaggaaactttccgacttccgtcacggatcctcagcccatgggtaaggttggaatgtgctttcatcctgaacagcacagaatccttacagtccgtgaatgcgcccgatctcaggggtttccggatagctacgagtttgcagggaacataaatcacaagcacaggcagattgggaatgcagtccctccaccattggcatttgctctaggtcgtaagctcaaagaagccctacatctcaagaagtctcctcaacaccaaccctagataaccacccaaatttggcatttcctttttcaataatattagtcattatgatccttgtcttgaatgaaactcattggtgctgatacttttgataaagaaagcctacgaagagtttttgtatattccgtattcggattgaaaaatctcattatacaagcaagcaatgatgtctatagactatga

SEQ ID NO: 30 - protein, 1564 aa, Prunus persica
  PEPTIDE (0)...(0): gi | 37039880 | gb | AAM96952.1 |  DNA cytosine-5-methyltransferase
MGSAAAAEAAEAAALLEAKGANGTKPPSSSSSGMTKKKKGKQDSQKAAPKAKKRNLPQSSEEEPSRSRKMPKRAAACKDFKDRSVHISEKSSLIESKEDQIVEEEILAVRLTCGPDQDAVRPNRRLTDFVLHDATGSAQPLEMLEVSDMFISGAILPLNESSDKDKGRSVRCEGFGRIESWDISGYEDGSPVIWLSTEVADYDCRKPASSYKKYFDQFFEKARACIEVYKKLSKSNSDNSDPTLDELLAGIARSMSGSKFFSGSASVKDFVLSQGEFIYAQVIGLEETSKKNDRPFAELPVLAALRDESIKRGNFVQSKPGISSGTLKIGGENGVDSAGSSVVEAEENEDAKLAKLLQEEEYWKSMKQRKRQGPASVSSKYYIKINEDEIANDYPLPAYYKNCIEETDEFIVFDNEFDICNADDLPRSMLHNWCLYNSDSRLISLELLPMKPCADIDVTIFGSGVMSEDDGSGFCLDSDGTSSGPGAQDADGMPIYLSAIKEWMIELGASMVSISIRTDMAWYRLGKPSKQYALWYEPILRTAKIGRSIITMLKDQSRVARLSFADVIKRLSGFQKDHCAYISSDPAFVEKYVVVHGQIILQLFSEFPDAQIKKCPFVIGLTKKMEERHHTKWLVKKKKLVEKSESNLNPRASMAPVVSKRKTMQATTTRLINRIWGEYYSNYSPEDSKEGDIGEKKEEEEVEEEDVEEDDVEENPTVMEQAQKPSSISRQTKSCLNNREILWEGEPVGQTCSGEALYKRAILWGEEISVGGAVLVELDESHELPAIYFVEYMYETLNGSKMFHGRVMERGSQTVLGNTANEREVFLTNECTNLALKEVKQAAAVGIKVMPWGHQYRKDNADANRTDRARAEERKRKGLPTEYYCKSLYCPERGAFLSLSRDTMGLGSGACHSCKMNEAEEAKEVFKVNSSKTGFVYRGVEYSVHDYVYVSPHYFGVERMETEIFKAGRNLVLKAYVVCQVLEIVVMKESKRPEIESTQVKVRRFFRPEDISVEKAYSSDIREVYYSEQTHIVPVDNIERKCEVRKKSDLPVCNAPVIFQHIFFCEHLYDPSKGSIKQLPAHIKLRYSTGGGHADSRKRKGKCKEGENVSEVENQRVDSEQKRLATLDIFAGCGGLSNGLRQSGASITKWAIEYEEPAGDAFKLNHPESLVFINNCNVILRAVMEKCGDTDDCIATSEAAELAASLDEEVKNDLPLPGQVDFINGGPPCRGFSGMNRFTQSPWIKFHCKMIWACLAFADYFRPKLFPLENVRKFVSFNKGQTFQLTLASLLEMGYQVRFGILEAGAYGISQSRKRAFIWAAAPEEVLPEWPEPMHVFGVPKLKISLSQGLHYAAVRSTALGAPFRPITVRDTIGDLPSVENGDSRTNKEYKEVAVSWFQKEIRGNTIALTDHICKAMNELNLIRCKLIPTRPGADWHDLPKRKVTLSDGRVEEMTPFCLPNTAERHNGWKGLYGRLDWQGNFPTSVTDPQPMGKVGMCFHLEQHRILTVRECARSQGFPDSYEFAGNINHKHRQIGNAVPPTLAYALGTKLKEAIDSKRLSSQE

SEQ ID NO: 31 - DNA, 4992 bp, Prunus persica
  misc_feature (0)...(0): AY128652.1; GI37039879;  DNA cytosine-5-methyltransferase mRNA, complete cds
tcagccctctcattacaccccacattgcgcattctagggtttcactggcgagtggggagaaatgggttccgcagcggcagcagaagcggcagaagcagcagcgctcttggaggccaaaggtgccaatgggactaaaccaccatcttcgtcatcttcaggaatgacgaagaagaagaagggtaaacaagattcccaaaaggcagcacctaaagctaagaagcgaaatttgcctcagagcagtgaagaagagccttcccgatctcggaaaatgccgaagcgggctgctgcttgcaaagactttaaggataggtctgttcatatttctgagaagtctagccttattgaaagcaaggaggaccagatagtggaggaagaaattcttgccgtacgcctgacttgtggcccggaccaagatgctgtgcgcccaaacagaagactgactgattttgttttgcatgatgcaactggttccgcacaaccccttgagatgttggaagtttctgacatgtttatatctggtgctatattgcctctcaatgaaagttctgacaaggacaagggaagaagtgttagatgtgaaggtttcgggcggatagaatcttgggacatctctggttatgaagatggctcccccgtaatatggctttcaactgaagttgctgattatgattgccgtaaaccggccagtagctacaagaaatactttgatcaattctttgagaaagcgcgtgcttgcatagaggtttacaagaagctgtctaaatccaactccgacaactccgaccccactcttgatgaattgcttgctggtattgcacgatcaatgagcgggagcaaattcttttctgggagtgcatctgtcaaagactttgttctatctcaaggcgagtttatttatgctcaagtaataggtctggaggaaacatcaaagaagaacgatcggccatttgcagagttacctgtccttgctgccctcagagatgagagtataaagcgtggaaattttgtgcaatcaaaaccgggaatttcaagtggtactttaaagattggtggagagaacggagtggattcagctggttcatccgtagttgaagctgaggaaaatgaggatgcaaagttggcaaaactcttgcaagaggaagaatactggaagtcaatgaaacaaagaaagcgccagggtcctgcctctgtgtcaagcaaatactacatcaaaattaatgaagatgaaattgccaatgattatcctctacccgcttattacaagaattgcattgaagaaactgatgagttcatagtttttgacaatgagtttgatatctgtaatgctgatgaccttcctcgaagtatgcttcataattggtgtctatacaactcggactcaagattgatttcgctcgagcttcttccaatgaaaccctgcgcagacattgatgttaccattttcgggtcaggggttatgagtgaagatgatggaagtggcttttgtcttgattctgatggtacttcaagtggtccaggagcccaggatgctgatggaatgccaatttacttgagtgcgataaaggaatggatgattgaattgggagcatcaatggtttcaatatcaatccgaacagatatggcctggtacagacttggcaagccatctaagcagtatgctctgtggtatgaaccaattctgagaacagcaaagattgggagaagtataatcactatgctgaaagatcaaagtcgagtagcacggctttctttcgcagatgtcattaagagactgtcagggttccaaaaggaccattgtgcttacatttcttctgatccagcatttgttgagaagtatgtcgttgtccatggacagataatactgcaactgttttcagaatttccagatgcgcagattaaaaaatgtccatttgtgattggtcttacaaagaaaatggaggagaggcaccatactaaatggttagtaaagaagaagaagcttgtggaaaagagtgaatcaaatttgaacccaagggcatcaatggcacctgtggtttccaagaggaagacaatgcaagctacaacaacaaggctgatcaacagaatctggggggagtactattcaaactactctccagaagattcgaaggagggagatattggagaaaagaaagaggaggaggaagttgaagaagaggatgtagaagaggatgatgtagaagagaatccaactgtaatggagcaagcccagaagccttcttcaatttcaagacaaaccaaatcatgcctcaacaacagggaaattttgtgggaaggggagccagtgggccaaacatgttctggtgaagctctttataagcgtgccattctttggggagaagaaatttctgttggcggtgctgttttggtggaacttgatgaatcccatgaacttcctgccatttattttgtggagtatatgtatgaaacattgaatggaagcaaaatgtttcatggaagagtgatggagcgaggatcccagactgttcttggcaacactgccaatgagagggaggtatttttgacaaatgagtgcacaaatttggcattaaaggaagttaaacaggcagctgctgtgggcattaaagtaatgccgtgggggcatcagtataggaaggataatgctgatgctaacagaactgatagagcaagggcagaagagaggaagaggaagggtttgccgactgaatattactgtaaaagcttgtattgcccagagagaggtgctttccttagtctttcacgtgatactatgggtctgggttctggtgcctgccactcttgcaaaatgaatgaagccgaggaggccaaggaagtatttaaagtgaattcatcaaaaactggttttgtatacaggggagttgagtactcagttcatgattatgtctatgtaagtccccattattttggtgtggaaaggatggaaactgaaattttcaaggctggaaggaatttggtgctgaaagcttatgtcgtgtgccaagtgctggagatagttgttatgaaggagtctaaacgacctgaaatagaatctacccaggttaaagtaagaagatttttcagaccagaggacatatctgttgagaaggcatacagttcggatattagagaggtctactacagtgaacaaacacacatcgtgcctgttgataatatagaaagaaaatgtgaagtcagaaagaagagtgatcttccagtatgtaatgctcctgtcattttccagcatattttcttctgtgaacatctatatgatccttctaaagggtctattaagcagttgccagctcacatcaaactgaggtactcaacaggaggtgggcatgctgattctagaaagagaaagggcaagtgcaaagaaggagaaaatgtttcagaagttgagaaccagagagttgattctgagcagaaacgcctagccacattggatatatttgctggttgcggtggcttgtctaatgggttgcgtcagtctggtgcttcaataaccaagtgggcaattgagtatgaagagcctgctggggatgctttcaaactcaaccatcctgagtcattggtttttatcaataactgcaatgtgatcttaagggccgtaatggaaaaatgtggggacacagatgattgtattgcaacttctgaagctgctgaattggctgcatcacttgatgaggaggttaaaaatgatttgccgttgccggggcaggtagatttcatcaatggaggacctccatgccggggtttctctggaatgaataggttcacccaaagcccttggattaaatttcattgtaaaatgatttgggcttgcttagcctttgccgactacttccggccaaagttgttcccgctggagaatgtgaggaaatttgtgtcattcaataaagggcagacatttcagcttactttggcttcccttctcgaaatgggttaccaggtgagatttggaatcctggaggccggtgcatatggaatatcccaatctcgtaaacgagctttcatttgggctgctgcaccagaagaagttctccctgaatggcctgagccgatgcatgtctttggtgttccaaagttgaaaatctcactatctcaaggtttacattatgctgctgttcgtagtactgcacttggtgcccctttccgtccaatcaccgtgagagacacaattggtgatcttccatcagtagaaaacggagactctaggacaaacaaagagtataaagaggttgcagtctcgtggttccaaaaggagataagaggaaacacgattgctctcactgatcatatctgcaaggctatgaatgagcttaacctcattcgatgcaaattaatcccaactaggcctggggctgattggcatgacttgccaaagagaaaggttacgttatctgatgggcgcgtagaagaaatgactcctttttgtctcccaaacacagctgagcgccacaacggttggaagggactatatgggagattagattggcaaggaaactttccgacttccgtcacggatcctcagcccatgggtaaggttggaatgtgctttcatcttgaacagcacagaatccttacagtccgtgaatgcgcccgttctcaggggtttccggatagctacgagtttgcagggaacataaatcacaagcacaggcagattgggaatgcagttcctcctactttggcctatgcattggggactaaactcaaggaagcaattgacagcaagaggttgtcttcacaagagtaagagtggttgttgttgtttgtttctatgtaatactgatagttccatttggttgccttctaaggcaaaaacacagctcagtttgttgtctttgattttcttcttatattgtgtttgtaaacttgtcttgattgaggaacttcaattaaatacacacaagcatttttcttcaggagacaagtgtcacaaaagtttggtacatatatatatttgaaattattttactttatttagaaaaa

SEQ ID NO: 32 - protein, 265 aa, Glycine max
  PEPTIDE (0)...(0): Ceres Clone520982 Met1 homolog
MEKCGDTDDCISTSEAAELAAKLDEKEISSLPMPGQVDFINGGPPCQGFSGMNRFNQSSWSKVQCEMILAFLSFADYFRPRYFLLENVRNFVSFNKGQTFRLTLASLLEMGYQVRFGILEAGAYGVSQSRKRAFIWAASPEDVLPEWPEPMHVFSAPELKITLSENVQYAAVRSTANGAPLRSITVQDTIGDLPAVGNGASKGNMEYQNDPVSWFQKKIRGDMVVLTDHISKEMNELNLIRCQKIPKRPGADWRDLPEEKVKLNI

SEQ ID NO: 33 - DNA, 1594 bp, Glycine max
  misc_feature (0)...(0): Ceres Clone520982 Met1 homolog
aattgcaatgttattcttagggctgtaatggagaagtgtggggacacagatgattgtatctcaacatccgaagctgcagaattggctgcaaagcttgatgagaaggaaataagtagtttaccaatgcctggacaagttgatttcatcaatggtggtcctccatgtcagggtttctctgggatgaataggtttaaccagagcagttggagtaaagtccagtgtgagatgatattggcattcttatcctttgccgattatttccggccaaggtatttcttgttggagaatgtgaggaactttgtgtctttcaataaagggcagacattccgtttaactttggcttcacttcttgagatgggctatcaggtgaggtttggtatccttgaggctggagcatatggggtttcccagtcaagaaaaagggcattcatatgggcagcctctcctgaggatgtgcttcctgaatggcctgaaccaatgcatgtcttttcggcccctgagttgaagattacattatcagaaaatgtccagtatgctgctgtccgcagtactgcaaatggtgctccattacgttcaataactgttcaagatactattggtgatctcccagctgttggcaatggagcctcaaaaggaaacatggagtatcaaaatgatccagtctcatggtttcaaaagaagattcgaggtgatatggttgtcttgactgatcatatatcaaaggagatgaatgaattgaacttgattcgatgccagaaaattcccaagagaccaggcgctgattggcgtgaccttccagaagaaaaggtgaagttaaatatttgagttttagcataacattttttgtgatctatctaatatgtgaaatctaatgaaatgcagataaaattgtctactggacaagttgttgatttgataccatggtgcttgccaaacacggctaagcggcacaatcagtggaagggactgtttggcaggttggattggcaagggaatttcccaacttccattactgaccctcagccaatggggaaggttggaatgtgcttccaccctgaccaagataggattcttactgttcgtgaatgtgctcggtctcaaggcttcccagatagctatcaatttgctggcaatatcatacacaagcaccggcagattggtaatgctgtgcctcctcctctggcatctgcattggggagaaagctcaaggaagcagtggacagtaagagctccacttagaagatggggcttctacattttttgaaatatcatgcttattgtattcatatcagtcaccaagatattgcaaatcattattcagggttccagaaactagaaacccttgtatatagtgatatccattggtcatttgttttgaggctaattccttgtttaactttcctcaaccaaggaattgtatggatgatgttatgatgttcattttctatcaactagtattttcttgattagataatattttggctgtttatgacagaaatggctgggaatttagaattacctcccaatgtatatagttgacaattgagaccaattttgtcattttttttaacttgttatgaatatttgttgttgc

SEQ ID NO: 34 - protein, 1554 aa, Pisum sativum
  PEPTIDE (0)...(0): gi | 2654108 | gb | AAC49931.1 | cytosine-5 DNA methyltransferase
MGSASLLNPSDSSLPGGKDSTSKEEPVSNTEGEVMAGGKQKKRSLSESSEQPAPTRKVPKRSASAASKNLKEKSFSISDKSCLVETKKDQVAEGELLAVRMTAGQEDDRPNRRLTDFILHDESGAAQALEMLEIKDLFITGLILPLEGNADKKKEQGVRCHGFGRIESWDISGYEDGSPVIWISTEIADYDCQKPAGTYKKYYDLFFEKARACLEVYKKLAKSSGGDPDISLDELLAGMARSMSGSKYFSGTASLKEFIISQGDFIYKQLIGLDTMLKANDKGFEDIPALIALRDESKKQAHFANTQVRPSNATLRIGSGIVDEEKKNQMDSVDEEDEDAKLARLLQDEEYWKSNRQRKNSRSSSSSNKFYIKINEDEIANDYPLPAYYKTSLQETDEFIVFDNDCDIYDTEDPSRSMLHNWALYNSDSRLISLELLPMKPCSEMDVTIFGSGTMTSDDGSGFNLDTEAGQSSVASGAQDTDGIPIYLSAIKEWMIEFGSSMVFISIRTDLAGIGLGKPSKQYTPWYDTVLKTARIAISIITLLKEQSRVSRLSFPDVIKKVSEYTQDNKSYISSDPLAVERYIVVHGQIILQLFAEFPDDKIRKSPFVTGLMNKMEERHHTKWLVKKKKLSPKSEPNLNPRAAMAPVVSKRKAMQATATKLINRIWGEYYSNHLPEESKEGTAIEEKDDDEAEEQEENEDEDAEEETVLLEETLKPRIVSKQIKAFSDDGEVRWEGVPERKTSSGLPLYKQAIIHGGSCFCGNICVSRKLMNQMSFLIYITLNICLNPKNGEKMFHGRMMQHGCHTVLGNAASEREVFLTNECRDLGLQDVKQINVASIRKTPWGHQHRKASNAAGKIDRERADERKKKGLPTEYYCKSLYWPERGAFFSLPFDTLGLGSGVCHSCNIQEADKAKEIFKVNSSKSSFVLDGTEYSLNDYVYVSPFEFEEKIEQGTHKSGRNVGLKAFVVCQVLEIIAKKETKQAEIKSTELKVRRFFRPEDVSSEKAYCSDVQEVYFSDETYTISVQSVEGKCEVRKKIDIPEGSAPGAFHNVFFCELLYDPATGSLKKLPSHIKVKYSSGPTADNAARKKKGKCKEGDSISVPDIKSKTSNENRLATLDIFAGCGALSEGLHKSGASSTKWAIEYEEPAGNAFKANHPEALVFINNCNVILRAIMEKCGDIDECISTAEAAELASKLDDKDLNSLPLPGQVDFINGGPPCQGFSGMNRFNTSTWSKVQCEMILAFLSFADYFRPRYFLLENVRNFVSFNKGQTFRLTLASLLEMGYQVRFGILEAGAFGVSQSRKRAFIWAASPEDVLPEWPEPMHVFSAPELKITLAENVQYAAVCSTANGAPLRAITVRDTIGELPAVGNGASRTNMEYQSDPISWFQKKIRGNMAVLTDHISKEMNELNLIRCQKIPKRPGCDWRDLPDEKIKLSTGQLVDLIPWCLPHTAKRHNQWKGLFGRLDWQGNFPTSITDPQPMGKVGMCFHPDQDRILTVRECARSQGFPDHYQFSGNIIHKHRQIGNAVPPPLAFALGRKLKEALDSKSAN

SEQ ID NO: 35 - DNA, 4910 bp, Pisum sativum
  misc_feature (0)...(0): AF034419.1; GI2654107; cytosine-5 DNA methyltransferase mRNA, complete cds
cttcagatctacaacccgcgttttggatacaaggaaaattttccaactcatgggttccgcttcgcttttgaatccctccgattcgtctctaccgggtggcaaggacagcacgagtaaagaagagcctgtttcaaacactgaaggggaagttatggctggtggtaagcaaaagaagcgaagtttgtcagagagcagtgagcagcctgctcctactcggaaagtgccgaaacgatctgcaagtgcagcaagtaaaaatttgaaggagaagtctttttccatatctgataagtcttgtcttgttgaaactaagaaggatcaggttgcagaaggagaattgctagcagtccgtatgactgctggacaagaggatgaccgcccaaatagaagacttacagactttatccttcatgatgaaagtggtgcagcacaggcacttgagatgcttgaaatcaaggatttattcatcactggacttatattgccactagaaggaaatgctgacaagaaaaaagagcaaggtgttagatgtcatggttttggtcgaattgagtcatgggacatatctggttatgaggatggctctccagtgatatggatttctactgagattgctgactatgattgccagaaaccagctggtacctacaaaaaatactatgatcttttctttgaaaaagctcgggcttgcttagaagtgtacaaaaaactagcaaagtcttctgggggagatcctgacataagccttgatgagttacttgctggcatggcacggtcaatgagtggtagcaagtacttttctggaactgcatcactaaaggaattcattatttctcagggtgattttatttataagcaactcattggtttagacacaatgttgaaggcaaatgacaaggggtttgaagatattcctgctttgattgctcttagagatgagagcaagaaacaagcacactttgcaaacacacaagtgaggccatcaaatgcgactttaaggattggttcgggaattgtagatgaagagaaaaagaatcagatggattctgtagatgaagaggatgaggatgcaaagttagctcgactattgcaggatgaagagtattggaaatctaacaggcagaggaaaaactctagatcatcatcttcatctaataaattctatatcaagattaatgaagatgagattgcaaatgattatcctctccctgcttattataaaacttctcttcaagaaacggatgaatttatagtttttgataatgactgtgacatatatgacactgaagatccttctagaagcatgttgcacaattgggctttatacaactctgattctagattgatttccctggaacttcttcccatgaaaccttgttcagagatggatgttacaatctttggatcaggtacaatgacttcagatgatggaagtggtttcaatcttgatacagaggctggccaatcttccgttgcttctggagcacaagacactgatggtattccaatttatctgagtgcaataaaagagtggatgattgaatttggatcatctatggttttcatatccatccgaacagatttggctggtataggacttggcaaaccatcaaagcagtacactccttggtatgacacagtattgaaaactgcaagaattgctataagcattatcacgttgttgaaggagcagagccgtgtatcacggctttcatttccagatgttataaaaaaagtatctgagtatactcaggacaataagtcatatatttcttctgatccattggctgtagaaagatatattgttgtccatggacagataattctgcaactatttgcagaatttccagatgacaagatcaggaagtctcctttcgtgactggtcttatgaacaaaatggaagaaaggcaccataccaaatggttagtgaagaagaagaaactgtcgccaaagagtgagccaaatttgaatcctagggcagcaatggctcctgttgtatctaaaaggaaagctatgcaagctacagcaacaaagctaatcaatagaatatggggtgagtattactcaaaccacttacccgaggaatcaaaagaaggaactgctattgaagaaaaggatgatgatgaagcagaggaacaggaagagaatgaagacgaggatgctgaggaagagacagtactgttggaggaaacactaaagccacgtatagtttccaaacagattaaagcattttctgatgatggagaggttagatgggaaggggttcccgaaaggaaaaccagttctggattgcctctttataagcaggcaattattcatggaggaagttgtttctgtgggaatatctgtgtcagtcggaagttgatgaatcagatgagcttcctgatatatattacattgaatatatgtttgaatccaaagaatggggaaaagatgtttcatggtaggatgatgcaacatggttgtcacactgttcttggcaatgccgcaagtgagagagaggtgtttttgactaatgagtgcagggatttgggactgcaagatgttaagcagataaatgttgcaagcatccgaaaaacaccttgggggcatcagcatcgaaaggctagtaatgctgcaggtaaaatcgatagagagagagctgatgaaaggaagaagaaaggactgcctactgaatattactgtaaaagcttgtactggcctgagaggggtgctttcttcagtcttccgtttgatacgctgggtttagggtctggtgtctgtcactcttgcaatatacaagaagctgacaaggcgaaggaaattttcaaagtaaattcgtctaagtctagttttgtattggatggaacagaatattctctcaatgactatgtttatgtaagcccttttgaatttgaggaaaagatagagcagggaactcataagagtgggaggaatgtagggctgaaagcttttgttgtatgccaagtgctcgagatcattgccaaaaaggaaacaaaacaagctgaaataaaatctacagaactcaaagtcagaagattctttcgaccagaagatgtatcaagtgagaaagcatactgctctgatgtacaagaggtgtatttcagtgatgaaacatatactatctctgttcaatctgtagaaggtaaatgtgaagtcaggaaaaagattgatatccctgaaggaagtgcccctggagcctttcacaatgtctttttctgtgaactcctgtatgatcctgccacaggatcgctcaagaagttgccatctcatatcaaagtaaaatattctagtggacctacagctgataatgcagctagaaagaaaaagggaaaatgtaaagagggagatagcatttcagtgcctgatataaaaagtaaaacatcaaatgaaaaccgtttagcaaccctggacatttttgcaggatgcggtgccttatcagaggggttgcataagtctggtgcttcatcaactaaatgggctattgaatatgaagaaccagctggcaatgcattcaaagctaatcatcctgaagctttggtgtttattaacaactgtaatgtaattctcagggctataatggagaaatgtggagatatagatgaatgtatctcaacagccgaggctgcagaattggcctctaagcttgatgataaggatttgaatagtttaccattacctgggcaagttgatttcattaatggggggcctccatgccagggtttctctgggatgaatagatttaacacaagcacttggagtaaagtccagtgtgagatgatattagcgttcttatcctttgctgattatttccggccgaggtatttcctcttggagaatgtgaggaactttgtgtcttttaataaaggacagactttccgtttaactttggcttcacttctcgagatgggttaccaggtgaggtttggtatcctcgaggctggagcttttggtgtttctcagtcaagaaaaagggcatttatatgggctgcctctccagaagatgtgcttcctgagtggccagaaccaatgcatgtcttctctgcccctgagttgaaaatcacattggcagaaaatgtccagtatgctgccgtctgcagtactgcaaatggtgctccgttacgggcaataactgttcgtgataccattggtgaactcccagctgttggcaatggagcctctaggacaaacatggagtatcaaagcgatcctatctcgtggtttcaaaagaagatccgaggcaatatggctgtcttgactgatcatatatcaaaggaaatgaatgagttgaacttgatccgatgtcagaaaattcctaagagaccaggttgtgattggcgtgatcttccagacgaaaagataaaactttcaactggacaacttgttgatttgataccatggtgcttgccacacacagctaagaggcataatcaatggaagggactgtttggtaggttagattggcaagggaatttcccaacttccatcaccgaccctcaaccaatggggaaggttggaatgtgcttccatcccgatcaagatagaattcttactgttcgtgaatgcgcccgatctcaaggctttccagaccactatcaattttctggtaacatcatacacaagcacaggcagattggtaacgcggttcctcctcctctggcatttgcattaggaaggaaactcaaggaagcattggatagtaagagcgccaattagaggattagggcgcatctttcaaaaagcatctttttatcatatagttttgtctttcagtgttctggaaacaacccaacccttgtatatagttgttttcttggctatttttcttagtttaatcaattctttgtttaaaaggattgatggaatggattatgctataaaactcattttttctatcaaaaaaaaaaaaaaa

SEQ ID NO: 36 - protein, 1545 aa, Daucus carota
  PEPTIDE (0)...(0): gi | 2895087 | gb | AAC39355.1 |  Met1-type cytosine DNA-methyltransferase
MGSSAVVDAPALDAGLETKKNKRKNADCDSEKTAVSGQKKQRAHALKSSETPVGSRKMPKRAAACADFKEKSIQISKKSSIIETKKDRSVDEEEVAVRLTAGQEDGRPCRRLTDFIFHNSDGIPQAFEMLEVDDLYISGLILPLEDSSQKEACSIKCEGFGRIENWALSGYEEGVPTIWVSTDVADYDCVKPSASYKKHYEHLFAKATACVEVYKKLSKSSGGNPDLSLDELLAGVVRGLSGMKCFSRSVSIKDFIISQGDFIYNQLVGLDETSKKTDQQFLELPVLIALREESSKHGDPSIGKVASTNGTLTIGPKIKDGENKKDSATEEDEGVKVARLLQEEEFWNSMKQKKGRGSSTSSNKYYIKINEDEIANDYPLPAYYKTANQETDEYIIFDGGADACYTDDLPRSMLHNWALYNSDSRLISLELLPMKGCADIDVTIFGSGVMTEDDGTGFNLDGDTSQSSSAGLGTANVDGIPIYLSAIKEWMIEFGSSMVFISIRTDMAWYRLGKPSKQYASWYEPVLKTARVAISIITLLKEQARVSRLSFMDVIKRVSEFEKGHPAYISSVPAAVERYVVVHGQIILQQFLEFPDEKIKKSAFVIGLTNKMEERHHTKWLMKKKKLLQRDEPNLNPRAALAPVVSKRKAMQATTTRLINRIWGEFYSNYSPEDMKEGITGEDKEEEEPEEQEEIEEEEEKETLTALEKTPTPTSTPRKTKSIPKVKDIRWNRKSVGETLSGEALYKQAIVYGTEIAVGGAVLVDDESAQLPAIYYVEYMFETLNGIKMLHGRMLQQGSLTILGNTANECEVFLTNDCMDFELADVKKAVVEIRSRPWGHQYRKVNANADKIYRAGVEERKKNGLETEYYCKSLYCPDKGAFLSLPLNSMGLGSGICSSCKLDKDLTEKEKFVVHSDKTSFVFNGTEYSIHDFLYVSPQQFSTERVGNETFKGGRNVGLKAYAICQLLEIIVPKAPKQAEPHSTEIKVRRFYRPEDISDEKAYCSDIREVYYSEETHTIDAETVEGRCEVRKKNDLPSCDAPTIFDHVFFCEYLYDPAKGSLKQLPPNIKLRYSAVKGAHVSSLRKNKGKCKEGEDDLDSLKSKVNCLATLDIFAGCGGLSEGLQKSGVCTTKWAIEYEEAAGDAFKLNHPESLMFINNCNVILKAIMDKTGDADDCISTPEAAELAAKLSEEEIKNLPLPGQVDFINGGPPCQGFSGMNRFNQSSWSKVQCEMILAFLSFADYYRPKYFLLENVRTFVSFNKGQTFRLAIASLLDMGYQVRFGILEAGAYGVPQSRKRAFIWAASPEETLPEWPEPMHVFAAPELKIALPENKYYAAVRSTQTGAPFRSITVRDTIGDLPMVSNGASRTSIEYQMDPISWFQKKIRANMMVLTDHISKEMNELNLIRCQRIPKRRGADWQDLPDEKVKLSSGQLVDLIPWCLPNTAKRHNQWKGLFGRLDWEGSFPTSITDPQPMGKVGMCFHPDQHRIVTVRECARSQGFPDSYQFYGNILHKHQQIGNAVPPPLAYALGMKLKEALESKGCM

SEQ ID NO: 37 - DNA, 5097 bp, Daucus carota
  misc_feature (0)...(0): AF007807.1; GI2895086;  Met1-type cytosine DNA-methyltransferase mRNA, complete cds
atcgatttccccgaagacccgaatcaaaccgggtcgggtccattgctttatgaaattgaaccgccaaaatgtatgggcgggaaaggacaattaaaaaatatgtttgcgcggttttttgttcttttccaaaatttgcagacgttttggggataaataagaggacccagatcgataaagatacaagatagtcaaaagggtcctataattcgtggatttttagttcagagtttgaattttttggttttgggttcttgaaatcttggtttctggggtctttgtttgatttgcttaatgggatcttcagctgttgttgatgctccagctctcgatgcaggtcttgaaacgaagaaaaataagcgaaagaatgcagattgtgattctgagaagacagcagtaagtggccaaaagaaacagagagcacatgccttaaagagtagtgagacacctgttggctcccgtaaaatgccaaagcgtgctgctgcttgtgcagatttcaaagagaaatctattcaaatatctaagaaatcttcaatcattgaaacaaaaaaggaccgttctgtagatgaagaggaagtagctgttcggttaacggctggacaagaagatggtcggccatgtaggaggctaactgactttatattccataattctgatggcataccgcaggcctttgaaatgttggaagttgatgatttatatatctctggcctgattttgcctcttgaggacagctcccaaaaggaagcatgtagcatcaaatgtgaagggtttggacgaattgagaactgggctctatctggctatgaagaaggggttccaacaatatgggtctcaactgatgttgcagattatgattgtgtcaaaccatcagctagttacaagaagcactatgaacatttatttgccaaagctactgcttgtgttgaggtgtacaagaaactgtcaaaatcttcaggtggaaatcctgatctgagtttggatgagttgcttgctggggttgttcgtggactgagtggtatgaaatgcttttctcgtagtgtatccatcaaagatttcattatatctcagggtgactttatttacaatcaacttgttggcttggatgagacatctaagaaaactgatcagcaatttcttgagctaccagtccttatagctttaagagaagaaagtagcaagcatggagacccttctatcggaaaggttgcatctactaatggaacattaacaattggtccaaaaattaaagacggtgagaacaaaaaggattctgcaacagaggaagatgagggtgtaaaagtggcaagattgttgcaggaagaagagttctggaactcaatgaagcagaaaaaaggccggggatcaagcacttcttctaacaaatattacataaaaattaatgaggatgagattgctaatgactatcctctaccagcatattacaagacagctaaccaagaaacggatgaatatataatttttgatggcggtgctgatgcgtgttatactgatgatttgcctcgaagtatgcttcataactgggcattgtacaactctgactcgaggctcatttccttggagctccttccaatgaaagggtgtgctgatattgatgtcactatatttggatcaggggtgatgactgaggatgatggaactggattcaatcttgatggtgacacgtctcaatcttcctcagctggattggggacagcaaatgttgatgggatcccaatatacctgagtgctataaaggaatggatgattgaatttggatcctcaatggtttttatatcaattcgcacagatatggcctggtataggcttggtaagccatcaaaacagtatgcatcgtggtatgaaccagttcttaaaacggccagggtcgctataagtattattacattattaaaggagcaggccagggtttctcgtctttcttttatggatgtcattaaaagagtttcggagtttgaaaagggtcatcctgcttacatatcatctgttccggcagctgttgagagatatgtagttgtgcatggacaaataattttgcagcagttcttagaatttcctgatgagaagattaaaaagtctgcatttgtgattggtctcacaaacaaaatggaagaaaggcaccacactaaatggcttatgaagaagaagaagttattgcagagggatgaaccaaacttaaatcccagagcagccctagcccctgtagtgtctaaaaggaaggctatgcaggcaacaactacacgactaatcaacagaatctggggtgagttttattcgaactactctccagaagatatgaaagagggaataactggtgaagataaggaggaagaagaacctgaagagcaagaggaaattgaggaggaagaggagaaggaaacattgactgctttagaaaaaactcctacacccacctcaacgccaagaaaaacaaaatcaattcctaaagtgaaggacataaggtggaaccgtaaatctgttggtgaaacattaagtggtgaagctctatacaaacaagcaatagtttatggaactgaaattgcagttgggggtgctgttctggtggatgacgaatctgcccaacttccagccatctattacgtggagtacatgtttgaaactttgaatggcataaaaatgcttcatgggagaatgttgcaacaaggatccctaacaatactcgggaatacagcaaatgaatgtgaagtatttctcacgaatgattgtatggattttgaattagcggatgttaaaaaagctgttgtagaaattcggtcaaggccttggggacaccagtacagaaaagtgaatgcaaatgctgataaaatctatagagcaggagttgaggagaggaaaaagaatggattggaaactgaatactattgcaaaagcttgtattgtccagataaaggtgcttttcttagccttcctcttaatagtatgggtctgggttcaggcatatgcagctcttgcaaattagataaagatctcactgaaaaagaaaaatttgtagtccactcagacaagacaagttttgtgttcaacggaactgaatattctattcatgattttctctacgtgagtcctcagcaatttagtacagaaagggtagggaatgaaaccttcaagggtggaagaaatgtgggattaaaagcttatgctatatgtcaactactcgaaattattgtccccaaggcacccaaacaagctgagccacattctactgagattaaggtaaggagattttaccggccagaagacatttcagatgagaaggcatactgctctgacattcgagaggtttattacagcgaagaaacacatacaattgatgccgagacagttgaagggagatgtgaagtgaggaaaaagaatgatcttccatcatgcgatgcgcctactatttttgatcatgtattcttttgcgaatatctgtacgatcctgctaaaggatctctcaaacagttgccaccaaatatcaaattgaggtattcagctgtgaagggtgcacatgtttcttctcttagaaagaacaagggtaagtgtaaggaaggggaggatgatttagattctctgaaatcaaaagtaaactgtttggcaaccttagacatctttgctggttgcggaggcctttcagaaggattgcagaaatccggtgtttgtacaacgaagtgggcaattgagtatgaagaggctgctggagatgcatttaagcttaaccatccagagtcgttgatgtttatcaataattgcaatgttattttaaaggctatcatggataagactggagatgcagatgattgtatttcaaccccagaggctgcagaattagctgcaaaattaagtgaggaggaaataaagaatttgccgctgccaggacaagtggattttattaatggagggcccccatgtcagggattttctggaatgaatagatttaaccaaagcagctggagtaaagtccagtgtgagatgattttggcgttcttatcctttgctgattattatcgaccaaagtattttcttcttgagaatgtcaggacttttgtgtccttcaacaagggacagacatttcgtctagctatagcttcacttcttgatatgggttaccaggttcggtttggtatacttgaggctggagcatatggagttcctcagtctaggaagcgagcatttatctgggcagcatctcctgaagaaactctcccagagtggccagagcctatgcatgtctttgctgcaccagagctaaaaattgcattaccagaaaacaagtactatgctgctgtccggagtactcaaactggggcaccatttagatcaatcactgttagggatacaataggagatcttccgatggttagcaatggggcatctaggacaagtatagagtatcaaatggatcctatctcctggttccaaaagaaaatccgtgcaaacatgatggtcttgacagatcacatatcaaaagaaatgaatgaactcaatctcattcgctgtcaaagaatccctaagcggcgaggtgctgattggcaagaccttcctgatgaaaaggtcaagctgtcttccgggcaattagttgacttgataccttggtgccttccaaatacagccaagaggcacaaccagtggaaggggctgttcggaaggttggactgggagggaagttttccaacttctatcactgacccccaaccaatgggaaaggtcggaatgtgcttccatcctgatcagcacaggattgtaacagtccgagagtgtgctcgttctcaaggcttcccagatagctaccagttttatggtaacattctacacaagcaccaacaaattggaaacgctgttcctcctcctctggcgtatgcactggggatgaaactcaaagaagccttagagagtaaggggtgcatgtagtttctcactcacttgcctcgctagtctgattgaactgatgcaagcaatttgtaaattaaaatctactgtttagtcgtcgtttcgtgcttgcaatagaaagcaactagaattgtcataggtctttcgaaacattggatcaatagaaagcaactagaattgttgtaggtc

SEQ ID NO: 38 - protein, 1559 aa, Lycopersicon esculentum
  PEPTIDE (0)...(0): gi | 2887280 | emb | CAA05207.1 | DNA (cytosine-5)-methyltransferase
MASPQPNSESVLELPNNDKSGHKKNKRKQDSVSKRKASATGKKEKKQAVSETIEEPTAGRKRPKRAAACSDFKEKSVHLSKKSSVIETKKDHCVDEEDVAIRLTAGLQESQRPCRRLTDFVFHNSEGIPQPFGMSEVDDLFISGLILPLEDSLDKVKAKGIRCEGFGRIEEWAISGYEDGTPVIWISTETADYDCLKPSGSYKKFYDHFLAKATACVEVYKKLSKSSGGNPDLSLDELLAGVVRAMTGIKCFSGGVSIRDFVITQGGFIYKELIGLDDTSKKTDQLFVELPVLASLRDESSKHETLAQPETISSGNGLRIGPKAGNGGDKIVESGLANGPAPEDEDLKLAKLLHEEEYWCSLKQKKDRNTSSSSSKIYIKINEDEIASDYPLPAYYKTSNEETDEYIVFDSGVETYHIDELPRSMLHNWALYNSDSRLISLELLPMKACADIDVTIFGSGVMTADDGSGYNFDTDANHSSSGGSRSAEIDGMPIYLSAIKEWMIEFGSSMIFISIRTDMAWYRLGKPLKQYAPWYEPVIKTARLAVSIITLLKEQNRVARLSFGEVIKRVSEFKKDHPAYISSNVDAVERYVVVHGQIILQQFSEFPDVSIRNCAFAVGLSRKMEERHHTKWVIKKKKVMQRLEQNLNPRASMAPSVKRKAMQATTTRLINRIWGEYYSNYSPEVSKEVADCEVKDDEEPDEQEENEEDDVPERNLDVPEKAHTPSSTRRHIKSRSDSKEINWDGESIGKTASGEQLFKKARVHGHEIAVGDSVLVEHDEPDELGCIYFVEYMFEKLDGSKMLHGKMMQRGSDTVLGNAANEREVFLINECMNLQLGDVKESIAVNIRMMPWGHQHRNTNADKLETAKAEDRKRKGLPTEFYCKSFYRPEKGAFFRLPFDKMGLGNGLCYSCELQQTDQEKESFKFDMSKSSFVYLGTEYSVDDFVYVSPDHFTAERGGNGTFKAGRNVGLMAYVVCQLLEIVGPKGSKQAKVDSTNVKVRRFFRPEDISSDKAYSSDIREIYYSEDIHTVPVEIIKGKCEVRKKYDISSEDVPAMFDHIFFCEYLYDPLNGSLKKLPAQINLILSKIKLDDATSRKRKGKGKEGVDEVGELNETSPQNRLSTLDIFAGCGGLSEGLQHSGVTDTNWAIEYEAPAGDAFRLNHPKTKVFIHNCNVILRAVMQKCGDSDDCISTPEASELAAAMDESELNSLPLPGQVDFINGGPPCQGFSGMNRFNQSTWSKVQCEMILAFLSFADYYRPKFFLLENVRNFVSFNQKQTFRLTVASLLEMGYQVRFGILEAGAYGVPQSRKRAFIWAGSPEEVLPEWPEPMHVFAVPELKIALSETSYYAAVRSTASGAPFRSLTVRDTIGDLPVVGNGASKTCIEYQGDPVSWFQKKIRGSSITLSDHISKEMNELNLIRCQRIPKRPGADWRDLEDEKVKLSNGQLVDLIPWCLPNTAKRHNQWKGLFGRLDWDGNFPTSITDPQPMGKVGMCFHPDQDRIVTVRECARSQGFPDSYQFAGNILHKHRQIGNAVPPPLAYALGRKLKEAVESKNRLT

SEQ ID NO: 39 - DNA, 5377 bp, Lycopersicon esculentum
  misc_feature (0)...(0): AJ002140.1; GI2887279; mRNA for DNA (cytosine-5)-methyltransferase
cccgcccaaatccccccaaaaacctatctcatttgtcctcttcctgttggagaactcagcaacagcacaccccatctccctcaacttctccgccgcaccagcttctactctccatttccgccgaaaaatcacctttcaccggcaaagcagcagcctctggtccctcctttatcttctcccttcgctgctctggccaccatcgctggtcgctggcgagaactacaacgaaaatcccttcgcctccgctctcctctctccctcttccgccgccctgctcctcacttctcacttctccattgaagtcgacggacgggataacggcagcgacgactgctccgcccagctactgtggcgaagtagcagcaagctgtgaccagcaaactcggcaaactccggccaaagcagcgataacaactcaggccagcagtggggcagcaacgactgctctgcccagcagctttggcgacggcaagctgtgaccagcaaactccggcgaagcagcgataacaactcaggccagcagtggggcagcaactccggttggtgaggatggcgtcaccccaacctaattcggagtcggtattagaacttccgaacaacgacaaatctggacacaaaaagaacaaacgcaaacaagattctgtgtcaaaaaggaaggcatctgcaactggtaagaaggaaaagaaacaggctgtttctgaaactattgaggagcccactgctggacgtaaaaggcctaagcgagctgctgcctgttcagattttaaagagaaatctgtgcatttatcaaaaaagtcttcagtcattgaaacaaagaaggaccattgtgtagacgaagaggatgtagctattaggttaactgcgggtctgcaagagtctcaacgaccctgtagaagattaacggattttgtttttcataactcagaaggaataccacaaccgtttggaatgtctgaggttgatgatctgtttatcagtggcctcattttaccacttgaggacagtcttgacaaagtaaaagcaaaaggaattagatgtgaaggctttgggcgtattgaagaatgggctatctctggctatgaagatggaactcctgtcatatggatctcaactgagacagctgattatgattgtttaaaaccctcaggtagttataagaagttttatgaccacttcttggccaaggcgacggcttgcgttgaggtttataagaagctttcaaagtcatctggagggaatcctgatttaagtcttgacgagttgcttgcaggggttgtccgagcgatgactggcataaaatgcttttcaggtggagtatccatcagggactttgtcatcactcagggcgggttcatatataaggaacttattggtctggatgatacatcaaagaagactgatcaactttttgttgagctacctgtcctagcttcccttagagatgaaagcagcaagcacgagacacttgcacaaccagagactatatcatctggtaatggtctacgtattggcccaaaagcaggaaatggaggagacaagatagttgaatctggtttggccaatggtccagcgccagaagatgaagatctaaaattggctaaattgttgcatgaagaggagtattggtgctccttgaagcagaagaaagaccgtaatacatcttcctcatccagcaaaatatacatcaagatcaatgaggatgagattgcaagtgattatcctttacctgcatattacaaaacatctaatgaagagactgatgagtatattgtctttgacagtggggttgaaacataccatattgatgagttgcctcgcagcatgcttcataattgggcattatacaactcggactcaaggctaatatctttagaactgctgccaatgaaagcttgtgctgatattgatgtaaccatttttgggtctggagtgatgactgctgatgatgggtctggctacaattttgacacagatgctaatcattcctcttcaggtggttctagatcagctgaaattgatggaatgccaatttacctgagtgctataaaagaatggatgattgagtttgggtcctcaatgatctttatatcaattcggactgatatggcctggtataggcttgggaagccattgaaacagtatgctccttggtacgaaccagtcataaagactgcaagattggcagtgagcatcattactttgttaaaggaacagaatcgtgtggctagactttcttttggagaagttattaaaagggtttcagagttcaagaaagaccatcctgcttatatatcatctaatgtagatgcagtggaaaggtatgtggttgtacatgggcaaattattctccagcagttttctgaatttcctgatgtaagcattaggaattgtgcatttgcggttggtctctcaaggaaaatggaagagaggcaccatacaaaatgggtgattaagaagaagaaggtgatgcagagactggaacagaacttaaatcctagagcatctatggcgccatctgtaaaaaggaaagctatgcaggctactacaacaaggctaatcaacagaatctggggggaatactattccaattactcacccgaggtgtcaaaggaggtggctgattgtgaggtgaaggatgatgaagaaccagatgagcaagaggaaaatgaagaggatgatgttccggagaggaacttggatgttccagagaaagctcatacaccttcttctacaagaaggcatattaagtcacgttctgacagcaaagaaataaactgggatggggaatccataggtaaaacagcttctggtgaacagttgtttaaaaaagctagagttcatggacatgagatagctgttggagattcagttctagtggaacatgatgaaccagatgagcttggttgtatttactttgttgaatacatgtttgaaaaattggatggtagcaaaatgcttcatggaaaaatgatgcaacgaggatctgacactgtacttggaaatgcagctaatgagagagaggtatttttgatcaatgaatgcatgaatctgcaactaggagatgtcaaagaaagtatagctgtcaatatcagaatgatgccttggggacaccagcatagaaacacgaatgctgataaacttgaaacagcaaaagcagaagacagaaagaggaagggattgccgacggaattttactgcaaaagcttttatcgccctgaaaaaggtgcttttttcagactcccgtttgataagatgggccttggtaatggtttatgctactcttgtgagttgcagcaaactgatcaggaaaaggaatcctttaagtttgatatgtccaaatccagttttgtatatctggggactgagtattcagttgatgactttgtttatgtaagccccgatcactttactgcagaaagagggggaaatggaactttcaaagccggaagaaatgtggggttgatggcctatgtagtatgtcaattactagaaattgttggacctaagggatctaaacaagctaaagtagattctacaaatgttaaagtcagaagattcttcagaccagaggatatatcttcagataaggcatactcttctgatatccgggagatctattacagtgaagatatacatacagttcctgtggaaataatcaaaggaaaatgtgaagtgaggaagaagtatgatatttcctctgaagatgtccctgccatgttcgaccatattttcttttgtgaatatttgtatgatccattgaatggatcccttaagaagttaccagctcagataaacctgatattgtcaaaaattaagctagatgacgcaacatctaggaagaggaaggggaagggaaaagaaggagtggatgaagttggggaactaaatgaaacttctccacagaatcgtttgtccacattagatatctttgctggttgtggtggcttgtctgaggggttgcagcattcgggtgtcacagatacaaattgggcaattgaatacgaagcgcctgctggagatgcatttagacttaatcatccaaagacaaaggtgttcatacataattgcaatgtgattttgagggctgtcatgcagaagtgtggagattctgatgactgtatctcaactccagaggcttctgaattagctgcagcaatggatgagagcgaactgaatagtttgccactgcctggacaagttgatttcattaatggaggccctccttgtcaggggttttctggaatgaatagatttaatcagagcacctggagtaaagtacagtgtgagatgattctggcatttttatcctttgctgattattatcggcccaagttttttctcttggagaatgttaggaattttgtttcgttcaaccaaaaacaaacatttcgcttaactgttgcttcccttcttgagatgggttatcaggttaggtttggtatccttgaagccggagcgtatggagttcctcagtctaggaagagagcatttatctgggctggctccccagaggaggttcttccagagtggccagaaccaatgcatgtttttgctgtcccagaattaaaaatcgcattatctgaaacttcatactatgcagctgtgaggagtactgctagtggagctccattccgttcacttactgtcagagacacaattggagatcttcctgttgttggcaatggggcaagcaagacttgcatagagtatcaaggtgatccagtatcctggttccaaaagaaaatccggggcagctcaataacattatctgatcacatttcaaaagagatgaatgagcttaacctaatcaggtgccaaagaatccccaagcggccaggagctgattggcgtgaccttgaagatgaaaaggttaaactatctaatggtcaactagttgatttgattccatggtgcctgcctaacactgctaagcggcacaaccagtggaaggggctctttggaaggttggattgggatgggaacttccccacttctattactgatccccagccgatgggcaaggtggggatgtgctttcatccagatcaagacaggattgttacagttcgtgaatgtgcacgttctcaaggtttcccagacagctaccaatttgctggtaacatcttgcacaagcacaggcaaataggaaatgctgttccacctcctttggcatatgcgcttggaagaaaactcaaagaagctgttgagagcaaaaataggctcacttagaacttttttaagctgtgaattttacatgcatgtcaattaccattcacattgccaaattatatcagttactcatttattaaatttgcagtttcacctataaccctctatttagaggttgggttcaaacaaaattgattaaaacattact

SEQ ID NO: 40 - protein, 1556 aa, Nicotiana tabacum
  PEPTIDE (0)...(0): gi | 7288140 | dbj | BAA92852.1 |  DNA (cytosine-5-)-methyltransferase
MAYSFFHFFAGYSGHKKEKSKRDSVSKRKAPATDKKEKKQPVSEAIEEPTAARKRPKRAAACSNFKEKNVHLSKNSAVIETKKDQCVEEEVLAIRLTAGLQDSQRPCRRLTDFIFHNLEGIPQPFEMSEVDDLFITGLILPLEDNNDKEKAKGIRCEGFGRIEEWAISGYEDGTPIIWISTETADYDCKKPSGGYKKFYDHFFAKATACIEVYKKLSKSSGGNPDLSLDGLLAGVVRAMSGLKCFSGGVSIRDFLISQGEFVYKQLIGQDDTSKKTDQLFVELPVLASLRDESSNQEMLSQPEPLSFGRTLTIGPKVGKGEGKRDQSDLTTGPEQEEEDLKLAKLLHEQEYWHSLNQKTSRSTSSSSSKFYIKINEDEIASDYPLPAYYKTCNEETDEYIVFDSGVDTYYIDDLPRSMLHNWALYNSDSRLISSELLPMKPCADIDVTIFGSGVMTADDGSGYNVDADANNSSSGGSGSAEIDGMPIYLSAIKEWMIEFGSSMIFISIRTDMAWYRLGKPSKQYAPWYEPVLKTAKLAVSIITLLKEQSRCARLSFGDVIKRVSEFKKHHPAYISSNTDVVERYVVVHGQIILQQFSEFPDESIRKCAFVIGLSRKMEERHHTKWLIKKKKVVQRHEQNLNPRASMAPSVKRKAMQATTTRLINRIWGEYYSNYSPETSKEVVACEVKDDEEVDEQEENDEDDAQEENLEVSEKTHTPCSTRRHIKSRSDSKEINWDGESIGKTASGELLFKKPRIHGNEIAVGDSVLVEHDEPDELPSIYFVEYMFEKLDGSKMLHGRMMQRGSETVLGNAANEREVFLINECMDLQLGDVKESVVVSIRMMPWGHQHRKANAYVDKLDRAKAEDRKKKGLPSEFYCKSFYQPDRGAFFRLPFDKMGLGNGLCYSCELQQIDQEKESFKLDMSNSSFVYLGTEYSIDDFVYIHPDHFAVERGGSGTFKAGRNVGLMAYVVCQLIEISGPKGSKQAKVDSTNVKVRRFFRPEDISSDKAYSSDIREIYYSEEIHTVPVETIEGKCEVRKKYDIPSEDVPATFDHVFFCEYLYDPLNGSLKQLPAQVKLRFSRVKLDDAASRKRKGKGKEGEDELRVGQLNVASQQNRLATLDIFAGCGGLSEGLQRSGVSDTKWAIEYEEPAGDAFKLNHPEAKVFIQNCNVILRAVMQKCGDAENCISTSEASELAAAMDENELNSLPLPGQVDFINGGPPCQGFSGMNRFNQSTWSKVQCEMILAFLSFADYYRPKFFLLENVRNFVSFNQKQTFRLTVASLLEMGYQVRFGILEAGAFGVPQSRKRAFIWAASPEEILPEWPEPMHVFGVPELKITLSETCHYAAVRSTASGAPFRSLTVRDTIGDLPAVGNGASKTCIEYQVDPISWFQRKIRGNSITLSDHITKEMNELNLIRCQRIPKRPGADWRDLPDEKVKLCNGQLVDLIPWCLPNTAKRHNQWKGLFGRLDWDGNFPTSFTDPQPMGKVGMCFHPDQDRIVTVRECARSQGFPDSYQFAGNILHKHRQIGNAVPPPLAYALGRKLKEAVESKKRST

SEQ ID NO: 41 - DNA, 4822 bp, Nicotiana tabacum
  misc_feature (0)...(0): AB030726.1; GI7288139; mRNA for DNA (cytosine-5-)-methyltransferase, complete cds
atggcttattcttttttccatttttttgctggttattcaggacacaaaaaggagaaaagcaaacgagattctgtgtcaaaaaggaaggcacctgcaactgacaagaaggaaaagaaacagcctgtttctgaagctattgaggagcccactgctgcacgcaaaaggcccaagcgagctgctgcttgttcaaattttaaagagaaaaatgttcatttatcaaaaaattctgcagtcattgaaacaaagaaggaccaatgcgtagaggaagaggttttggctattcggttaactgcgggtctacaggattctcagcgaccctgtagaagactaacagattttatctttcataatttggaaggaataccacaaccttttgaaatgtctgaagttgatgatctgtttattactggtctcattttaccacttgaggacaataatgacaaagaaaaagcaaaaggaattagatgtgaaggctttgggcgtatagaagaatgggctatctctggctatgaagatggaactcctatcatatggatctcaacagagacagctgattatgattgtaaaaaaccctcaggtggctataagaagttttatgaccacttcttcgccaaagctacagcctgcattgaggtttacaaaaagctgtcgaaatcttctggaggaaatcctgatttaagccttgatgggttgcttgcaggggttgtccgagcaatgagtggtttaaaatgcttttcgggtggtgtatcaatcagggactttctcatttctcagggagagtttgtctataagcaacttatcggtcaggacgatacatcaaagaagactgatcagctttttgttgagttacctgtcctggcttcccttagagatgaaagcagcaatcaggaaatgctttcacaaccagagcctttatcatttggtaggactctaactataggtccaaaagtaggcaaaggagaaggcaagagagatcaatctgatttaaccactggtccagaacaagaagaggaagatctgaaattggccaaactgttacatgaacaggagtactggcactccttgaaccagaagacaagccgtagtacatcttcctcatctagcaaattttacatcaagatcaatgaggatgagattgcaagtgattatcctttacctgcatattacaagacatgtaatgaagagaccgatgagtatatcgtctttgacagtggggttgatacatactatattgatgacttgcctcgcagtatgcttcataattgggcattgtacaactcagactcaagactaatttcttcagagctcctgcctatgaaaccatgcgctgatattgatgtaaccatatttgggtctggagtgatgactgctgatgatggatctggatacaatgttgatgctgatgctaataactcctcttcaggtggttctggatcagctgagattgatggaatgccaatttatttgagtgcaataaaagaatggatgattgagtttgggtcctcgatgatctttatatctattcggactgatatggcctggtataggcttgggaagccatcaaaacagtatgctccttggtatgaaccagtcctaaagactgcgaagttggcagtgagcattattactttgttaaaggaacaaagtcgttgtgctagactttcttttggagatgtcattaaaagggtttcagagttcaagaaacaccatcctgcttatatatcatctaatacagatgtggtggaaagatatgtggttgtacatggacagattattctgcagcagttttcagaatttcctgatgaaagcattaggaaatgtgcatttgtgattggcctctcaaggaaaatggaggagaggcaccatacaaaatggttgattaagaagaagaaggttgtgcagagacatgaacagaacttaaatcctagagcatctatggcgccatctgtaaaaaggaaagctatgcaggctactacaacaagactaatcaacagaatctggggggagtactattccaattactcacctgagacgtcaaaggaggttgttgcttgtgaggtgaaggatgatgaagaagtagatgagcaggaggaaaatgacgaggatgatgctcaagaggagaacttggaagtttcagagaaaactcatacaccttgctctacaagaaggcatattaagtcacgttctgacagcaaagaaataaactgggatggggaatccataggtaaaacagcgtctggtgaactgttgtttaaaaagcctagaattcatggaaatgagattgctgttggagattcagttctggtggaacatgatgaaccagatgaacttccttctatttactttgtcgaatacatgtttgaaaaattggatggtagcaaaatgctccatggaagaatgatgcaacggggatctgaaactgtacttggaaatgcagctaatgaaagagaggtatttttgatcaatgaatgcatggatttgcaactaggagatgtcaaagaaagtgtagttgtcagtatcaggatgatgccatggggacatcagcatagaaaagcgaatgcttatgttgataaacttgatagagcaaaggcagaagacaggaagaagaagggattgccatccgaattttattgcaaaagcttttatcagcctgacagaggtgctttcttcagacttccgtttgataagatgggtcttggtaatggcttatgttactcctgtgagttgcagcaaattgatcaggaaaaggaatcttttaagttggatatgtccaactccagttttgtatatctggggactgagtattcaattgatgactttgtttatatacaccctgatcactttgctgtagaaagagggggaagtggaactttcaaagctgggagaaatgtggggttgatggcctatgtagtgtgtcaactaatagagatttctggccccaagggatctaaacaagctaaagtagattctaccaacgtcaaagtcaggagattcttcagaccagaggacatttcttcagataaggcatactcttctgatattcgggagatctactatagtgaggagatacatacagttccggtagaaacaattgaaggtaaatgtgaagtgaggaagaagtatgatattccgtctgaagatgtccctgccacctttgaccatgttttcttttgtgaatatttgtatgatccattgaatggatccctcaaacagttaccagctcaggtaaagctgagattctcaagagttaaactagatgatgctgcatctaggaagagaaagggaaaaggcaaggaaggagaggatgaactgagagttgggcaactaaatgtagcttctcaacagaatcgtttggccacactagatatctttgctggttgtggtggcctgtctgaggggttgcagcgttcgggtgtctcagatacaaaatgggcaattgaatatgaagagcctgctggagatgcgtttaaacttaatcatccagaggcaaaggtgttcatacagaattgcaatgtgattctgagggctgtcatgcaaaagtgtggagatgctgagaactgtatctcaacctcagaggcttctgaattagctgcagcaatggatgagaacgaactgaatagtttgccactgccaggacaagtggacttcataaatggaggccctccttgtcaggggttttctggaatgaatagatttaatcagagcacctggagtaaagttcagtgcgagatgattctggcatttttatcctttgctgattattatcggcctaagttctttctcttggagaatgttaggaattttgtgtcgttcaaccaaaaacaaacatttcgcttaactgttgcttcccttcttgagatgggttatcaggtgaggtttggtatccttgaagctggagcgtttggagttcctcagtctaggaagagagcatttatctgggctgcttccccagaggagattcttccagagtggccagaaccaatgcatgtatttggtgtcccagaattaaaaatcacattatctgaaacttgtcactatgcagctgtgaggagtactgctagtggagctccattccgttcgcttactgtcagagacacaattggagatcttcctgctgttggcaacggagcatccaagacctgtatagagtatcaagttgacccgatatcctggttccaaaggaaaattcggggcaactcaataacattatccgatcacattacgaaagagatgaacgagcttaacctaatcaggtgccaaagaattcctaagcggccaggagccgactggcgtgaccttccggatgaaaaggttaaactatgtaatggtcaactggttgatttgattccgtggtgcctgcctaacactgctaagaggcacaaccagtggaaggggctctttgggaggttggattgggatgggaacttccccacttcctttactgacccccagccgatgggtaaggtggggatgtgttttcatcccgaccaagacaggattgttacagttcgtgaatgtgcgcgttctcaaggtttcccagatagctatcaatttgctggtaacattttgcacaagcacaggcaaataggaaatgctgttccacctcctttggcatatgcactgggaaggaaacttaaggaagctgttgagagcaagaagaggtccacttagaagtttgtaaattttgtggaacaagagatgagtggtcatactgcacctgaatttaagctttcaaatttaaatgtcaaacagcatgattcacatgtcaattttctgttgtacaagatagcttattgcagaatcaatgttacataaaaaaaaa

SEQ ID NO: 42 - protein, 152 aa, Triticum aestivum
  PEPTIDE (0)...(0): Ceres Clone890048; Met1 homolog
DHISKEMNELNLIRCKHIPKRPGCDWHDLPDEKVKLSSGQMVDLIPWCLPNTAKRHNQWKGLYGRLDWEGNFPTSVTDPQPMGKVGMCFHPDQDRIITVRECARSQGFPDSYQFSGTIQSKHRQIGNAVPPPLAFALGRKLXXAVXGXHQQA

SEQ ID NO: 43 - DNA, 457 bp, Triticum aestivum
  misc_feature (0)...(0): Ceres Clone890048; Met1 homolog
cgatcacatatctaaggagatgaatgaattaaatctcataagatgcaaacatattcccaaacgacctggttgtgactggcatgacctgccagatgagaaggtgaagctatcttctgggcaaatggtggacctgataccttggtgcttgcctaacaccgctaaaaggcacaatcagtggaagggtctgtatgggaggttagattgggagggcaatttccccacgtctgtgactgatcctcagccgatgggcaaggttggcatgtgcttccaccctgaccaggataggattatcacggtccgcgaatgtgcgcgatctcagggctttcctgacagctaccagttttcgggcaccattcagagcaagcacaggcagattggcaatgctgtgccaccccctcttgcctttgcgcttgggaggaagctgamtsaagccgttsatgggaakcaccagcaggcc

SEQ ID NO: 44 - protein, 1525 aa, Zea mays
  PEPTIDE (0)...(0): gi | 3132825 | gb | AAC16389.1 | putative cytosine-5 DNA methyltransferase
MQSKATKEGRGIHRKQQAGEWISGYNRRGASWSRKSDGHVTRKRPRRSAACSDFKEKSIRLSEKKSVVMVKKNRMEEEEVDAVNLTKLGPEDPPPCRKLIDFILHDAEGNPQPFEMSEIDDFFITALIMPMDDDLEKERERGVRCEGFGRIEDWNISGYDEGTPVIWVSTDVADYECVKPSTNYKSYFDHFYEKAQVCVEVFKKLAKSVGGNPNQGLDELLASVVRSTNAMKGYSGTMSKDLVISIGEFVYNQLVGLDETSNNDDEKFATLPVLLSLRDQCRSRVELTKLPSNFSNTSLKIKDSECDETAEDDDDAKLARLLQQEEEWKMMKKQRGRRGTPSQKNVYIKISEAEIANDYPLPAYYKPFSQEMDEYIFDSDDSIFSDDVPVRILNNWTLYNADSRLISLELIPMKSGAENDVVVFGSGFMRDDDGSCCSTAESVKSSSSSSKADQLDAGIPIYLSPIKEWIIEFGGSMICITIRTDVAWYKLRQPTKQYAPWCEPVLKTARLAVSIITLLKEQSRASKLSFADVIRKVAEFDKGNPAFISSNITLVERYIVVHGQIILQQFADFPDETIRRSAFVSGLLLKMEQRRHTKLVMKKKTQVMRGENLNPSAAMGPASRKKAMRATTTRLINRIWSDYYAHHFPEDSKEGDGNETKEIDDEQEENEDEDAEDEGQIEENISKTPPSTRSRKLLSQTCKEIRWEGETSGKTLSGETLYKCAYVRELRIPVGGTVALEDDSGDTVICFVEYMFQKVDGSKMVHGRILQKGSQTILGNAANEREVFLTNDCLEFKLDDIKELVMVDIQSRPWGHKYRKENSEADKVEQVKAEERKKKGQPMVYFCKSLYWPEKGAFFALSRDKMGLGSGLCSSCDNIEPDSDELKIFSKTSFVYRKVTYNVNEFLYIRPDFFAEDEDRATFKAGRNVGLKPYAVCQILSIPEGAGSKKLNPASANISARRFYRPDDISSAKAYASDIREVYYSEDVIDVPVDMIEGKCEVRKKNDLASSDLPVMFEHVFFCELIYDRASGALKQLPPNVRFMSMVQRTSALKKNKGKQICEPDQIDSGKWLDVPKENRLATLDIFAGCGGLSEGLQQAGVSFTKWAIEYEEPAGEAFNKNHPEAVVFVDNCNVILKAIMDKCGDTDDCVSTSEAAEQAAKLPEVNINNLPVPGEVEFINGGPPCQGFSGMNRFNQSPWSKVQCEMILAFLSFAEYFRPRFFLLENVRNFVSFNKGQTFRLAVASLLEMGYQVRFGILEAGAFGVAQSRKRAFIWAAAPGEMLPDWPEPMHVFASPELKITLPDGQYYAAARSTAGGAPFRAITVRDTIGDLPKVGNGASKLTLEYGGEPVSWFQKKIRGSMMVLNDHISKEMNELNLIRCQHIPKRPGCDWHDLPDEKVKLSNGQMADLIPWCLPNTAKRHNQWKGLYGRLDWEGNFPTSVTDPQPMGKVGMCFHPDQDRIITVRECARSQGFPDSYEFAGNIQNKHRQIGNAVPPPLAYALGRKLKEAVDKRQEASAGVPAP

SEQ ID NO: 45 - DNA, 7955 bp, Zea mays
  misc_feature (0)...(0): AF063403.1; GI3132824;  putative cytosine-5 DNA methyltransferase (ZMET1) gene, complete cds
ccctccactgctcctacctttaacgaagcagcctggcagcacataaactttcattttgaacttgttcaacccgctgctgtgtttatggatctttggcatcattgatggcattaaacttttgagtctggcacttactgatctccaccttgaaccaggacatttcttcatcccattttgcttcctttctgttctttgttgctttctcaaatcttccctaaacccaaccaaatttctttaaacaaaaacgtgtatatgtgcatttttagcccacacgcggattcgagaacaagctctatgagcatcttcctccctattgactgtcaaaaaaaagacggtgatgcatgacaccacctcaccttatcgaatcatgtctcccttgttctgttctccaaccatgctgcacacctgccatttgtcatatactcatcaaaattcatataaaacccccaatcgtatcaattccaatcccgtactagttaaaagataactatgtggagttgtcgcttcttcccgtaatgtagttaagttagagggccctggtgtggcgctcccgtcctgggtttgagccttggcattgcaccggtggtgcacccacctcatggctggtggcggtgcaaatggttctgtgaccaccaatgaagcgagtgcacatgaggttcttgcctgctttccgtggtttggtgggtccctatcttaatacagtcaaatgtacatctctccttgatcaaatttccccgttaacccatgtggattatgtggtattgagtcgtaaatccatagcaagtcaaaattcatcacaatccattccaatacactccaatccacatggaattggaataaccgaacaatgccttagttggaaatggagtcattccagtctcttacatctgacacaaatatctttcctgagttgtgacaaccagtgttacccagacatctgcgttccttttttttgcggagccagaaaacttgtcggtttccaagtggtgtacccccccccccccccccccaatttttttttgtcaaactggacacctgcacccgtaccggacacaaatacccgcactagcatgtgtcccatgtgacactggtaaagtatttggcattttgtgttcccatttaccctcccataatggtaatgtcagttgttgcagaatcttacgttttaagcaaatcatgtgaattggttaccgttttcctatacacatttcacatgaaccattgggattggtattgcaactatgataacagaggtatgctgagtgttcagtaaattcaaaccatttttgaggatctattttgtttctccaagggtacactggtagattaattacataggctctggcattccagtggcttatattattattttttctttctattcttggaatggtcggatattaaactgcctaccttttaaaatgtggtctcctgatgcaatattgtggctcatgtagttttaaatttaggaaagggaacactatttacaggctacaactccattttttaccactaatgacattttagaaaaaaaaatgaaggtatttctaaatgatcttttgtcttaaatattgtctttgttgctgcacttcacaggtctatatttttctagttactgatagcaagcattaacaatcttttgtcatttggtcagtatttattctgttccttaaatctagtcagtcaccctaaccttccttttttgttgattttgtgttttgtctgcatctctggccggtgtgtttttcttttctttctgttcacttttcagtactgctattttaacttttgttcccctatataggcatatatctgattgatatgctgaccaatgatttttcaggaacaagaagatgcagagcaaagccacaaaagaaggaagaggaatccacagaaaacaacaagctggagaatggatctctggatacaacagaagaggtgcatcatggagtcgaaaaagtgatggacatgttacccgcaagagaccaaggagatcagcggcctgttctgatttcaaagagaaatccatacgcttatccgaaaaaaaatctgttgtcatggtcaagaagaatcggatggaggaggaagaagtagatgctgtcaatctgacaaaacttggaccagaagatccaccaccttgccggaagttgatcgattttatcttgcatgatgcagaagggaacccacaaccctttgaaatgtcagaaattgatgacttctttataacagctcttatcatgcccatggatgatgatctagaaaaagagcgtgaaagaggagtacgctgtgaaggatttgggcgaattgaggactggaatatttctggttatgatgaaggtactcctgtaatctgggtgtcaactgatgttgctgactatgaatgtgtgaaaccatcaaccaattacaaatcttattttgaccacttctatgagaaggctcaggtgtgtgttgaagttttcaaaaagcttgcaaaatcagttggtgggaatcctaaccagggcctggatgaattgcttgctagtgttgttcggtcaaccaatgccatgaaaggatatagtggaaccatgagcaaagatttggtgatatccattggagaatttgtatacaatcaacttgttggtttggatgagacatcaaacaatgatgatgaaaagtttgctaccctgccagttcttctttctctaagagaccagtgcagatctagggtggaactgaccaagttgccctctaacttctcgaacacaagtctgaaaattaaggactcagagtgtgatgagacagcagaagacgatgatgatgcaaaattagctagattacttcaacaagaagaagaatggaaaatgatgaagaaacagaggggtaggcgtggaacaccatcccagaaaaatgtctacataaaaatcagtgaagctgagattgccaatgactatccccttcctgcatactataagccatttagccaggaaatggatgaatacatatttgatagtgatgacagcatattttctgatgatgtgccagttaggatactcaataactggacactgtacaatgcagattccaggcttatatctttggaattgatccctatgaaatcaggggcagaaaatgatgtggttgtctttggatctggtttcatgagagatgatgatggcagttgctgttctacagctgagtctgtgaaatcttcgtcttcctccagcaaagctgaccaactggatgcgggaatccctatttatttgagcccaatcaaagaatggattatagagtttggtggctcaatgatttgtataaccattcggactgatgtggcctggtaagtaccctcagctactttctttcagtacactgcttcattatgtggtcattaactgtgttcttaacagttgtgtcactgtatcctcttataccatttgaacatcacttttagctcttttaatctttgctccattacaacttacatttagagttttatttcaggtacaagctacgccaaccaacaaaacaatatgctccatggtgtgagcctgtactgaaaacagcaaggcttgctgttagcatcattaccctgttgaaagagcagagtcgtgcctcaaagctttcttttgctgatgtcataagaaaagtagctgaatttgacaaaggaaaccctgcatttatatcttcaaacatcacacttgttgagagatatattgtggtgcatggacagataatactccagcagtttgcagattttccagatgagactattcgtcggagtgcatttgtcagtggtcttttattgaagatggaacagaggaggcatacaaagttagttatgaagaaaaaaactcaagtgatgaggggagagaatctgaatccaagtgcagcaatgggtccagcatcgaggaaaaaagcaatgcgtgcaacaacaaccaggctcatcaacagaatctggagtgattactatgcacatcatttccctgaagattccaaggagggagatggaaatgaaacaaaagaaattgatgatgaacaagaagaaaatgaagatgaggatgctgaagatgaaggacagattgaggagaacatctcaaagactcctccatcaacacggtcccggaagttgctatcacaaacttgtaaggaaatcagatgggaaggtgaaacatctgggaaaacattgtctggagaaactctatataaatgtgcttatgttagggaactcagaatacctgttggtggaacagtggctctagaagatgattcaggagacacagtcatttgttttgttgagtacatgttccagaaagttgatggttcaaaaatggttcatgggaggattctgcaaaaagggtcacagacaattcttggcaatgcagcaaatgagagggaggttttcttaactaatgactgcttagaattcaaattagatgacatcaaggaattggtaatggttgatatccaatcaaggccttggggtcacaagtacagaaaagagaattctgaagctgataaagttgagcaggtcaaagcagaagagagaaagaaaaagggccagcccatggtatatttctgcaaaagcttgtactggcctgagaagggtgccttctttgccctctcccgagataaaatgggtcttggtagtggtttatgtagttcttgtgataatatagagccagattctgatgaattgaaaatattctcgaagaccagctttgtctacagaaaggttacatataatgtcaatgagtttttatacataagacctgatttttttgctgaagatgaggatcgtgcaaccttcaaggctggccgaaatgtgggtctaaagccctatgcagtttgtcaaatattgtccatccctgaaggggctggatctaaaaaactcaatccagcatcagcaaatatcagtgctagaagattttacagaccagatgacatttcatcagccaaagcctatgcatctgacatcagagaggtcatcttttttttctatcttgtatgcttgatttatctactccataacttcattgttactttttctcaaacatgtgagcaaatcctagagtcctgagaatggtcattcttgtttctttcttgttaactttagtttgttcgattcaggtctactatagtgaggatgtaattgatgtgcctgtggatatgatagagggaaaatgtgaggttagaaagaagaacgatcttgcaagttcagaccttccagtgatgtttgaacatgtatttttctgtgaacttatatatgaccgtgccagtggagctctcaagcaggttagctgtactgtactgaagttgctattctgattcattgagtggcagttttgatagtttcctgaatgtgtgttccatgtctggagcagttgcctccaaatgttaggtttatgtctatggtgcaaaggacaagtgcgttgaaaaagaacaaaggaaagcagatctgtgagcctgatcaaatagattcaggtaaatggttggatgtgcctaaagagaaccgtctagctactcttgacatttttgctggctgtggaggtttatcagaagggctgcagcaagctggtatgtattgttaacactgatgctgtataccatgaacatgaccaacaaataaaaaatttcctcattgttcaatgctgtaggtgtatcttttacaaaatgggcgattgaatacgaagagcctgctggtgaagcatttaataaaaatcatccagaggctgtggtctttgtagataactgcaatgtgattctaaagtaagtgcaaattgtttgatgccattattatattttttgttgttgaacagaaccaatatttttggtaatgcagggcaattatggataaatgtggggatactgatgattgtgtttcaacttctgaagctgctgaacaagcagcaaaacttccagaagtgaacattaataatcttccagtccctggcgaagttgaattcataaatggtggtcctccgtgtcaggtttgttattatctacagttctatgtataggccagaaaatcatcagtcacctgttcagttttgtcattcaaatgcttgaattgtttattcttttgttgtcagggattctctgggatgaatagattcaaccaaagcccatggagtaaagttcagtgtgagatgattctagcattcctctcattcgctgagtatttccgtcccagattctttctgttagaaaatgttcggaactttgtttccttcaacaaagggcagaccttccgtttggcagttgcatctcttctggagatgggataccaggtatttctgttaattcattatctgctaagacctatagcttacactttttatggtggtttaaatctgtatacttagaaattgtttgccatttggttaggtccggtttggaattctagaagcaggggcttttggtgttgcccagtccaggaaaagggcgtttatttgggctgctgcacctggagagatgcttcctgattggccagagccgatgcatgtgtttgctagccctgagctgaagataacactgcctgatggccaatactatgcagctgcaagaagcactgctggtggagcgcctttccgagcgattactgttagagatacaattggggatctgcctaaagtgggaaatggtgccagcaaactcacgcttgaggtaactggtgcttcttgatcatctatttttttcttttctttgagttatatgctaaatgagctactgattatcttgtgcagtatggaggtgagcccgtgtcttggttccagaagaagataagagggagtatgatggtactgaatgatcacatatctaaggagatgaatgagctgaacctaataaggtgtcaacacattccgaaacggccgggttgtgattggcatgacctaccggacgagaaggtaattttctgaaatctgttgttatattccttctgtccatggagcactgacccttggcccttgctattcttacaggttaagctgtcaaatgggcagatggctgacctgataccttggtgcctgcccaacacagccaagaggcacaatcagtggaaaggactgtacgggaggctggactgggaaggcaacttccccacatccgtcactgatccccagccaatgggcaaggtcggcatgtgcttccaccctgatcaagacaggatcatcacagtccgggaatgtgctcggtcacaggtaagctggtctacatccatttccatctgcaaaatgacaatgacactcctgtctaatatgatccaatctttgccgtgcagggctttcctgacagctatgaatttgcgggcaacatccagaacaagcaccggcagattggcaatgccgtgcccccgcctcttgcctatgcacttgggaggaagctcaaggaagccgttgacaagcgtcaggaagccagcgcaggcgtgcctgcaccatgagaagttttccttccatcaaaccatgacccatgaagctaagcgctgaggtcgtccttgaggaccagttaattttggttttatcagtcttaatggactcctgaatgtatatgttagagaagtgtcgattgttgattgttaccctgattcagggtagcggttatatctaaaaacttgagaaaatctagtgtactctagttgctatgtgttccattttgttgactctaaactttcaactagttttggtgattaatgacaacatgagattaacttaaattttgtagaggtatttaaattaggccactaatagtgactatttagtcgctcaatttttttgcccctaattatggaatttgttttttaaaggatgaacaacaagattaaatggattagttcaagtgtcgattcgggctaagactatccgtagcggttttttctaactttttctctatgtgccacctttatatcatgtcatactagcaattctaattaattggttaagggcatcctattacatcattgtggtagcattgttttgggt

SEQ ID NO: 46 - protein, 1522 aa, Oryza sativa
  PEPTIDE (0)...(0): gi | 18653391 | gb | AAL77415.1 | putative cytosine-5 DNA methyltransferase (japonica cultivar-group)
MDTCLYGTKRRRAKVHKEDEPVENENLESEFDVSKKESNGATEPGNEPVASKRPKRAAACSNFKEKSLDLSEKDSIITIKESRVEEKEIEAVNLTRTGPEDGQPCRKIIDFILHDGDGNLQPFEMSEVDDIFITALIMPLDDDLEKDRGKGICCSGFGRIENWAISGYDEGAAVIWVSTETSDYKCVKPASSYRSYFEHFSEKARVCVEVYKKLARSVGGNPQVDLEELIAGVVRSINSNRSFNGTVTKDFVISSGEFIYKQLIGLDHTAGNDDEMLATLPVLVALKDECKSRAGFTHLPAMPSNGTLRIKDGQDKGLTEDEDAKLARLLQEEEEWKMMKQRGKRGTSQKNIYIKICETEIANDYPLPAYYKPYNQEMDEYIFDSDIGMYSDDVPVRILDNWALYNSDSRLISLELIPMKAGAENDIVVFGSGFMREDDGSCCSTAELAQLHSSSSKSGREDPGVPIYLSPIKEWVVEFGGSMICITIRTDVAWYKLRQPTKQYAPWCEPVLKTARLAVSIITLLKEQSRASKLSFAEVIKKVAEFDSRHPAFISSKAPTVERYVVVHGQIILQQFADFPDESVKRCAFITGLLAKMEESRHTKLAIKKKSQQMRGENLNPSAKMGPILRKKLMRATTTMLISKIWGEYYATYFPGDTKEEDQNEPKEIDDDQEENEDNDAEEEVNVQDEKATRTPPSTRSRKSSADTRKEIKWEGQTAGKTVSGEVLYKCVIVQDLSISVGATVTTEDDSGETIMCFVEYMYEKLDGKNMIHGIILQEGSQTVLGNAANDREVFLTNDCLEFEASDIKELVTVNIQSLPWGHKYRKENSEAKRIEKAKAEERKRKGLPVEYICKSLYWPEKGGFFSLPYDKIGNGTGICSSCERKPVGNEFKLLSESSFVFENITYNIHDFLYIRPEFFSQGEGHETYKAGRNVGLKPYAVCHLLSVHGPAGSRKANPESTKVKVRRFYRPDDISSTKAYSSDIREVYYSEDIISVPVVMIEGKCEVRLKDDLPNSDLPAVVEHVFCCEYLYDPANGALKQLPPNVRLVTLTRKVPASKKNKGKQICDIELGGSDKPKDGQSENCLATLDIFAGCGGLSEGLQRSGLSLTKWAIEYEEPAGDAFGENHPEAAVFVENCNVILKAIMDKCGDSDDCISTSEAAERAAKLSEDKIKNLPVPGEVEFINGGPPCQGFSGMNRFNQSPWSKVQCEMILAFLSFAEYFRPRFFLLENVRNFVSFNKGQTFRLTLASLLEMGYQVRFGILEAGAYGVAQSRKRAFIWAAAPGETLPEWPEPMHVFASPELKITLPDGKFYAAVKSTAAGAPFRSITVRDTIGDLPAVENGAGKPTIQYGSGPVSWFQKKIRSDMASLNDHISKEMNELNLIRCKHIPKRPGCDWHDLPDEKVKLSTGQMVDLIPWCLPNTAKRHNQWKGLYGRLDWEGNFPTSVTDPQPMGKVGMCFHPEQDRIITVRECARSQGFPDSYRFAGNIQNKHRQIGNAVPPPLAYALGRKLKQAIDAKR

SEQ ID NO: 47 - DNA, 13449 bp, Oryza sativa
  misc_feature (0)...(0): AF462029.1; GI18653390 (japonica cultivar-group) putative cytosine-5 DNA methyltransferase gene, complete cds
tcgaattggagctcattaggaagttaggcaaccaaatatatagataatctagtattctgtattcggttggtccttcttatttctaagttattctggaataagagaggagaaaaatcctaatgtgggcacactgattcattccatattttttatgactcttgcaacgtattcataaagcaatagtattggagtaacaactctacccagtgtccaaccaaaattttgtaaatttggggagtcttactgccaacagctgccaatagaaacacttgatctgttaccagttaattcttgtaatatcgtgcttcaaccattgatttatacctttaaccagtctgcagaaagtgtatacatgaggagttcgtcgataaggagtcctgctgctgccctacttgcaacattgatctggggtgtgctccgttggagaaactcaggtttgtctttgtcactgtttccctgaaatattttatctacactattgcatacttctgaggcttaaaaccattgttcagttattgtattattttcttttctatgtcatgcccccaatttgatattgcgatttcaatgcagcgcatgtacatacacctaagaagttagtgacatttgggagttgggagagttttttctttaccacctctgctctcttgcatgtacaaatgagatagtcttctgtgagcaatcatccttcctccatcaatataacaactattgtgtcctgcttttttgatattattatgcatacaacactatgttcttcctcaattgccagtgataatcgtgattttgcaataccagcctagtgctgtgccactgttctgtttgcatcacatggactgttataacatcataataatctccatctgttcaatcttggcttggatggtcttaaagactttataattgttttgtttttgctcggtttattttaatgattagttgtttgttcgttacaagttataacttcgaaaggggtgagatgagcccaaacttagttcacacgatcacaccttgattaatctttagccgcttgataccgagttatcagcagcatataacaaacaagttttactttccctttttgtgatattgtagcttatggtattttcaattgtgttttgttcagagttgatcacagtatgcaattcgtaaggtcaaggatatttcctttcaaacgaaggaaggttgagaacccagaaatcatttgtccagttgcgtcacctgtcaagaggaaagaaagatcactatcttcacttacaatccctgctcctcaggtgtctatacagaaatgtttgacaaagaggagaacgaaagcttcgtgcttacgcaactttcctttggtatgtagttctcttgtaatagttcccctgagaatcggatgtgtgaagaattctaatttcttatctgtaaatccactattagaattgttcaaatgaatgtgactctgcatcaccagaatgctgtaagcaaccctataggttaggtcataccatgtatttccattgttttagtgccatagagccatctacatggtatgccagctattgtcgtattattgcagctggcacaatcacagatgtgtgctaggtaacagttcttggcactgtccgcatgtgttattggtatgctaaaggttgtgatagtcaacatgtgaatatttacgggctaatacataacaaatggttatctgaatcttctgaatttatttatagtccaggcgacattaatgatagatgcacaatacttttattttgccttcaaaactcaaatagtttcctacaaattgaggcaatccatagaaatttcatactccaaatacctttgttccaaagacatataggaaagtttcgttaggattgaaattcttcaaaaatcctccaaataccttttgttgcaaaggagatgttatgcatgaattaaatgcaaccgatgcccctcccctccaaattctaaatcgatgcgatttttattgctgttgtgtagagtatttttttctggtctgattttaatgaataaattttcttttccccgccttgtgatgtagcattctacctcgaggggcagtaaagatacatcaaagaaacttgggggttggagaccattgggttgtcaacttaagcttggcaaagacaaaaaatctctcaaatcaagtgtaaaagatacaaacagaaccaaaagtaagtctggtgatacagatgatggtgctcctgctagtaaagcaaaggctagagaaccctttacaagatatgggcgtgcagctaagaggactggaagaaagaaattgctcatgttgaagaacaaaaagaaaaggttcaaggcaaagcagcccagtaaaaagaggagattccgagcactatggttttatctacttgctgcttttgaccagtgagaaacactagacttgtcttgtgagatttgtgccgtttcttcaaaggaaaagcagaaatcatgctttgtgtttgtaattatttcaggagaggagtaccaactttaccacaactaccagcaaagtatttgaggatcaagtgagttatttggttgcacttttctagtcaaaacagtatttgttaagccacactaacaactgccttttgttatatttgtcaaagggatgttgatttgcctgcttctattatacagaagtaccttgcacagaaacttaacctctcaagtgaaactgaggtatgtctttacatatttctttcttgtagtgatcatcaaaaccagtctgttttttgtgccagtatccagattagttagaatcagcagtgagaaagaattccatcaatatttggctttcctgccagggacaagactggctaactggagctaaaatctcacaatctaaacatagttagatgtagttatggcacattcttagctgttctttggaggaaaacaacttacttcatgattatatttgaaaaggcaagaaaattcaccatactgtttccaattggtgcttgattgaaggctgaagctgaccggtgttcagttgattttgtgctgtagatacttttgccactttgttttgcgcataaccacgtttgtctttgcaatgaacaacataactgttcgctgcttcaagtttcatggtgctgcttgacacttgcaacatgtgaaattattaccctatattgacaaaagttgacagaacattctgctcattgcacagcctattttcttctgctctcaccaacgagtcgtggttgcaggtagaagtgttgtgtggtggcaaagtagtgaaccaagggatgacactgcatgatctagcagattgctggcttgagaaaggaccaaagagccgaatgcgctcatcggtaggctccccggccactggattcatggtgacattgttctatagaaggccagatgtggatgtgtcctcatccccagctccaccccaacctgacactgaaagttgccatagctgatgcagagctttgcttcgtttctgaaccatctgtgattggcttcactcattgggctgtcagcccttgattgatctgcgaatcggttccaatttgtgtgaggctcaagccacaccaattcactaatatgtagataaatgctactaattttacaagccatgttgggcttctgatcatctaccttcttcaaccattattttccttttttttctcttcaaccattgtacctaggtatgtgatctgtaatgagatgttaatcgttaagtatcagttgttagagctagggacttcatgttgtctccacgtagctagtagtactagattatgcttgtgtatttgaatttgctggtgcattcatcatgcatgtatatatataagctctggaattccttgtagttaactggatgttaagctgagaatgtataagctctagattatcagtttaccactccatgcgtgatgaaacgtgctactgcttactctgttcccaaatataaataacttgaaattgtttaccagagatcaaggtttgaaaagaaaatgctacagtgtttcttattaaggccgagtttagttacaaacttttttttttcaaacttccaacttttccatcacatcaaaactttcttacacacataaactttcaacttttctttcacatcgttccaatttcaaccaaacttcgaattttagcatgaactaaacacgccctaagtggagagtggttagggctcattcgggatgtaggttgaacgaacacagtgattggaaaaaaaataggaatgtgataggaatacatgtacaaaacatatgatttgaatatacataaatttcgtaggaacagatggctaggtgaatacacagtacagggtgtgtttagttcacgccaaaattggaagtttggttgaaattgaaacgatatgatggaaaagttgaaagtttgtgtgtataggaaagttttgatgtgatggaaaagttggaagtttgaagaaaaagtttggaattaaacttggccacaatctaattaacgcaatataaattcattttgtatttcattctcttctttttctgcttattattattaccccctatgtttcaaaatgtttgacaccgttgactttttagcacgtgtttgaccattcgttttattcaaaaaatttaagtaattatttattcttttcatatcatttgattcattgttaaatatactttcatgtacacatatagttttatatattttacaaatttttttagtaagacaaacggtcaaacacgtgctaaaaagtcaacggtgtaaaacattttgaaatggagagagtattatttttaaaaggaagccaaagtccaaactcgaaatattcggggctcccgcccaaggggtcccgctctctcgtctcctcgggactcagccccaaaaactcaaatcccccgccttcttgtcccctccgcttccccttccacttccacccccacgtcgcctcacctcgcctcctctccccctccaaaccccaccaccacagagaaaaccccagggagaaggacaagggctccacataccaacggcgccctcctcctcgactagctcccgccggtaatcccctctccccctcctcgcgctgcttcgattgcttggttcgcgtggcgcgattgcgcgtgcggtggtgggttttggttggtagttttgtctctgccttggttgcttgtgggggttcgtcgctgatcgtggtgtcgtggggagagctgatcgcggtcgcgtgatgcggtgtgtctgcggcgtctcggtcggctcgcgtccggcttcacgctgtgttgttttctgacgcgatcgtacattcgccgagattttttttttgggtgtatcggcgtggtgggtgaggcggcgattttgttcgtctgctccgtatcatcttcgatcgcttgttccactgctgatgctgtgcgcgagcgtgtcctctatttcgtctgtgcgaatgtggatgagtccatttgagtttttggtgccatttttttcatgctccgagtagcatggcgctttgatggttaatgcggctggttttgttgtgcaggggttgcgaactcggcgatggatcagtgacccgccgtggtgaagcctgcagattctacctataaggtatatcgccccacccctcttcctcggatttactagtagctgaattgttgttgactggtgaatgataatcgagcggaagctgttcaggattttgcactgctgcttgttatgctctgtgtggccctctagtaatgtggctttcattaaatcagtggttgctgcaccactgttgaaaatgcattgcactatttacacattcaacaatctatgtggtataatactaatgagaataaagtgtttattacattattccattatctaaataaattatttaattttagtgcactgtagtacgttacacattcagcaatctatgtggggttgttaacttagcgcatttgtgtttgcacatgggaatgggatcaacttgtgttgtacactagtataactgtagcttccttatggagcctagctcaatatgctatagaaaccgtctgctgaaactaataggattctccaagaaggagatgatgcggtgatggtgtgttgctgttattattttctttttgtaaactgtttttgatgtcatacaaacttcttgatgtacttgctaaactcttgagttttgcattttggttcctatatttgttttgaacttctaattgaaatgcctgcttgattccaaatttacaggggagctatggcgaaaagtccacgttctgttgttaccacaggtcttcttctgtctactttgtaactgcttatctcactttcacataacatctcatgcatttatgatttaactaagttttagtcagatcgtaaggcagtttatgcagtgtagcttacagcttatttttaccattgtgagttactgaattcaactagagccacacacatatataattatatgcctgcatatatcactcataatcacttggagttatcatgtttgatcttgcttgcaatctagaatcttgcaatagctttctacatatacatgcttgacagttataagtaagatgctgatgttgatttacttgtttatatttttaacgtgcttggttcatctgatggatacatgcttgtatggtatccaattatttcaaattgtaatataaccaacaccattttgtctctcaggaacaaaaaggcgtagagcaaaggttcataaagaagatgagcctgttgagaatgaaaacttggagagtgaatttgatgtttccaagaaagagagcaatggtgccactgaacctggtaatgagcctgttgccagcaagagaccgaagagagcagctgcctgttctaacttcaaagagaagtcattggacttatcagaaaaagattcaattatcacaatcaaggaaagtcgggttgaagagaaggaaatagaggctgttaatttgacaaggacgggacctgaagatggtcaaccttgcagaaaaatcatcgatttcatcttacatgatggagatggtaatctgcaaccctttgaaatgtctgaagttgatgacattttcataacagctcttatcatgcccttggatgatgatctggaaaaggataggggaaagggaatatgttgttcggggtttggacgaattgaaaactgggcgatttctggctatgatgaaggtgctgcagtaatttgggtctcaacagaaacatcagattacaaatgtgtgaagccagcaagcagttacagatcttattttgaacactttagtgagaaggcacgtgtctgtgttgaagtctataagaagttagctagatcagttggtggaaatcctcaggtggacttagaagaattaattgctggtgttgtccgttccattaattcaaacagaagcttcaacggaacagtaaccaaagactttgtgatctcctctggtgagttcatatataaacagcttattggattagaccatacagctggcaatgatgatgagatgttggccacactgccagttcttgttgcactgaaagatgaatgtaaatcaagagcaggattcacacatttgccagctatgccctcgaatggaactctgaggattaaggatgggcaagacaagggactgactgaggatgaggatgcaaaattagcaagactgttgcaggaagaggaagaatggaaaatgatgaagcagagaggcaagcgtggaacttcacagaaaaatatctacatcaagatttgtgaaactgaaattgccaacgactacccacttccagcctactataaaccatataaccaagaaatggatgagtacatatttgatagtgatattggtatgtattctgatgatgtacctgtaagaatccttgacaactgggctctatacaattcagattccagactcatttctttggagctcatccctatgaaagctggtgcagaaaatgatattgtggtatttggatctggttttatgagagaggatgatggtagttgctgttcaacagctgagctagcacagttacattcttcctcaagtaaatctggccgggaagatccaggagttccaatttatttgagcccaattaaagagtgggttgtagaatttggtggttcaatgatctgcataaccattcgaactgacgttgcttggtaaataccctggcagttctattttctttttgtattaccattatctccaaggggtaccatattttagctttgttagtcttgatcattgccagctcatgatggaaaaataaactcaatgcatttcggataacatatcttacacacacacacacacacacacgaatttggcattttgtttgaagcatggaattttgcaaccatgttgtgtttaccttctctctaatttacatctggtaatcaattccaggtacaaattacgccagccaacaaagcaatatgctccatggtgtgagcctgtgctgaaaacagcaaggctagctgttagtatcatcacccttttaaaagagcaaagtcgcgcttcaaagctttcttttgctgaagttatcaagaaagtagcagaatttgacagtagacaccctgcatttatatcatcgaaagcaccaaccgttgaaagatatgtcgtggtgcatggacagataatacttcagcagtttgcagactttccagatgaatctgtcaaacggtgtgccttcatcacaggtcttctagcaaagatggaggaaagtaggcacacaaagttggccatcaagaaaaaatctcaacagatgagaggggagaatctgaacccaagcgcaaaaatgggtccaatactgagaaagaagcttatgcgtgctacaactacaatgttgatcagcaagatatggggtgaatactatgccacttatttccctggggatacaaaggaagaagatcagaatgaaccaaaggaaattgatgatgatcaagaagaaaatgaagacaatgatgctgaagaggaggtaaatgttcaagatgagaaggccacaaggactccaccatcaacacggtctagaaagtcgtcagcagatactcgcaaggaaatcaaatgggaaggtcaaacagctggaaaaacagtgtctggagaagttctgtacaaatgtgttattgttcaagacctcagtatttctgttggtgcgacagtcacaacagaggatgattcaggagaaaccatcatgtgttttgttgagtatatgtatgagaaacttgatggtaaaaatatgattcatgggataattctgcaagaaggttcacagactgttcttggcaatgctgcaaatgatagagaggttttcttgactaatgactgtttagaatttgaagcaagtgacatcaaagagttggtgactgttaatatccaatcactgccttggggccacaagtacagaaaagagaattctgaagctaagagaattgaaaaggccaaggcagaggagaggaaaaggaagggcctgccagtggaatatatttgcaaaagcttatactggcctgagaaaggtggattcttctcccttccgtatgataaaattggaaatggcacaggcatctgtagctcctgtgagagaaaaccagttggcaatgaattcaagttactttctgagagcagctttgtctttgagaatattacgtataacatccatgactttctgtatatcaggcctgaatttttctcccaaggggagggccatgagacctacaaggctggaaggaatgtgggtctaaaaccttatgcagtctgccatctgctgagtgttcatggtcctgctggatcaaggaaagctaatccagaatcgacaaaagtgaaagtaagaaggttttaccgacctgatgacatttcatcaacaaaagcctactcatcagacatccgagaggtttgccttttttccatcatctgcatcattggcaatactgtgatttcacctaaacctatcttttttggcctttggtatttgattgttgtgtactttgtgatttgatccaggtgtactacagtgaagatataataagtgtacctgtggtgatgatagagggaaaatgtgaggttcgactgaaggatgaccttccaaattcagatcttccagcggtggttgaacatgtcttttgttgtgaatatttatatgatcctgctaatggagctctcaaacaggtcagctactgccaaatttttcttcagaatccctagttatctgcattgtttccactgggagatgtctttgtattattgaccgagcttgtcttgcatgatctttaaccagctaccgcccaatgttagacttgtgacactgacaaggaaggtacctgcttcaaaaaagaacaaaggaaagcaaatttgtgacattgagctaggtggttcagacaaaccaaaggatgggcaatcagagaactgtcttgcaacacttgacatttttgctggttgtggaggtttatctgaaggattgcagcgatcaggtatgctttgctcatgtagatgttgcttcataggaacattttgactccagttaccttctgaccattggattgtacaggattgtcacttactaaatgggctattgaatatgaagaacctgctggggatgcatttggtgaaaaccatccagaagctgcagtatttgtcgaaaactgcaatgtgattctgaagtacgccatttttgtttaccctctttgatatgcttatcatgtatatgtaaattgtatcttcagcacgtatctctatacgatcatgcagggcaattatggacaagtgtggtgattctgatgattgcatctccacttctgaggctgctgaacgagcagctaaactttctgaggacaagattaagaatctgcccgtgcctggcgaagtagaattcataaatggtggccctccgtgtcaggtcagttgctatgtggcttttgcctgtataccagggagctcctaacaacacattcgacattgcaagccaattgcttgaccttttgacctatccttttttagggtttttctgggatgaacagattcaatcaaagtccctggagcaaagtccagtgcgagatgatcttagcattcctgtcatttgcggagtatttccgtcctagattctttctcttagaaaatgttaggaactttgtctcgttcaacaaaggacagaccttcagattgacactggcatcactcctggagatgggataccaggtgcttgacacttcctcttcacttgtgcttgtgctatagcatttccatttctgtatacattctaaccttgtttacatgttcttaggtccgatttggaattttagaggcaggggcttatggtgttgcgcagtccaggaaaagggcattcatttgggccgctgcacctggagagactcttccagagtggcctgaaccaatgcacgtctttgctagccctgagctgaaaataactctacctgatggcaagttctacgccgctgtcaagagcaccgctgcaggagcccctttccgctcaattacagttcgagatacaattggggatctaccagctgtggaaaatggcgccggcaaaccaacaattcaggtataccctacatatcgcactagcttcactcgccaagttctcctgttcttaagctgccgctttatgtcagttgaataaactttgtatgatgtgctacagtacggaagcggtcctgtgtcttggttccagaagaagattagaagcgacatggcttcactgaatgaccacatatctaaagagatgaatgagctgaacctcataagatgcaagcacattccaaagcgcccaggttgcgactggcatgacctgccagatgaaaaggtactaacatttggccctctaattaacttctcctgcctcctgttttatttttaaactctgtaaacaccaattactgttcattgactgtgcaagtacaggtgaagctgtccacagggcagatggtggacttgatcccttggtgcttgcccaacacagccaaaaggcacaatcagtggaaaggactgtacggtaggttggactgggagggcaatttccccacttctgtaacggatcctcagccaatggggaaggtcggcatgtgcttccatcctgagcaggacaggatcattactgtccgtgaatgtgctcgatcccaggtacacataccaattttcacaccccatacattcactgctgcaacaggttaatgatgcttaactaatcatcaagtcattgactaacccaaacaaacaaattttcaggaagttttatccttcaaagtaaatttagtactacattttgtctcaatcagcactgtagcagtagatttagttctttaaccataaatcaatggatatattgtcatctctcttttcggcagaactgctttgtccattccttcttgaacctgttcaaacatgcattcattctaccgagatgccattattgcatctgcaactttgttgccctttttctgaatcttctgatctgtttctgaatcttctgatctgttcctacatgacactgtcaccattgtatgcacgcagggcttccccgatagctaccgtttcgctggcaacatccagaacaagcacaggcagatcgggaatgccgtgccaccgccccttgcctatgccctcgggaggaagctcaagcaagccatcgacgccaagcgttgagtggcttttaacttcactgcatcgccctcattttttggtcggtccaaataggtttaactaagcattacagttttctatattttgtgagcaattggactcctaaaattaattctgggatggttacatggattaccttttgtatatctaacttgctggtaggactctgataccatcaagatattggttcatagaactatagaagttcagaagagaatcatagcactgggggggggggggatagaaagcttttgtaaacagtacaactcttattaatatgactgcaatatgatgaggattagcataatcagaattaattctcgttttccagagttgtgtattggcaaactggcaatatcagcttttgtgctaggcaaacatgtccctgcttcaggtcagtgccacttgataatatacagctttcttacacagctaattttttcaaaataaatccttttcttgacctgttggtttattcatatgaacattcgatgtattgcattttgatcttgatgttatgttcagttcacaacttgatttttctttctttctttttattttgagaagggaaggatggatggcttacagttaggcaggctgacaattttcctccaaagcaacttgaaatcatcataatcagcccaaaaaattcacccaaatgagcatactacatcaaacaaatgtaaaactcccttgaaaaatgaaaacgaaaattctatacacaacattgcaagctacagaaatccaagaacacaagcacaagatcagaatcacatcaagaatcctcttagaagaagaaaaaaaaacaccttcgtctcatctcatttcagtgtgttgatgcttcttcatcttg

SEQ ID NO: 48 - protein, 284 aa, Marchantia paleacea var. diptera
  PEPTIDE (0)...(0): gi | 24416628 | dbj | BAC22505.1 | cytosine methyltransferase
QRVWSKVQCEMILAFLSYADYFRPRYFLLENVRNFVSFNKGQTFRLTMASLLEMGYQVRFGVLQAGNFGVSQSRKRAFIWAAAPDESLPDWPEARHVSASSQLGVTLPGGGQYAAVRDAGLGAPFRAITVRDTIADLPPVANGADTLKTVYTQPAESWFQMHIRGKTDVLTDHISKEMNELNLIRCQRIPKRPGADCRDLPAEKIKLSTGQLVDLIPWCLPNTAARHNQWKGLFGRLDWDGNFPTSITDPQPMGKVGMCFHPVQNRIVTVRECARSQGFPDSYK

SEQ ID NO: 49 - DNA, 855 bp, Marchantia paleacea var. diptera
  misc_feature (0)...(0): AB080617.1; GI24416627; gene for cytosine methyltransferase, partial cds
tcaaagagtatggtctaaagtacaatgtgagatgattctagcgttcttatcctacgccgactatttccgtcctcgatacttcttgcttgaaaatgttcggaacttcgtgtcattcaacaagggccaaactttcagattaacaatggcctctctcctcgagatgggttatcaggtacgctttggcgtcctacaagctgggaactttggtgtttctcagtctaggaagagggcattcatctgggcagcagctccagatgagtcattaccagattggcctgaggccagacacgtctctgcaagctcacaactaggagtaactttgcctggtggtgggcagtacgccgcagtgagagacgcagggctgggtgcccctttcagggccattactgtcagagacacaatcgctgaccttcccccggtggctaacggtgctgacaccctaaagacagtctatacccaacctgctgagtcgtggtttcaaatgcatattagagggaagaccgacgtattgactgatcacatttccaaggaaatgaatgaactgaatttgattcgctgccagcgtattcccaaaaggcccggggccgattgccgggatcttcctgccgagaagattaaattgtccacaggacaactggtcgacctcataccctggtgcctgcctaatacggccgctcggcacaaccagtggaagggtctctttggacgtcttgattgggacggcaattttcccacttcgatcaccgatcctcagcccatggggaaagtaggaatgtgcttccatcccgttcaaaatcgaattgtcacagtccgagagtgtgcccgctctcaggggtttccggattcctataagtt

SEQ ID NO: 50 - protein, 372 aa, Artificial Sequence
  consensus sequence
MEKXGDXDDCXSTXEAXELAAKLXEXXXSXLPXPGQVDFINGGPPCQGFSGMNRFNQSSWSKVQCEMILAFLSFADYFRPRYFLLENVRXFVSFNKGQTFXLTLASLLEMGYQVRFGILEAGAYGVSQSRKRAFIWAAXPEXVLPEWPEPMHVFXXPXLKIXLSXXXXYAAVRSTAXGAPXRXITVXDTIGDLPXVXNGXSXXNXEYXXXXVSWFQKXIRGXXXXLTDHIXKXMNELNLIRCXXIPXRPGXDWXDLPXXKVXLXXGXXXXXIPXCLPNTAXRHNXWKGLYGRLDWXGNFPTSVTDPQPMGKVGMCFHPXQXRIXTVRECARSQGFPDSYXFXGXIXXKHRQIGNAVPPPLAFALGRKLXXAX

(19) United States
(12) Patent Application Publication
(10) Pub. No.: US 2005/0081261 A1
(43) Pub. Date: Apr. 14, 2005

(54) Methods and compositions for altering seed phenotypes

(76) Inventors: Roger I. Pennell, Malibu, CA (US); Van-Dinh Dang, Oak Park, CA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/966,482

(22) Filed: Oct. 14, 2004

Related U.S. Application Data
(60) Provisional application No. 60/510,924, filed on Oct. 14, 2003.

(51) Int. Cl. A01H 1/00; C12N 15/82

(52) U.S. Cl. 800/278

(57) ABSTRACT

Plants are disclosed that express a cytosine DNA methyltransferase and that can be used to confer an altered seed phenotype, e.g., an increase in seed weight. Also disclosed are plants in which expression of an endogenous cytosine DNA methyltransferase is inhibited and that exhibit an altered seed phenotype, e.g., an increase in seed weight. Also disclosed are nucleic acids and polypeptides suitable for conferring such phenotypes.

### CROSS-REFERENCE TO RELATED APPLICATIONS

This application claims the benefit of and priority under 35 U.S.C. § 119(e) to U.S. Provisional Application Ser. No. 60/510,924, filed Oct. 14, 2003, which is incorporated by reference in its entirety herein.

### TECHNICAL FIELD

This invention relates to methods and materials for modulating phenotypes of plant seeds. In particular, the invention features nucleic acids and plants that can be used to modulate seed weight.

### BACKGROUND

Genes often are differentially expressed during the development of an organism and in particular cells in an organism. Elucidating and manipulating an organism's temporal and spatial gene expression profile can be useful for developing new and improved biological products.

Among the array of regulatory mechanisms that affect an organism's gene expression profile, the regulation of gene methylation has an important role. In many cases, gene methylation is regulated through site-specific methylation or demethylation of particular nucleotide sequences.

### SUMMARY

The invention involves modulating transcription and/or translation of a cytosine DNA methyltransferase-related nucleic acid in male gametophyte-specific cells or female gametophytic-specific cells in a plant. When such a plant is used as a parent in a cross, the resulting seeds have an altered seed phenotype, e.g., an increased seed weight. Thus, the invention features methods for the production of seeds. In one aspect, such methods comprise permitting a first plant to pollinate a second plant. The first plant has a first recombinant nucleic acid construct comprising a male gametophyte tissue-specific regulatory element operably linked to a first nucleic acid sequence effective for increasing levels of cytosine DNA methylation. The second plant has a second recombinant nucleic acid construct comprising a female gametophyte tissue-specific regulatory element operably linked to a second nucleic acid sequence effective for reducing levels of cytosine DNA methylation. Seeds that develop on the second plant have a mean seed weight that is increased compared to the mean seed weight of seeds that develop on a corresponding control plant that lacks the second recombinant nucleic acid construct and was pollinated by a corresponding control plant that lacks the first recombinant nucleic acid construct. Such seeds can have a mean seed weight that is at least 10% greater (e.g., 10% to about 50% greater) than the mean seed weight of seeds that develop on the control plant.

The first plant can be an inbred, a hybrid, a heterogeneous population, or a synthetic population. The first plant can be heterozygous for the recombinant nucleic acid construct or homozygous. Similarly, the second plant can be an inbred, a hybrid, a heterogeneous population, or a synthetic population, and can be homozygous for the recombinant nucleic acid construct, or heterozygous. The first and second plants can be dicotyledonous plants. The nucleic acid sequence of the first recombinant nucleic acid construct can encode a cytosine DNA methyltransferase having a region within it that has the consensus sequence set forth in SEQ ID NO: 50. The cytosine DNA methyltransferase can have 50% or greater sequence identity to one of the amino acid sequences from Arabidopsis, peach, pea, carrot, tomato, or tobacco set forth in SEQ ID NOS: 28, 30, 34, 36, 38, and 40. The second nucleic acid sequence of the second recombinant nucleic acid construct can be transcribed into an interfering RNA or an antisense nucleic acid.

The first and second plants can be monocotyledonous plants. The first nucleic acid sequence of the first recombinant nucleic acid construct can encode a cytosine DNA methyltransferase having 50% or greater sequence identity (e.g., 70%, 80, 90%, or 95%) to the amino acid sequence of either the corn or the rice cytosine DNA methyltransferase shown in SEQ ID NOS: 44 and 46.

In another aspect, the invention features a method for the production of seeds that comprises the step of permitting a first plant to pollinate a second plant. The first plant has a recombinant nucleic acid construct comprising a male gametophyte tissue-specific regulatory element operably linked to a first nucleic acid sequence effective for decreasing levels of cytosine DNA methylation. Seeds that develop on the second plant have a mean seed weight that is decreased compared to the mean seed weight of seeds that develop on a corresponding second plant pollinated by a corresponding first plant that lacks the recombinant nucleic acid construct.

In another aspect, the invention features a method for the production of seeds, that comprises the step of permitting pollination of a plant has a recombinant nucleic acid construct comprising a female gametophyte tissue-specific regulatory element operably linked to a nucleic acid sequence effective for reducing levels of cytosine DNA methylation. The pollination occurs with pollen that lacks the recombinant nucleic acid construct. Seeds that develop on the plant have a mean seed weight that is increased compared to the mean seed weight of seeds that develop on a corresponding plant that lacks the recombinant nucleic acid construct pollinated by a plant that lacks the recombinant nucleic acid construct. The pollinated plant can be a dicotyledonous plant or a monocotyldonous plant. The female gametophyte tissue-specific regulatory element can be, e.g., the Arabidopsis YP0102, YP0102a or YP0285 promoters, SEQ ID NOS: 6, 25, or 22. The nucleic acid sequence effective for reducing levels of cytosine DNA methylation can be transcribed into an interfering RNA or an antisense RNA, and can have a length of from 10 nucleotides to 4,500 nucleotides and 70% or greater sequence identity to one of the nucleic acid sequences from Arabidopsis, peach, soybean, pea, carrot, tomato, or tobacco set forth in SEQ ID NOS: 29, 31, 33, 35, 37, 39, 41, or complements of one of these sequences. Such a nucleic acid sequence can have a length of from 20 nucleotides to 1,000 nucleotides and 80% or greater sequence identity to one of these same nucleic acid sequences from Arabidopsis, peach, pea, carrot, tomato, or tobacco, or their complements. Alternatively, the nucleic acid sequence can have a length of from 10 nucleotides to 4,500 nucleotides and 70% or greater sequence identity to one of the wheat, corn, rice, or liverwort nucleic acid sequences set forth in SEQ ID NOS: 43, 45, 47, 49, or complements of one of these sequences. Such a nucleic acid sequence can have a length of from 20 nucleotides to 1,000 nucleotides and 80% or greater sequence identity to one of these same nucleic acid sequences from corn, rice, wheat, or liverwort, or their complements. The pollination can occur with pollen from a non-transgenic plant.

The invention also features a method for the production of seeds, comprising the step of permitting pollination of a plant has a recombinant nucleic acid construct comprising a female gametophyte tissue-specific regulatory element operably linked to a nucleic acid sequence effective for increasing levels of cytosine DNA methylation. The pollination occurs with pollen that lacks the recombinant nucleic acid construct. Seeds that develop on the plant have a mean seed weight that is decreased compared to the mean seed weight of seeds that develop on a corresponding plant that lacks the recombinant nucleic acid construct pollinated by a plant that lacks the recombinant nucleic acid construct.

The invention also features a method for the production of seeds, comprising the step of permitting a first plant to pollinate a second plant. The first plant has a recombinant nucleic acid construct comprising a male gametophyte tissue-specific regulatory element operably linked to a nucleic acid sequence effective for increasing levels of cytosine DNA methylation. Seeds that develop on the second plant have a mean seed weight that is increased compared to the mean seed weight of seeds that develop on a corresponding plant pollinated by a plant that lacks or does not express the recombinant nucleic acid construct. The first and second plants can be dicotyledonous plants or monocotyledonous plants. The nucleic acid sequence effective for increasing levels of cytosine DNA methylation can encode a cytosine DNA methyltransferase comprising the consensus polypeptide region described herein.

The invention also features a method for the production of seeds, comprising the step of permitting pollination among a plurality of plants that comprise a plurality of first plants. Each of the first plants has a first recombinant nucleic acid construct comprising a male gametophyte tissue-specific regulatory element operably linked to a nucleic acid sequence effective for increasing levels of cytosine DNA methylation, wherein seeds that develop on the first plants after pollination have a mean seed weight that is increased compared to the mean seed weight of seeds that develop on corresponding plants that lack the recombinant nucleic acid construct. The pollination can be predominantly self-pollination. The plurality of first plants can be dicotyledonous plants or monocotyledonous plants. The plurality of plants can further comprise a plurality of second plants. The second plants have a second recombinant nucleic acid construct comprising a female gametophyte tissue-specific regulatory element operably linked to a nucleic acid sequence effective for reducing levels of cytosine DNA methylation. Seeds that develop on the second plants after pollination have a mean seed weight that is increased compared to the mean seed weight of seeds that develop on corresponding plants that lack the recombinant nucleic acid construct. Seeds that develop on the pollinated plants have a mean seed weight that can be at least 10% greater than the mean seed weight of seeds that develop on the corresponding plants that lack the recombinant nucleic acid construct.

The invention also features a transgenic host cell comprising a recombinant nucleic acid construct comprising a nucleic acid sequence effective for reducing levels of cytosine DNA methylation. The nucleic acid sequence is operably linked to one or more regulatory elements that confer transcription in plant female gametophyte cell types. The regulatory element can comprise one of the sequences set forth in SEQ ID NOS: 6 through 27. In another aspect, a transgenic host cell can comprise a recombinant nucleic acid construct comprising a nucleic acid sequence effective for reducing levels of cytosine DNA methylation, the nucleic acid sequence operably linked to one or more regulatory elements that confer transcription in plant male gametophyte cell types.

The invention also features a transgenic plant comprising a recombinant nucleic acid construct comprising a nucleic acid sequence effective for reducing levels of cytosine DNA methylation. The nucleic acid sequence is operably linked to one or more regulatory elements that confer transcription in female gametophyte cell types. The regulatory element can comprise one of the sequences set forth in SEQ ID NOS: 6 through 27. The one or more regulatory elements can confer preferential transcription in polar cell nuclei and central cells relative to egg cells, zygotes and embryos. The plant can be a dicotyledonous plant or a monocotyledonous plant. The nucleic acid sequence effective for reducing levels of cytosine DNA methylation can be transcribed into an interfering RNA or an antisense RNA. The nucleic acid sequence can have a length of from 10 nucleotides to 4,500 nucleotides and 70% or greater sequence identity to one of the nucleic acid sequences set forth in SEQ ID NOS: 29, 31, 33, 35, 37, 39, 41, 43, 45, 47, 49, or complements of one of these sequences. For example, such a nucleic acid can have a length of from 20 nucleotides to 1,000 nucleotides and 80% or greater sequence identity to one of these nucleic acid sequences, or their complements.

The invention also features a transgenic plant comprising a recombinant nucleic acid construct comprising a nucleic acid sequence effective for reducing levels of cytosine DNA methylation, the nucleic acid sequence operably linked to one or more regulatory elements that confer transcription in male gametophyte cell types.

The invention also features an article of manufacture comprising packaging material and two or more types of seeds in the packaging material. In some embodiments, plants grown from seeds of the first type overexpress a cytosine DNA methyltransferase in male gametophyte cells. Plants grown from seeds of the second type may or may not have a recombinant nucleic acid construct that inhibits expression of a cytosine DNA methyltransferase in female gametophyte cells. In other embodiments, plants grown from seeds of the first type lack a recombinant nucleic acid that results in overexpression of a cytosine DNA methyltransferase in male gametophyte cells and plants grown from seeds of the second type have a recombinant nucleic acid construct that inhibits expression of a cytosine DNA methyltransferase in female gametophyte cells.

The details of one or more embodiments of the invention are set forth in the accompanying drawings and the description below. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features, objects, and advantages of the invention will be apparent from the description and drawings, and from the claims.

### DESCRIPTION OF DRAWINGS

FIG. 1 shows the Arabidopsis genomic DNA sequence of Met1. The underlined nucleotides represent the portion of the genomic sequence used to make the antisense nucleic acid construct of Example 1.

FIG. 2 is a diagrammatic representation of certain features in a cytosine DNA methyltransferase.

Like reference symbols in the various drawings indicate like elements.

### DETAILED DESCRIPTION

In one aspect, the invention provides methods for modulating a seed phenotype in a plant. Modulating a seed phenotype involves transcribing and/or translating a cytosine DNA methyltransferase-related nucleic acid in male gametophyte-specific cells or female gametophytic-specific cells in an organism such as Zea mays or Glycine max. Thus, in some embodiments, a cytosine DNA methyltransferase can be expressed in male gametophyte cells of a plant, and pollen from such a plant can be used to create seeds having an increased seed weight. In other embodiments, transcription or translation of an endogenous cytosine DNA methyltransferase is inhibited in male gametophyte cells of a plant, and pollen from such a plant can be used to create seeds having a decrease in seed weight.

In other embodiments, a cytosine DNA methyltransferase can be expressed in female gametophyte cells of a plant and, after pollination, can form seeds having a decreased seed weight. In other embodiments, transcription or translation of an endogenous cytosine DNA methyltransferase is inhibited in female gametophyte cells of a plant and, after pollination, can form seeds having an increased seed weight.

Modulating Seed Phenotypes Via Overexpression in Male Gametophyte Cells or Underexpression in Female Gametophyte Cells

Overexpression in Male Gametophyte Cells

In a first aspect, the invention involves permitting a first plant to pollinate a second plant and thereby produce seeds on the second plant. The first plant contains a recombinant nucleic acid construct comprising a nucleic acid encoding a cytosine DNA methyltransferase polypeptide, operably linked to one or more regulatory elements that confer expression in male gametophyte cells or tissues. By expressing a methyltransferase polypeptide in specific male gametophyte cell types, it is possible to modulate gene expression in the first plant (e.g., by inactivating genes that normally are transcriptionally active) and achieve one or more beneficial seed phenotypes when the first plant is used to pollinate a second plant.

Cytosine DNA methyltransferases suitable for use in the invention can be characterized by evaluating the phenotype of loss-of-function mutants in the gene for the methyltransferase. Such mutants exhibit global hypomethylation of cytosine residues in gametophyte tissue. Furthermore, such mutants exhibit a reduction in global cytosine methylation in both single copy and repetitive sequences in the genome, although the hypomethylation of repetitive sequences can be more modest. The existence of such mutants indicates that the wild-type counterpart is a cytosine DNA methyltransferase suitable for use in methods and compositions described herein.

A number of cytosine DNA methyltransferase polypeptides are suitable for use in the methods described herein. One such polypeptide is the polypeptide encoded by the Arabidopsis Met1 gene. The nucleotide sequence encoding the Arabidopsis Met1 DNA cytosine methyltransferase is shown in SEQ ID NO: 29. The Genbank accession number for Arabidopsis MET1 is AT5G49160. In addition, a corn cytosine DNA methyltransferase having the amino acid sequence shown in SEQ ID NO: 44, and a rice cytosine DNA methyltransferase having the amino acid sequence shown in SEQ ID NO: 46 are also useful.

Organism Table

| Organism | Organism ID |
|---|---|
| Zea mays | 311987 |
| Glycine max | 3847 |
| Triticum aestivum | 4565 |

Other suitable cytosine DNA methyltransferases polypeptides can be identified in a variety of ways. For example, candidate methyltransferases can be screened to identify polypeptides having cytosine DNA methyltransferase activity by preparing nuclear extracts from axenic seedlings and incubating solubilized proteins from the extract with a hemi-methylated (CpI)ₙ substrate and radioactively labeled S-adenosyl-methionine. See, e.g., Kakutani et al., Nucleic Acids Res. 93:12406-12411 (1995). Global cytosine methylation levels in a genome can be measured by digesting total genomic DNA with TaqI and labeling 5′ terminal cytosines in the digest with radioactivity. The labeled DNA is then digested to mononucleotides and the amount of methylated and unmethylated cytosine is estimated using thin layer chromatography. See, e.g., Kakutani, et al., Nucleic Acids Res. 93:12406-12411 (1995). The methylation of single copy and repetitive sequences can be estimated from the digestion pattern observed in Southern blots of genomic DNA digested with HpaII or MspI. See, Jeddeloh et al., Plant J. 9:579-586 (1996) and Finnegan et al., Proc. Natl. Acad. Sci. USA 93:8449-8454 (1996). Suitable cytosine DNA methyltransferases have corresponding loss-of-function mutants that exhibit global hypomethylation of cytosine residues in gametophyte tissue, a reduction in global cytosine methylation in single copy sequences in the genome, and a more modest hypomethylation of repetitive sequences.

Coimmunoprecipitation assays using antibodies against known methyltransferases also can be used to identify candidate polypeptides. Another way to identify candidate polypeptides is by functional complementation of methyltransferase mutants.

Suitable candidates for methyltransferases also can be identified by analysis of nucleotide and polypeptide sequence alignments. For example, performing a query on a database of nucleotide or polypeptide sequences can identify orthologs of cytosine DNA methyltransferases. Sequence analysis can involve BLAST or PSI-BLAST analysis of nonredundant databases using known methyltransferases amino acid sequences. Those proteins in the database that have greater than 40% sequence identity are candidates for further evaluation for suitability as a methyltransferase. If desired, manual inspection of such candidates can be carried out in order to narrow the number of candidates to be further evaluated. Manual inspection can be performed by selecting those candidates that appear to have domains suspected of being present in methyltransferases. Suitable candidates include SEQ ID NOS: 42 and 48.

A percent identity for any subject nucleic acid or amino acid sequence (e.g., an Arabidopsis cytosine DNA methyltransferase, or a Zea mays cytosine DNA methyltransferase) relative to another “target” nucleic acid or amino acid sequence can be determined as follows. First, a target nucleic acid or amino acid sequence can be compared and aligned to a subject nucleic acid or amino acid sequence, using the BLAST 2 Sequences (B12seq) program from the stand-alone version of BLASTZ containing BLASTN and BLASTP (e.g., version 2.0.14). The stand-alone version of BLASTZ can be obtained at <www.fr.com/blast> or www.ncbi.nlm.nih.gov>. Instructions explaining how to use BLASTZ, and specifically the B12seq program, can be found in the ‘readme’ file accompanying BLASTZ. The programs also are described in detail by Karlin et al, 1990, Proc. Natl. Acad. Sci. 87:2264; Karlin et al, 1990, Proc. Natl. Acad. Sci. 90:5873; and Altschul et al, 1997, Nucl. Acids Res. 25:3389.

B12seq performs a comparison between the subject sequence and a target sequence using either the BLASTN (used to compare nucleic acid sequences) or BLASTP (used to compare amino acid sequences) algorithm. Typically, the default parameters of a BLOSUM62 scoring matrix, gap existence cost of 11 and extension cost of 1, a word size of 3, an expect value of 10, a per residue cost of 1 and a lambda ratio of 0.85 are used when performing amino acid sequence alignments. The output file contains aligned regions of homology between the target sequence and the subject sequence. Once aligned, a length is determined by counting the number of consecutive nucleotides or amino acid residues (i.e., excluding gaps) from the target sequence that align with sequence from the subject sequence starting with any matched position and ending with any other matched position. A matched position is any position where an identical nucleotide or amino acid residue is present in both the target and subject sequence. Gaps of one or more residues can be inserted into a target or subject sequence to maximize sequence alignments between structurally conserved domains (e.g., α-helices, α-sheets, and loops).

The percent identity over a particular length is determined by counting the number of matched positions over that particular length, dividing that number by the length and multiplying the resulting value by 100. For example, if (i) a 500 amino acid target sequence is compared to a subject amino acid sequence, (ii) the B12seq program presents 200 amino acids from the target sequence aligned with a region of the subject sequence where the first and last amino acids of that 200 amino acid region are matches, and (iii) the number of matches over those 200 aligned amino acids is 180, then the 500 amino acid target sequence contains a length of 200 and a sequence identity over that length of 90% (i.e., 180÷200×100=90). In some embodiments, the amino acid sequence of a suitable cytosine DNA methyltransferase has greater than 40% sequence identity (e.g., >80%, >70%, >60%, >50% or >40%) to the amino acid sequence of Arabidopsis Met1 cytosine DNA methyltransferase. In other embodiments, the amino acid sequence of a suitable cytosine DNA methyltransferase has greater than 40% sequence identity (e.g., >80%, >70%, >60%, >50% or >40%) to the amino acid sequence of the corn cytosine DNA methyltransferase shown in SEQ ID NO: 44 or the rice cytosine DNA methyltransferase shown in SEQ ID NO: 46. In yet other embodiments, the amino acid sequence of a suitable cytosine DNA methyltransferase polypeptide has a total length of from 1500 to 1600 amino acids (e.g., from 1520 to 1565, from 1522 to 1564, 1522, 1525, 1534, 1545, 1554, 1559, 1564, or 1566; a region of the polypeptide is from 350 to 390 amino acids in length (e.g., 350 to 375, 350 to 380, 360 to 380, 370 to 375, or 365 to 375, or 372) and has greater than 40% sequence identity (e.g., >80%, >70%, >60%, >50% or >40%) to the amino acid sequence set forth in SEQ ID NO: 50.

It will be appreciated that a nucleic acid or amino acid target sequence that aligns with a subject sequence can result in many different lengths with each length having its own percent identity. It will also be appreciated that the length of a suitable nucleic acid can depend upon the intended use, e.g., as a full-length coding sequence, as an antisense sequence, or an RNAi sequence. It is noted that the percent identity value can be rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

The identification of conserved regions in a template, or subject, polypeptide can facilitate homologous polypeptide sequence analysis. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at http://www.sanger.ac.uk/Pfam/ and http://genome.wustl.edu/Pfam/. A description of the information included at the Pfam database is described in Sonnhammer et al, 1998, Nucl. Acids Res. 26: 320-322; Sonnhammer et al, 1997, Proteins 28:405-420; and Bateman et al, 1999, Nucl. Acids Res. 27:260-262. From the Pfam database, consensus sequences of protein motifs and domains can be aligned with the template polypeptide sequence to determine conserved region(s).

Conserved regions also can be determined by aligning sequences of the same or related polypeptides from closely related plant species. Closely related plant species preferably are from the same family. Alternatively, alignments are performed using sequences from plant species that are all monocots or are all dicots. In some embodiments, alignment of sequences from two different plant species is adequate. For example, sequences from canola and Arabidopsis can be used to identify one or more conserved regions.

Typically, polypeptides that exhibit at least about 35% amino acid sequence identity are useful to identify conserved regions. Conserved regions of related proteins sometimes exhibit at least 40% amino acid sequence identity (e.g., at least 50%, at least 60%; or at least 70%, at least 80%, or at least 90% amino acid sequence identity). In some embodiments, a conserved region of target and template polypeptides exhibit at least 92, 94, 96, 98, or 99% amino acid sequence identity. Amino acid sequence identity can be deduced from amino acid or nucleotide sequence.

One of skill will recognize that individual substitutions, deletions or additions to a polypeptide that alter, add or delete a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
(see, e.g., Creighton, Proteins (1984)).

A consensus sequence for a region of a suitable cytosine methyltransferase is shown in the Sequence Listing. Certain symbols are used in the consensus sequence to represent suitable substitutions at certain amino acid residues and to represent acceptable length variations at certain positions:

| | |
|---|---|
| + = | “positive” e.g. H, K, R |
| a = | “Aliphatic” e.g. I, L, V, M |
| t = | “Tiny” e.g. T, G, A |
| r = | “Aromatic” e.g. F, Y, W |
| n = | “Negative” e.g. E, D |
| p = | “Polar” e.g. N, Q |
| <#—#> = | specified # of amino acids, any type |
| (X, Y) = | one amino acid residue, either X or Y |

In some instances, suitable methyltransferases can be synthesized on the basis of consensus functional domains and/or conserved regions in polypeptides that are homologous methyltransferases. Consensus domains and conserved regions can be identified by homologous polypeptide sequence analysis as described above. The suitability of such synthetic polypeptides for use as a cytosine DNA methyltransferase can be evaluated based on their effect on genome methylation status, or by functional complementation of the corn, rice, or Arabidopsis cytosine DNA methyltransferases shown in the Sequence Listing.

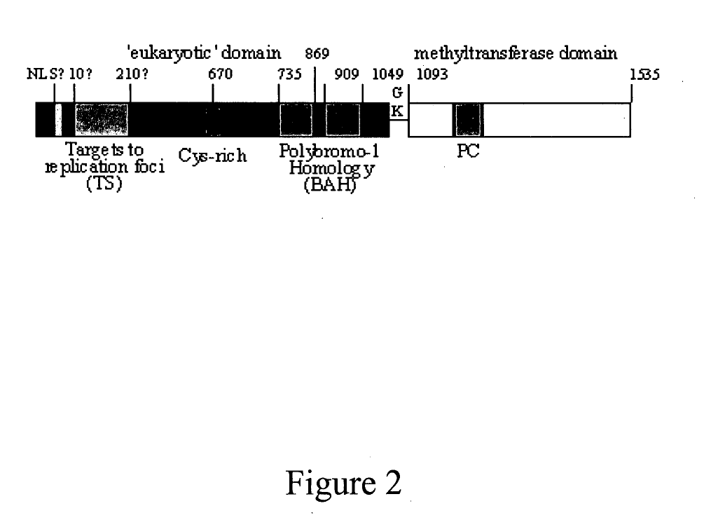

Domains are groups of contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, each domain has been associated with either a conserved primary sequence or a sequence motif. Generally these conserved primary sequence motifs have been correlated with specific in vitro and/or in vivo activities. A domain can be any length, including the entirety of the polynucleotide to be transcribed. Examples of domains that can be used to identify orthologous cytosine DNA methyltransferases include, without limitation, a methyltransferase activity domain, a “eukaryotic” domain, a TS domain, a BAH domain, a Cys-rich domain, a GK repeat domain, and a PC repeat domain. See, FIG. 2.

The recombinant nucleic acid construct in the first plant contains one or more regulatory elements operably linked to the sequence encoding a cytosine DNA methyltransferase. Regulatory elements can include promoter sequences, enhancer sequences, response elements, protein recognition sites, inducible elements that modulate expression of a nucleic acid sequence, promoter control elements, protein binding sequences, 5′ and 3′ UTRs, transcriptional start sites, termination sequences, polyadenylation sequences, introns and certain sequences within amino acid coding sequences such as secretory signals, and protease cleavage sites. As used herein, “operably linked” refers to positioning of a regulatory element in a construct relative to a nucleic acid in such a way as to permit or facilitate transcription and/or translation of the nucleic acid. The choice of element(s) to be included depends upon several factors, including, but not limited to, replication efficiency, selectability, inducibility, desired expression level, and cell or tissue specificity.

Typically, a promoter is located 5′ to the sequence to be transcribed, and proximal to the transcriptional start site of the sequence. Promoters are upstream of the first exon of a coding sequence and upstream of the first of multiple transcription start sites. In some embodiments, a promoter is positioned about 3,000 nucleotides upstream of the ATG of the first exon of a coding sequence. In other embodiments, a promoter is positioned about 2,000 nucleotides upstream of the first of multiple transcription start sites. The promoters of the invention comprise at least a core promoter as defined below. Additionally, the promoter may also include at least one control element such as an upstream element. Such elements include UTRs and optionally, other DNA sequences that affect transcription of a polynucleotide such as a synthetic upstream element.

An 5′ untranslated region (UTR) is transcribed, but is not translated, and lies between the start site of the transcript and the translation initiation codon and includes the +1 nucleotide. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences.

In these embodiments, regulatory elements that preferentially drive transcription in male gametophyte cells can be used, e.g., microspore mother cells, or microspores, including vegetative cell and the cell within the vegetative cell that divides and gives rise to the sperm cells. However, it is preferred that no transcription be observed in mature pollen nuclei. Furthermore, transcription in embryo or endosperm from the regulatory element after fertilization is not desirable. Thus, rapidly diminishing transcription in endosperm tissue after fertilization is preferred. A suitable male reproductive tissue-specific promoter is the Arabidopsis YP0180 promoter (SEQ ID NO: 8).

A cell type or tissue-specific promoter is sometimes observed to drive expression of operably linked sequences in tissues other than the target tissue. Thus, as used herein a cell type or tissue-specific promoter is one that drives expression preferentially in the target tissue, but can also lead to some expression in other cell types or tissues as well. Methods for identifying and characterizing regulatory elements in plant genomic DNA include, for example, those described in the following references: Jordano, et al., Plant Cell, 1:855-866 (1989); Bustos, et al., Plant Cell, 1:839-854 (1989); Green, et al., EMBO J., 7:4035-4044 (1988); Meier, et al., Plant Cell, 3:309-316 (1991); and Zhang, et al., Plant Physio., 110:1069-1079 (1996).

Underexpression in Female Gametophyte Cells

In another aspect, the invention provides methods for modulating a seed phenotype in a plant by decreasing the degree of genomic cytosine methylation during female gametogenesis. In this aspect, a plant used as the female in a cross contains a nucleic acid construct comprising a female gametophyte tissue-specific regulatory element operably linked to a nucleic acid sequence effective for reducing levels of global cytosine DNA methylation. The plant is pollinated with pollen that lacks the nucleic acid sequence, and seeds that develop on the plant have an average seed weight that is increased compared to the average seed weight of seeds that develop on a corresponding plant that lacks the nucleic acid sequence.

In this aspect, the recombinant nucleic acid construct can incorporate sequences which inhibit or prevent transcription and/or translation of an endogenous cytosine DNA methyltransferase. For instance, one can use antisense sequences. Suitable antisense sequences include an antisense nucleic acid that covers the portion of the gene encoding amino acids 764 to 1535 of Arabidopsis Met1, or the portion of the gene encoding amino acids 644 to 1535, or the portion of the gene encoding amino acids 485 to 1535. Such antisense nucleic acids are about 2.3 kb, 2.7 kb, and 3.2 kb respectively.

In addition, a construct that contains a whole or partial copy of an endogenous gene in sense can result in suppression of expression of the endogenous gene. Thus, the construct can incorporate additional copies, or partial copies, of genes encoding methyltransferases already present in the plant, i.e., a DNA having a sequence that is similar or identical to the sense coding sequence of an endogenous cytosine DNA methyltransferase, but that is transcribed into a mRNA that is unpolyadenylated, lacks a 5′ cap structure, or contains an unsplicable intron. In another alternative, the construct can incorporate a sequence encoding a ribozyme.

In another alternative, the construct can include a sequence that is transcribed into an interfering RNA. See, e.g., U.S. Pat. No. 6,753,139; US Patent Publication 20040053876; and US Patent Publication 20030175783. Such an RNA can be one that anneals to another RNA to form an interfering RNA. Such an RNA can also be one that can anneal to itself, e.g., a double stranded RNA having a stem-loop structure. One strand of the stem portion of a double stranded RNA comprises a sequence that is similar or identical to the sense coding sequence of an endogenous cytosine DNA methyltransferase, and that is from about 10 nucleotides to about 4,500 nucleotides in length. In some embodiments, the stem portion is similar or identical to UTR sequences 5′ of the coding sequence. In some embodiments, the stem portion is similar or identical to UTR sequences 3′ of the coding sequence. The length of the sequence that is similar or identical to the sense coding sequence, the 5′ UTR, or the 3′ UTR can be from 10 nucleotides to 500 nucleotides, from 15 nucleotides to 300 nucleotides, from 20 nucleotides to 100 nucleotides, or from 25 nucleotides to 100 nucleotides. In some embodiments the length of the sequence that is similar or identical to the sense coding sequence, the 5′ UTR, or the 3′ UTR can be from 25 nucleotides to 500 nucleotides, from 25 nucleotides to 300 nucleotides, from 25 nucleotides to 1,000 nucleotides, from 100 nucleotides to 2,000 nucleotides, from 300 nucleotides to 2,500 nucleotides, from 200 nucleotides to 500 nucleotides, from 1,000 nucleotides to 3,000 nucleotides, or from 200 nucleotides to 1,000 nucleotides. The other strand of the stem portion of a double stranded RNA comprises an antisense sequence of an endogenous cytosine DNA methyltransferase, and can have a length that is shorter, the same as, or longer than the corresponding length of the complementary strand of the stem portion. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. See, e.g., WO 99/53050.

To achieve female gametophyte specific expression, regulatory elements that preferentially drive transcription in female gametophytic tissues are used, such as embryo sac promoters. Most suitable are regulatory elements that preferentially drive transcription in polar nuclei or the central cell, or in precursors to polar nuclei, but not in egg cells or precursors to egg cells. A regulatory element whose pattern of transcription extends from polar nuclei into early endosperm development is also acceptable, although rapidly diminishing transcription in endosperm tissue after fertilization is most preferred. Expression in the zygote or developing embryo is not preferred.

Female reproductive tissue promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038); Arabidopsis viviparous-1 (see, Genbank No. U93215); Arabidopsis atmycl (see, Urao (1996) Plant Mol. Biol., 32:571-57; Conceicao (1994) Plant, 5:493-505).

Other female gametophyte tissue promoters include those derived from the following genes: Arabidopsis Fie (GenBank No. AF129516); Arabidopsis Mea; and Arabidopsis Fis2 (GenBank No. AF096096); ovule BEL1 (Reiser (1995) Cell, 83:735-742; Ray (1994) Proc. Natl. Acad. Sci. USA, 91:5761-5765; GenBank No. U39944); and Arabidopsis DMC1 (see, GenBank No. U76670).

Exemplary female gametophyte tissue-specific promoters include the following Arabidopsis promoters: YP0039 (SEQ ID NO: 10), YP0101 (SEQ ID NO: 11), YP0102 (SEQ ID NO: 6), YP0110 (SEQ ID NO: 9), YP0117 (SEQ ID NO: 7), YP0119 (SEQ ID NO: 12), YP0137 (SEQ ID NO: 13), DME PROMOTER (SEQ ID NO: 15), YP0285 (SEQ ID NO: 22) and YP0212 (SEQ ID NO: 14).

Promoters that may be useful in monocotyledonous plants such as rice include the following promoters: Y678g10p3 (SEQ ID NO: 20), p756a09p3 (SEQ ID NO: 21), Y790g04p3 (SEQ ID NO: 23), p780a10p3 (SEQ ID NO: 24), Y730e07p3 (SEQ ID NO: 26), Y760g09p3 (SEQ ID NO: 27), p530c10p3 (SEQ ID NO: 19), p524d05p3, (SEQ ID NO: 18) p523d11p3 (SEQ ID NO: 17) and p472e10p3 (SEQ ID NO: 16).

Seed Phenotypes

An organism exhibiting modulated gene expression as described above can be used to produce seeds after pollination. Such seeds can have phenotypic alterations relative to organisms that lack or do not express the methyltransferase polypeptide. For example, such modulated gene expression can alter one or more of the following seed phenotypes: seed yield, seed composition, endosperm development, embryo development, cotyledon development, seed size, seed development time, seedling growth rate, or seed fertility. Phenotypes such as seed yield, seed composition, seed size and seed weight typically are measured on mature seeds on a dry weight basis.

Expression of a cytosine DNA methyltransferase polypeptide in male gametophyte cell types can result in an increase in average seed weight of about 10% to about 50%, e.g., about 10% to about 40%, or about 10% to about 30%, or about 10% or about 20%, or about 15% to about 30%, or about 15% to about 25%, when pollen from plants exhibiting such expression are used as pollinators in a cross. Similarly, an increase in average seed weight of about the same magnitude is observed when expression of an endogenous cytosine DNA methyltransferase polypeptide is inhibited in female gametophyte cell types and such a plant is used as the female in a cross.

Typically, a difference in a phenotype such as seed weight in a plant relative to a corresponding control plant is considered statistically significant at p≦0.05 with an appropriate parametric or non-parametric statistic, e.g., Chi-square test, Student's t-test, Mann-Whitney test, or F-test. In some embodiments, a difference is statistically significant at p<0.01, p<0.005, or p<0.001. A statistically significant difference in, for example, seed weight of seeds from a transgenic test plant compared to the seed weight of seeds from a non-transgenic control plant indicates that the recombinant nucleic acid present in the test plant alters seed weight.

It will be appreciated that both parents in a cross can have modulated expression of a cytosine DNA methyltransferase, and thereby achieve even greater alterations of a seed phenotype compared to crosses in which only one parent plant has modulated methyltransferase expression. Thus, a first, pollinator plant can exhibit overexpression of a cytosine DNA methyltransferase in male gametophyte cells. A second, seed-bearing plant can have transcription or translation of an endogenous cytosine DNA methyltransferase inhibited in female gametophyte cells. After pollination by the first plant, seeds that form on the second plant have an increased seed weight compared to corresponding first and second plants that do not exhibit overexpression or inhibition, respectively, of a cytosine DNA methyltransferase. An example of such seeds is the progeny of a cross of a female corn plant containing a recombinant nucleic acid construct comprising a YP0102a promoter operably linked to a cytosine DNA methyltransferase sequence that decreases the amount of methyltransferase activity via an RNAi mechanism, with a male corn plant containing a recombinant nucleic acid construct comprising a male gametophyte promoter operably linked to a full-length cytosine DNA methyltransferase coding sequence that results in overexpression of the methyltransferase.

Modulating Seed Phenotypes Via Underexpression in Male Gametophyte Cells or Overexpression in Female Gametophyte Cells

Underexpression in Male Gametophyte Cells

In another aspect, the invention provides methods for producing plant seeds that have one or more altered seed phenotypes. The method comprises the step of permitting a first plant to pollinate a second plant. The first plant contains a recombinant nucleic acid construct comprising one or male gametophyte tissue-specific regulatory elements operably linked to a nucleic acid sequence effective for decreasing levels of cytosine DNA methylation. Upon pollination, seeds develop on the second plant have a mean seed weight that is decreased compared to the mean seed weight of seeds that develop on a corresponding plant pollinated by a plant that lacks the nucleic acid sequence. Suitable male gametophyte cell-specific regulatory elements are described herein. Nucleic acids effective for decreasing levels of cytosine DNA methylation are also described herein and include antisense sequences, interfering RNA sequences, and ribozyme sequences.

Overexpression in Female Gametophyte Cells

In another aspect, the method for producing seeds can involve permitting pollination of a plant that contains a recombinant nucleic acid construct comprising a female gametophyte tissue-specific regulatory element operably linked to a nucleic acid sequence effective for increasing levels of cytosine DNA methylation. The pollen used for pollination lacks such a nucleic acid sequence. Seeds that develop on such a plant have a mean seed weight that is decreased compared to the mean seed weight of seeds that develop on a corresponding plant that lacks or does not express the nucleic acid sequence. Suitable female gametophyte cell-specific regulatory elements are described herein. Nucleic acids effective for increasing levels of cytosine DNA methylation are also described herein and include coding sequences for cytosine DNA methyltransferases described herein.

Seed Phenotypes

An organism exhibiting modulated gene expression as described above can be used to produce seeds after pollination. Such seeds can have phenotypic alterations relative to organisms that lack or do not express the methyltransferase polypeptide. For example, such modulated gene expression can alter one or more of the following seed phenotypes: seed yield, seed composition, endosperm development, embryo development, cotyledon development, seed size, seed development time, or seed fertility. Phenotypes such as seed yield, seed composition, seed size and seed weight typically are measured on mature seeds on a dry weight basis.

Inhibition of expression of an endogenous cytosine DNA methyltransferase polypeptide in male gametophyte cell types can result in a decrease in average seed weight of about 10% to about 50%, e.g., about 10% to about 40%, or about 10% to about 30%, or about 10% or about 20%, or about 15% to about 30%, or about 15% to about 25%, when pollen from plants exhibiting such expression are used as pollinators in a cross. Similarly, a decrease in average seed weight of about the same magnitude is observed when a cytosine DNA methyltransferase polypeptide is expressed in female gametophyte cell types and such a plant is used as the female in a cross.

Typically, a difference in a phenotype such as seed weight in a plant relative to a corresponding control plant is considered statistically significant at p≦0.05 with an appropriate parametric or non-parametric statistic, e.g., Chi-square test, Student's t-test, Mann-Whitney test, or F-test. In some embodiments, a difference is statistically significant at p<0.01, p<0.005, or p<0.001. A statistically significant difference in, for example, seed weight of seeds of a transgenic test plant compared to the seed weight of seeds of a non-transgenic control plant indicates that the recombinant nucleic acid present in the test plant alters seed weight.

It will be appreciated that both parents in a cross can have modulated expression of a cytosine DNA methyltransferase, and thereby achieve even greater alterations of a seed phenotype compared to crosses in which only one parent plant has modulated methyltransferase expression. Thus, a first, pollinator plant can inhibit transcription or translation of an endogenous cytosine DNA methyltransferase in male gametophyte cells. A second, seed-bearing plant can express a cytosine DNA methyltransferase in female gametophyte cells. After pollination by the first plant, seeds that form on the second plant have decreased seed weight compared to corresponding first and second plants that do not exhibit inhibition or overexpression, respectively, of a cytosine DNA methyltransferase.

Nucleic Acids Encoding a Methyltransferase

The present invention also includes nucleic acids encoding cytosine DNA methyltransferase polypeptides, nucleic acids having homology to a cytosine DNA methyltransferase, e.g., antisense sequences for a cytosine DNA methyltransferase, ribozyme sequences for a cytosine DNA methyltransferase, or interfering RNA sequences for a cytosine DNA methyltransferase. As used herein, nucleic acid refers to RNA or DNA, including cDNA, synthetic DNA or genomic DNA. The nucleic acids can be single- or double-stranded, and if single-stranded, can be either the coding or non-coding strand. As used herein with respect to nucleic acids, “isolated” refers to (i) a naturally-occurring nucleic acid encoding part or all of a polypeptide of the invention, but free of sequences, i.e., coding sequences, that normally flank one or both sides of the nucleic acid encoding polypeptide in a genome; (ii) a nucleic acid incorporated into a vector or into the genomic DNA of an organism such that the resulting molecule is not identical to any naturally-occurring vector or genomic DNA; or (iii) a cDNA, a genomic nucleic acid fragment, a fragment produced by polymerase chain reaction (PCR) or a restriction fragment. Specifically excluded from this definition are nucleic acids present in mixtures of nucleic acid molecules or cells.

Examples of suitable nucleic acids include nucleic acids encoding the Arabidopsis thaliana, Oryza sativa, and Zea mays cytosine-5 DNA methyltransferases shown in the Sequence Listing. Exemplary nucleic acids are described at Genbank Accession Nos. AF063403 and AC093713. It should be appreciated, however, that nucleic acids having a nucleotide sequence other than the specific nucleotide sequences disclosed herein still can encode a polypeptide having the exemplified amino acid sequence. The degeneracy of the genetic code is well known to the art; i.e., for many amino acids, there is more than one nucleotide triplet that serves as the codon for the amino acid.

Recombinant nucleic acid constructs can contain cloning vector sequences in addition to other sequences described herein. Suitable cloning vector sequences are commercially available and are used routinely by those of ordinary skill. Nucleic acid constructs of the invention also can contain sequences encoding other polypeptides. Such polypeptides may, for example, facilitate the introduction or maintenance of the nucleic acid construct into a host organism. Other polypeptides also can affect the expression, activity, or biochemical or physiological effect of the encoded methyltransferase. Alternatively, other polypeptide coding sequences can be provided on separate nucleic acid constructs.

A nucleic acid encoding a cytosine DNA methyltransferase can be obtained by, for example, DNA synthesis or the polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach, C. & Dveksler, G., Eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies are available by which site-specific nucleotide sequence modifications can be introduced into a template nucleic acid.

Nucleic acids can be detected by methods such as ethidium bromide staining of agarose gels, Southern or Northern blot hybridization, PCR or in situ hybridizations. Hybridization typically involves Southern or Northern blotting (see, for example, sections 9.37-9.52 of Sambrook et al., 1989, “Molecular Cloning, A Laboratory Manual”, 2^{nd} Edition, Cold Spring Harbor Press, Plainview; N.Y.). Probes should hybridize under high stringency conditions to a nucleic acid or the complement thereof. High stringency conditions can include the use of low ionic strength and high temperature washes, for example 0.015 M NaCl/0.0015 M sodium citrate (0.1×SSC), 0.1% sodium dodecyl sulfate (SDS) at 65° C. In addition, denaturing agents, such as formamide, can be employed during high stringency hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Eukaryotic Organisms

The term “host” or “host cell” includes not only prokaryotes, such as E. coli, but also eukaryotes, such as fungal, insect, plant and animal cells. Animal cells include, for example, COS cells and HeLa cells. Fungal cells include yeast cells, such as Saccharomyces cereviseae cells. A host cell can be transformed or transfected with a DNA molecule (e.g., a vector) using techniques known to those of ordinary skill in this art, such as calcium phosphate or lithium acetate precipitation, electroporation, lipofection and particle bombardment. Host cells containing a vector can be used for such purposes as propagating the vector, producing a nucleic acid (e.g., DNA or interfering RNA) or expressing a polypeptide or fragments thereof.

Plants

Among the eukaryotic organisms featured in the invention are plants containing a recombinant nucleic acid construct described herein, e.g., a cytosine DNA methyltransferase coding sequence or interfering RNA sequence operably linked to a male gametophyte-specific regulatory element or a female gametophyte-specific regulatory element.

Plants useful as parents in the methods described above can be heterozygous or homozygous for a recombinant construct. However, when the nucleic acid construct encodes a cytosine DNA methyltransferase polypeptide, the use of plants homozygous for the construct can result in an alteration in a seed phenotype that is of greater magnitude that the alteration obtained when heterozygous plants are used. On the other hand, when the nucleic acid construct encodes a nucleic acid such as an antisense sequence, an interfering RNA sequence, or a ribozyme, plants that are heterozygous can often result in seed phenotype alterations that are as great as those observed with homozygous plants.

In another aspect, the invention feature a method of making a plant comprising introducing a recombinant nucleic acid construct into a plant cell. Techniques for introducing exogenous nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,204,253 and 6,013,863. If a cell or tissue culture is used as the recipient tissue for transformation, plants can be regenerated from transformed cultures by techniques known to those skilled in the art. Transgenic plants can be entered into a breeding program, e.g., to introduce a nucleic acid encoding a polypeptide into other lines, to transfer the nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, and subsequent generation plants, or seeds formed on BC₁, BC₂, BC₃, and subsequent generation plants. Seeds produced by a transgenic plant can be grown and then selfed (or outcrossed and selfed) to obtain seeds homozygous for the recombinant nucleic acid construct.

A suitable group of plants with which to practice the invention include dicots, such as safflower, alfalfa, soybean, rapeseed (high erucic acid and canola), or sunflower. Also suitable are monocots such as corn, wheat, rye, barley, oat, rice, millet, amaranth or sorghum. Also suitable are vegetable crops or root crops such as potato, watermelon, broccoli, peas, sweet corn, popcorn, tomato, beans (including kidney beans, lima beans, dry beans, green beans) and the like. Also suitable are fruit crops such as peach, pear, apple, cherry, orange, lemon, grapefruit, plum, mango and palm. Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Eschscholzia, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panicum, Pannesetum, Papaver, Persea, Phaseolus, Pinus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna and Zea. Also suitable are cells and tissues grown in liquid media or on semi-solid media.

The ability to alter a plant seed phenotype, e.g., increasing or decreasing seed weight, can provide advantages to agricultural producers and to consumers. For example, an increase in mean seed weight can result in increased overall yield or harvest index from a harvested crop, thereby providing an economic benefit to farmers. Moreover, an increase in mean seed weight can result in greater harvest of a specialty seed component per square acre, thereby providing greater land use efficiency. Exemplary specialty seed components include pharmaceuticals, alkaloids, terpenoids, antibodies, specialty starches, specialty oils, specialty proteins, and nutraceuticals such as sterols. Conversely, use of methods disclosed herein to achieve a decrease in mean seed weight can result in fruit or vegetable crops that, because of smaller seeds, are preferred by consumers.

Seed Compositions

In another aspect, the invention features a plant seed composition that contains seeds of at least two types. The two types can be populations (e.g., a synthetic population), lines, inbreds, hybrids, or commercial varieties. A synthetic population is a group of individual plants whose members are progeny of a multi-parental mating scheme, such that the group as a whole represents the allele frequencies of all parents. See, e.g, U.S. Pat. No. 6,320,106. The proportion of each type in a composition is measured as the number of seeds of a particular type divided by the total number of seeds in the composition, and can be formulated as desired to meet requirements based on geographic location, desired maturity and the like. The proportion of the first type can be from about 80 percent to about 99.9 percent, e.g., 80%, 85%, 90%, 91%, 92%, 93%, 94%, 95%, 96%, 97%, 98%, or 99%. The proportion of the second type can be from about 0.1 percent to about 20 percent, e.g., 0.5%, 1%, 2%, 3%, 4%, or 5%. If a third type is present in the composition, the proportion of the third type can be from about 0.1 percent to about 5 percent, e.g., 0.5%, 1%, 2%, 3%, 4%, or 5%. When large quantities of a seed composition are formulated, or when the same composition is formulated repeatedly, there may be some variation in the proportion of each type in the sample. Sampling error is known from statistics. In the present invention, such sampling error typically is about ±5% of the expected proportion, e.g., 90%±4.5%, or 5%±0.25%. A seed composition can be formulated in a quantity of about 35 kilograms (kg) or more, about 100 kg or more, about 1,000 kg or more, about 10,000 kg or more, or about 50,000 kg or more. In some embodiments, a plant seed composition further comprises additional types, e.g., about 0.1 to about 5 percent seeds of a third type.

Plants grown from seeds of the first type can overexpress a cytosine DNA methyltransferase in male gametophyte cells. Plants grown from seeds of the second type may or may not have a recombinant nucleic acid construct that inhibits expression of a cytosine DNA methyltransferase in female gametophyte cells.

For example, a seed composition of the invention can be made from two corn hybrids. A first corn hybrid can constitute 90% of the seeds in the composition and have a construct comprising a female gametophyte tissue-specific regulatory element operably linked to a nucleic acid sequence effective for reducing levels of global cytosine DNA methylation. The first corn hybrid can be male sterile if desired. A second corn hybrid can constitute 10% of the seed in the composition and have a construct that expresses a cytosine DNA methyltransferase in male gametophytic tissue. Alternatively, one of the two hybrids does not contain a nucleic acid construct described herein. Upon growing one of these compositions, pollen from the second hybrid will pollinate ears of the first hybrid, resulting in an increase in seed weight in the harvested crop for all plants of the composition. Other techniques for preparing and growing two seed types are described in U.S. Pat. No. 5,004,864 and these techniques and modifications thereof can be adapted for the methods describe herein. See also, U.S. Pat. No. 5,706,603.

Typically, a substantially uniform mixture of seeds of each of the types is conditioned and bagged in packaging material by means known in the art to form an article of manufacture. Such a bag of seed preferably has a package label accompanying the bag, e.g., a tag or label secured to the packaging material, a label printed on the packaging material or a label inserted within the bag. The package label indicates that the seeds therein are a mixture of types, e.g., two different types. The package label may indicate that plants grown from such seeds produce a harvested crop having increased seed weight relative to corresponding control plants.

Types in a seed composition of the invention typically have the same or very similar maturity, i.e., the same or very similar number of days from germination to crop seed maturation. In some embodiments, however, one or more types in a seed composition of the invention can have a different relative maturity compared to other types in the composition, i.e., the number of days from germination to mature seed for one type in a composition is statistically significantly different from that of another type in the composition.

The invention is further described in the following examples, which do not limit the scope of the invention.

### EXAMPLES

### Example 1

### Antisense Arabidopsis Methyltransferase Construct

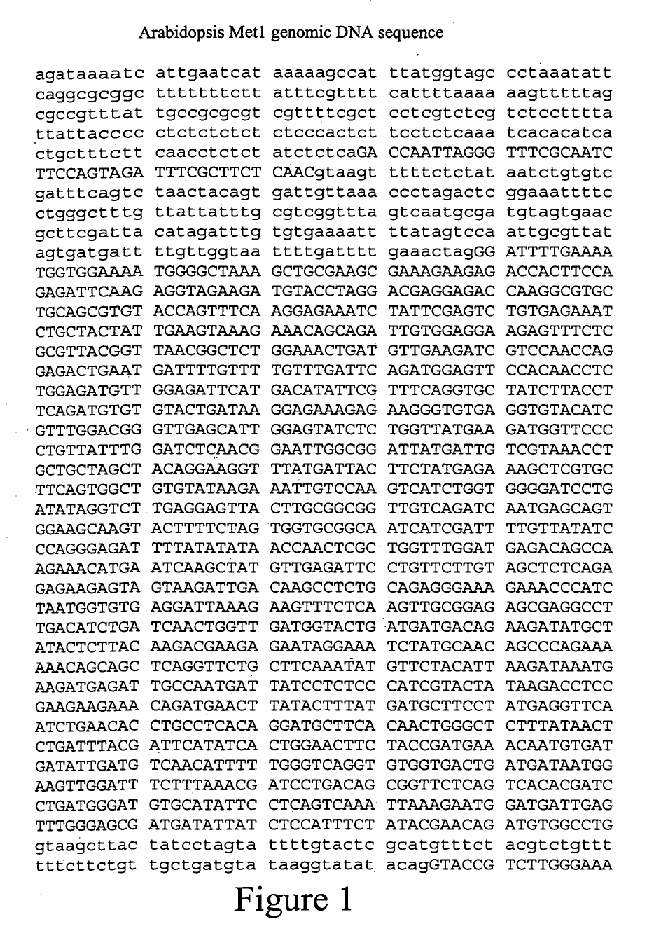

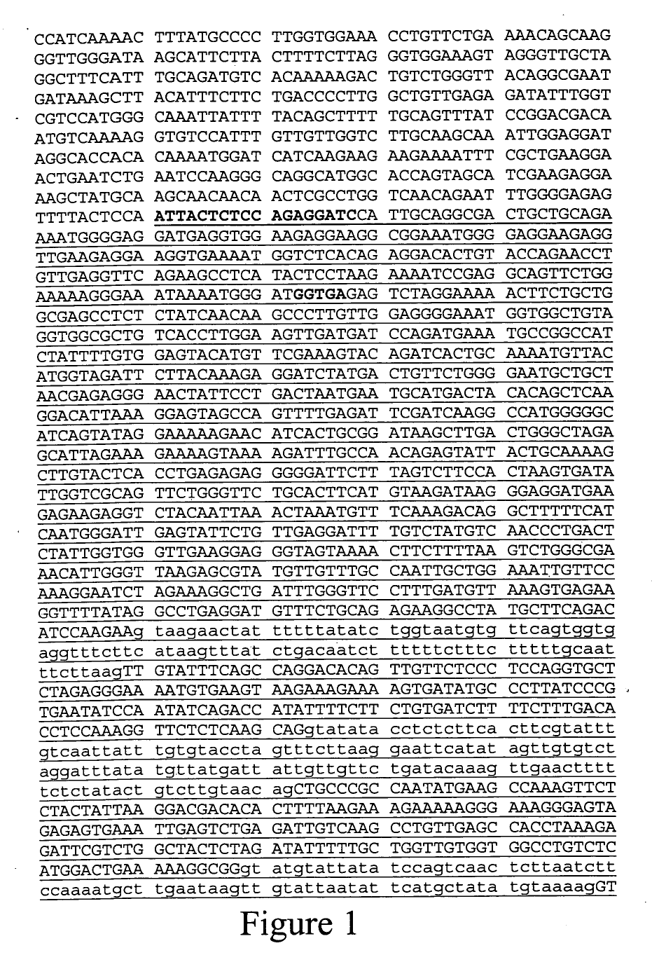

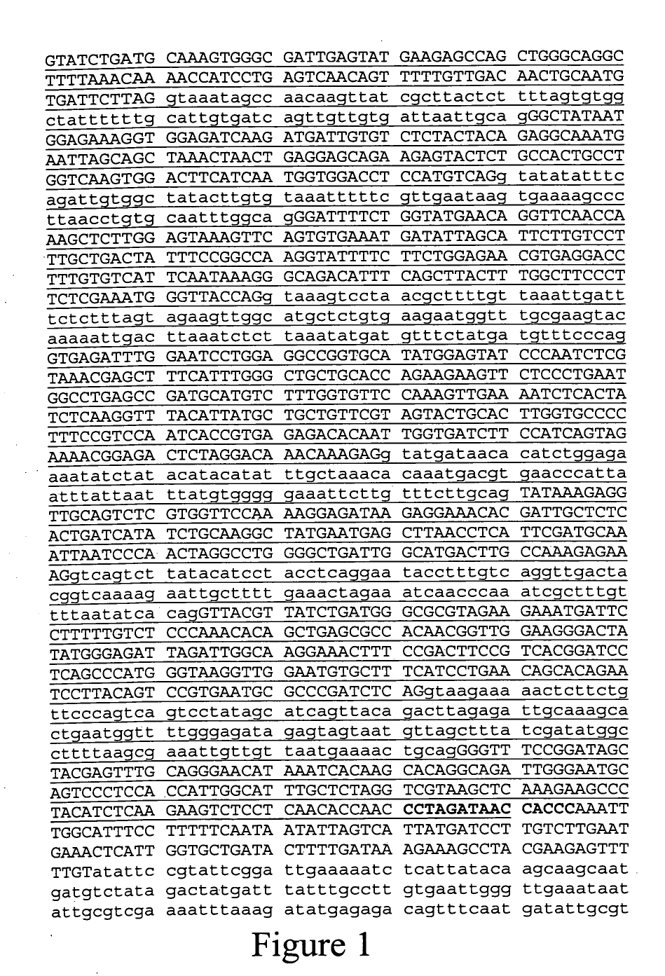

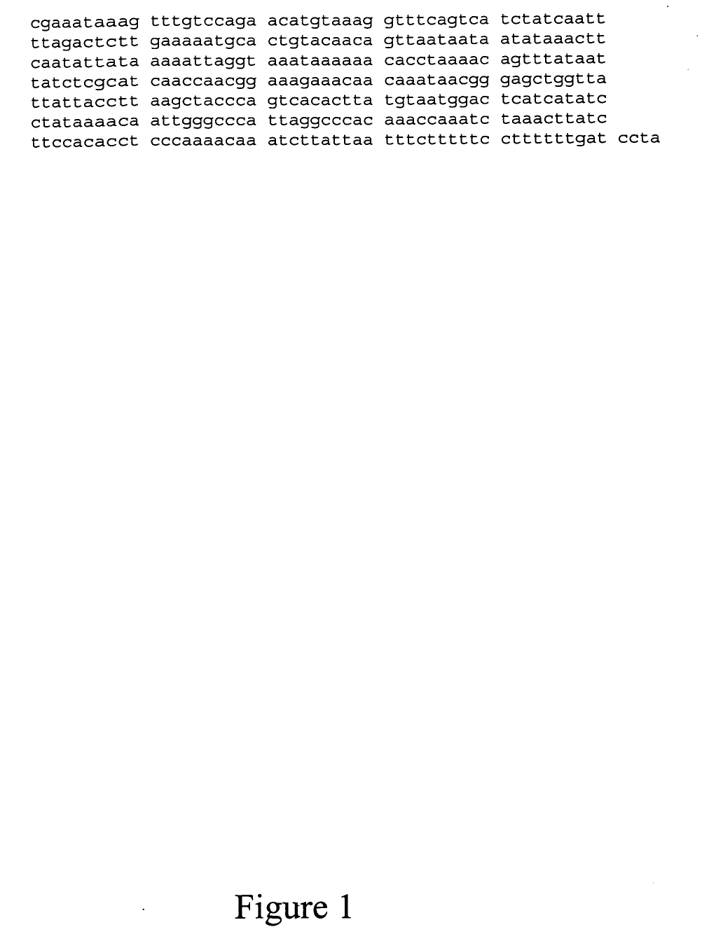

An antisense nucleic acid to the Arabidopsis Met1 cytosine DNA methyltransferase genomic sequence was prepared, based on the underlined portion of the Arabidopsis genomic DNA sequence shown in FIG. 1. The antisense nucleic acid is about 2.7 kb in length; its sequence is shown in the Sequence Listing.

A Met1 antisense nucleic acid construct was made using a vector containing left and right Agrobacterium T-DNA borders. The 2.7 kb Met1 antisense fragment was operably linked to a FIE-derived promoter driving transcription preferentially in female gametophytic tissue during embryo sac development, and inserted between the T-DNA borders. The sequence of the promoter is shown in SEQ ID NO: 5. See also, US Patent Publication 20030126642. The promoter facilitated expression in polar nuclei, the central cell and the early part of endosperm development, but did not drive detectable expression in the egg cell, zygote or male gametophyte tissue. The antisense fragment was also operably linked to a nos 3′ termination sequence. The construct, designated pRP:Met1a/s, also contained a bar selectable marker gene between the left and right T-DNA borders.

### Example 2

### Analysis of Transgenic Plants Containing an Arabidopsis Methyltransferase Antisense Construct

The following symbols are used in the Examples unless otherwise indicated: T1: first generation transformant; T2: second generation, progeny of self-pollinated T1 plants; T3: third generation, progeny of self-pollinated T2 plants; T4: fourth generation, progeny of self-pollinated T3 plants.

The pRP:Met1a/s antisense construct of Example 1 was introduced into Arabidopsis Columbia by the floral dip method essentially as described in Bechtold, N. et al., C.R. Acad. Sci. Paris, 316:1194-1199 (1993). Twenty-three independent transformants were recovered. T1 seeds were germinated and allowed to self-pollinate. In 14 of the transformants, T2 seeds were wild type in size, with aborted ovules in some or many of the siliques. In one of these 14 transformants, some of the T2 seeds were white.

In 9 of the transformants, T2 seeds were either wild type in size, or larger in size. Some siliques had aborted seeds. A sample of T2 seeds from each of these 9 transformants was germinated and analyzed for the presence of the pRP:Met1a/s construct by PCR analysis. Eight of the 9 transformants were found to segregate for the pRP:Met1a/s construct in the expected 3:1 ratio, indicating insertion of the construct at a single locus. The single locus transformants were grown to maturity and allowed to self-pollinate. Three replicates of 200 T3 seeds from each of the 8 transformants were weighed. The average T3 seed weight for 5 of the 8 transformants was higher than the average seed weight for wild-type Columbia plants.

T3 seeds from the 8 single locus transformants were germinated and the resulting plants were allowed to self-pollinate. Siliques on T3 plants were measured and mature T4 seeds were collected and measured. The results for ten homozygous T3 plants derived from T2 plant #23 and T1 transformation event #34, are shown in Table 1, as well as the results for five homozygous T3 plants derived from T2 plant #20 and T1 transformation event #34.

The results for ten homozygous T3 plants, derived from T2 plant #23 and T1 transformation event #32, are shown in Table 2, as wells as the results for five homozygous T3 plants, derived from T2 plant #13 and T1 transformation event #32.

TABLE 1
Analysis of T4 Seeds from Two T3 Homozygotes of Event #34

| Phenotype | Wild-type (Col) | #23 (10 plants) | #20 (5 Plants) |
|---|---|---|---|
| Mean Seed | 23.00 ± 0.273 | 26.47 ± 0.498 | 26.88 ± 0.412 |
| Weight ± SE | (n = 10) | (n = 10) | (n = 5) |
| (ug/seed) | | | |
| Minimum Seed | 21.52 | 24.62 | 25.93 |
| Weight | | | |
| Maximum Seed | 23.97 | 29.07 | 28.03 |
| Weight | | | |
| P-value (seed | — | 2.218E−05 | 3.055E−05 |
| weight) | | | |
| Silique Length ± SE | 14.3 ± 0.13 | 14.5 ± 0.12 | 14.9 ± 0.19 |
| (mm) | (n = 30) | (n = 30) | (n = 15) |
| Visible Seed | 57.4 ± 0.92 | 52.5 ± 0.95 | 56.4 ± 1.07 |
| No. per silique ± SE | (n = 30) | (n = 30) | (n = 15) |
| Aborted Seed | 0.6 ± 0.0.27 | 0.4 ± 0.18 | 0.0 ± 0.07 |
| No. per silique ± SE | (n = 30) | (n = 30) | (n = 15) |
| % of abortion | 0.9 ± 0.43% | 0.7 ± 0.34% | 0. ± 0.1% |

TABLE 2
Analysis of T4 Seeds from Two T3 Homozygotes of Event #32

| Phenotype | Wild-type (Col) | #23 (10 Plants) | #13 (5 Plants) |
|---|---|---|---|
| Mean Seed | 22.44 ± 0.180 | 25.99 ± 0.193 | 26.51 ± 0.429 |
| Weight ± SE | (n = 10) | (n = 10) | (n = 5) |
| (ug/seed) | | | |
| Minimum Seed | 21.28 | 25.10 | 25.33 |
| Weight | | | |
| Maximum Seed | 23.07 | 26.94 | 27.87 |
| Weight | | | |
| P-value (seed | | 8.14E−11 | 1.10E−07 |
| weight) | | | |
| Silique Length ± SE | 15.3 ± 0.22 | 15.9 ± 0.20 | 16.2 ± 0.24 |
| (mm) | (n = 30) | (n = 30) | (n = 15) |
| Visible Seed | 63.3 ± 1.52 | 61.6 ± 1.56 | 67.1 ± 1.56 |
| No. per silique ± SE | (n = 30) | (n = 30) | (n = 15) |
| Aborted Seed | 0.3 ± 0.30 | 0.2 ± 0.15 | 0.7 ± 0.33 |
| No. per silique ± SE | (n = 30) | (n = 30) | (n = 15) |
| % of abortion | 0.5 ± 0.50% | 0.3 ± 0.30% | 1.2 ± 0.53% |
| | (n = 30) | (n = 30) | (n = 15) |

The results showed that for progeny of event #34, average seed weight increased by 15.1% and 16.9%, respectively, in T4 generation seeds. The results showed that for progeny of event #32, average seed weight increased by 15.8% and 18.1%, respectively, in T4 generation seeds.

### Example 3

### Arabidopsis Methyltransferase Sense Construct

A nucleic acid containing a full-length Arabidopsis Met1 methyltransferase coding sequence was constructed. The nucleic acid was about 4.5 kb in length. A Met1 sense nucleic acid construct was made by operably linking the 4.5 kb Met1 nucleic acid in sense orientation to a promoter driving transcription preferentially in female gametophytic tissue during embryo sac development. The promoter facilitated expression in polar nuclei, the central cell and the early part of endosperm development, but did not drive detectable expression in the egg cell, zygote or male gametophyte tissue. The promoter also drove expression during the early part of endosperm development. The sense construct was designated pRP:Met1s.

### Example 4

### Analysis of Transgenic Plants Containing an Arabidopsis Methyltransferase Sense Construct

The pRP:Met1s construct of Example 3 was introduced into Arabidopsis Wassilewskija (WS) by the floral dip method essentially as described in Bechtold, N. et al., C.R. Acad. Sci. Paris, 316:1194-1199 (1993). Eleven independent transformants were recovered. The T1 transformants were grown and allowed to self-pollinate. Three of the transformants produced T2 siliques that had wild-type seeds, small seeds and some aborted ovules. T2 seeds from Event #1 were germinated and the resulting plants were allowed to self-pollinate. Siliques on T2 plants were measured and mature T3 seeds were collected and measured. Mature T3 seeds from one of the T1 transformants, Event #1, were observed into two classes, those appearing to have normal size and those appearing to have smaller size. Samples of both types of seeds were analyzed and the results are shown in Table 3.

TABLE 3
Analysis of T3 seeds from Event #1

| Weights of T3 | | | |
|---|---|---|---|
| Seeds of Event | | | |
| #1 | | | |
| Phenotype | Wild-type (Ws) | Class I | #1 Class II |
| Mean Seed | 20.33 ± 0.329 | 20.35 ± 0.297 | 13.75 ± 0.477 |
| Weight ± SE | (n = 5) | (n = 5) | (n = 5) |
| (ug/seed) | | | |
| Minimum Seed | 19.33 | 19.73 | 12.45 |
| Weight | | | |
| Maximum Seed | 21.38 | 21.38 | 15.10 |
| Weight | | | |
| P-value (seed | | 0.959 | 3.25202E−06 |
| weight) | | | |
| Silique Length ± SE | 15.5 ± 0.24 | 14.9 ± 0.35 | 11.5 ± 0.45 |
| (mm) | (n = 15) | (n = 15) | (n = 15) |
| Visible Seed | 60.1 ± 1.91 | 60.4 ± 2.62 | 47 ± 1.31 |
| No. per silique ± SE | (n = 15) | (n = 15) | (n = 15) |
| Aborted Seed | 2.3 ± 0.76 | 2.4 ± 0.71 | 0.6 ± 0.62 |
| No. per silique ± SE | (n = 15) | (n = 15) | (n = 15) |
| % of abortion | 1.9 ± 0.82% | 2.6 ± 1.17% | 1.5 ± 1.54% |
| | (n = 15) | (n = 15) | (n = 15) |

The results indicated that class II seeds had a mean weight that was 32.5% less than that of control W/S seeds.

### Example 5

### Arabidopsis Methyltransferase Antisense Construct

The 2.7 kb antisense nucleic acid of Example 1 was operably linked to an Arabidopsis DME promoter nucleic acid. The nucleotide sequence of the DME promoter is shown in Kinoshita et al., Proc. Natl. Acad. Sci. 98:14156-14161 (2001). The DME:Met1a/s construct was introduced into Arabidopsis cultivar WS as described in Bechtold, N. et al., C.R. Acad. Sci. Paris, 316:1194-1199 (1993). Mature T1 seeds were germinated and allowed to self-pollinate. Mature T2 seeds from independent transformants were observed to fall into two classes, those appearing to have normal size and those appearing to have a larger size. T2 seeds of each class are germinated and allowed to self-pollinate. T3 seeds are analyzed for mean seed weight and for the presence of the DME:Met1a/s transgene.

### Example 6

### Composition of Transgenic Arabidopsis Seeds

T3 seeds from homozygous plants described in Example 2 (#34-20 and #34-23) and T4 seeds from two progeny plants of #34-20 and #34-23 (#34-20-10, #34-20-13, #34-23-04 and #34-23-06) were collected. The levels of 82 compounds were measured in each batch of seeds, relative to the levels in non-transgenic T4 segregant seed collected from line #34-16-04. The compounds analyzed were: L-alanine, glycine, L-valine, L-leucine, L-isoleucine, L-serine, L-proline, L-threonine, homoserine, trans-4-L-hydroxyproline, L-aspartic acid, L-methionine, L-cysteine, L-glutamic acid, L-glutamine, L-phenylalanine, L-asparagine, L-ornithine, L-lysine, L-histidine, L-tryptophan, DL-lactic acid, glycolic acid, pyruvic acid, oxalic acid, phosphoric acid, glyceric acid, benzoic acid, fumaric acid, succinic acid, citramalic acid, malic acid, 2-hydroxybenzoic acid, ribonic acid-γ-lactone, α-ketoglutaric acid, quinic acid, shikimic acid, citric acid, isocitric acid, 3-phosphoglyceric acid, gluconic acid, xylose/arabinose, fucose, fructose, mannose, galactose, glucose, sucrose, maltose, trehalose, isomaltose, gycerol, ribitol, xylitol/arabitol, mannitol, inositol, maltitol, undecanoic acid, caprylic acid (C8:0), capric acid (C10:0), lauric acid (C12:0), myristic acid (C14:0), palmitic acid (C16:0), stearic acid (C18:0), oleic acid (C18:1), linoleic acid (C18:2), linolenic acid (C18:3), behenic acid (C22:0), lignoceric acid (C24:0), L-tetradecanol, hexadecanol, L-octadecanol, L-docosanol, L-octacosanol, L-triacontanol, squalene, cholesterol, stigmasterol, sitosterol and campesterol.

Extractions were done from each batch of seeds in duplicate or triplicate to generate replicate samples for GC-MS analysis. Examination of the data, normalized to an internal standard and to control levels, showed that the composition of seeds containing the pRP:Met1a/s construct was essentially indistinguishable from that of the control seeds for 80 out of the 82 compounds. T4 seeds from the #34-23-04, #34-23-06 and #34-20-10 plants had a reduction in linoleic acid and linolenic acid content relative to control seeds. T4 seeds from the #34-20-13 plants had a very slight reduction in linoleic acid and linolenic acid content relative to control seeds. No reduction in linoleic acid or linolenic acid was observed in the parental #34-23 or #34-20 T3 seeds.

### Example 7

### Analysis of Transgenic Plants Containing an Arabidopsis Methyltransferase RNAi Construct

An RNAi construct was made by operably linking a CaMV35S promoter to a sequence effective for being transcribed into an interfering RNA. The RNAi sequence comprised about 2.7 kb of the Arabidopsis Met1 sequence in sense orientation and an inverted repeat of a nos terminator sequence. The construct was made using standard molecular biology techniques. See, Brummell et al., Plant J., 33:793-800 (2004). The construct was inserted into a vector that contained a selectable marker gene conferring resistance to the herbicide Basta®.

The RNAi construct vector was introduced into Arabidopsis by the Agrobacterium-mediated method described in Example 2. Eight independent T1 plants were regenerated after selection for Basta® resistance, and the plants were allowed to self-pollinate. Vegetative tissue from the T1 plants was analyzed for the amount of endogenous Met1 transcript. As a control, an empty RNAi vector, in which the CaMV35S promoter was operably linked to the inverted nos terminator sequence was also introduced into Arabidopsis, and vegetative tissue from a control plant was analyzed at the same stage in development. The results showed that the level of endogenous transcript in the T1 plants ranged from 15% to 58% of the control amount.

### Example 8

### Analysis of Transgenic Plants Containing a Rice Methyltransferase RNAi Construct

The following symbols are used in this Example: T0: plant regenerated from transformed tissue culture; T1: first generation, progeny of self-pollinated T0 plants; T2: second generation, progeny of self-pollinated T1 plants; T3: third generation, progeny of self-pollinated T2 plants.

An RNAi construct was made by operably linking a CaMV35S promoter to a sequence effective for being transcribed into an interfering RNA. The RNAi sequence comprised about 600 nucleotides of a rice cytosine DNA methyltransferase sense strand (N-terminal region) and an inverted repeat of a nos terminator sequence. The construct was made using standard molecular biology techniques. The sequence of the 35S::rice Met::inverted nos construct is shown in SEQ ID NO: 1. The rice Met portion of the construct is shown in SEQ ID NO: 2. The construct was inserted into a vector that contained a selectable marker gene conferring resistance to the herbicide Basta®.

The RNAi construct vector was introduced into a tissue culture of the rice cultivar Kitaake by an Agrobacterium-mediated transformation protocol. T₀ plants from twelve independent events were regenerated from tissue selected for Basta® resistance and allowed to self-pollinate. Transformed tissue of the twelve events was analyzed for the amount of endogenous transcript present for the specific methyltransferase expected to be affected by the RNAi construct. As a control, a tissue culture sample from transgenic Kitaake T₀ tissue plants containing a vector having the 35S promoter linked to the inverted nos terminator but lacking the methyltransferase RNAi was analyzed at the same stage in development. The results showed that the level of endogenous transcript in the T₀ plants ranged from 2% to 53% of the control amount.

A second RNAi construct was made in the same manner except that a region of about 600 nucleotides of the rice methyltransferase C-terminal region was used. The sequence of the second construct is shown in SEQ ID NO: 3. The rice Met portion of the second construct is shown in SEQ ID NO: 4. The second RNAi construct is introduced into rice cultivar Kitaake by an Agrobacterium-mediated protocol.

A number of embodiments of the invention have been described. Nevertheless, it will be understood that various modifications may be made without departing from the spirit and scope of the invention. Accordingly, other embodiments are within the scope of the following claims.

1. A method for the production of seeds, comprising the step of permitting a first plant to pollinate a second plant, said first plant having a first recombinant nucleic acid construct comprising a male gametophyte tissue-specific regulatory element operably linked to a first nucleic acid sequence effective for increasing levels of cytosine DNA methylation, said second plant having a second recombinant nucleic acid construct comprising a female gametophyte tissue-specific regulatory element operably linked to a second nucleic acid sequence effective for reducing levels of cytosine DNA methylation, wherein seeds that develop on said second plant have a mean seed weight that is increased compared to the mean seed weight of seeds that develop on a corresponding second plant that lacks said second recombinant nucleic acid construct pollinated by a corresponding first plant that lacks said first recombinant nucleic acid construct.

2. The method of claim 1, wherein said first plant is an inbred, a hybrid, a heterogeneous population, or a synthetic population.

3. The method of claim 1, wherein said second plant is an inbred, a hybrid, a heterogeneous population, or a synthetic population.

4. The method of claim 1, wherein said first plant is heterozygous for said recombinant nucleic acid construct.

5. The method of claim 1, wherein said first plant is homozygous for said recombinant nucleic acid construct.

6. The method of claim 1, wherein said second plant is heterozygous for said recombinant nucleic acid construct.

7. The method of claim 1, wherein said second plant is homozygous for said recombinant nucleic acid construct.

8. The method of claim 1, wherein said first and second plants are dicotyledonous plants.

9. The method of claim 8, wherein said first nucleic acid sequence of said first recombinant nucleic acid construct encodes a cytosine DNA methyltransferase comprising a polypeptide region having the sequence set forth in SEQ ID NO: 50.

10. The method of claim 8, wherein said first nucleic acid sequence of said first recombinant nucleic acid construct encodes a cytosine DNA methyltransferase having 50% or greater sequence identity to one of the sequences set forth in SEQ ID NOS: 28, 30, 34, 36, 38, and 40.

11. The method of claim 8, wherein said second nucleic acid sequence of said second recombinant nucleic acid construct is transcribed into an interfering RNA.

12. The method of claim 8, wherein said second nucleic acid sequence of said second recombinant nucleic acid construct is transcribed into an antisense nucleic acid.

13. The method of claim 1, wherein said first and second plants are monocotyledonous plants.

14. The method of claim 13, wherein said first nucleic acid sequence of said first recombinant nucleic acid construct encodes a cytosine DNA methyltransferase having 50% or greater sequence identity to one of the amino acid sequences shown in SEQ ID NOS: 44 and 46.

15. The method of claim 14, wherein first nucleic acid sequence has 80% or greater sequence identity to one of the amino acid sequences shown in SEQ ID NOS: 44 and 46.

16. The method of claim 15, wherein first nucleic acid sequence has the amino acid sequence set forth in SEQ ID NO: 44.

17. The method of claim 15, wherein first nucleic acid sequence has the amino acid sequences set forth in SEQ ID NO: 46.

18. The method of claim 13, wherein said first and second plants are corn or rice plants.

19. The method of claim 1, wherein said male gametophyte tissue-specific regulatory element comprises the sequence set forth in SEQ ID NO: 8.

20. The method of claim 1, wherein seeds that develop on said pollinated plant have a mean seed weight that is at least 10% greater than the mean seed weight of seeds that develop on a corresponding second plant that lacks said second recombinant nucleic acid construct pollinated by a corresponding first plant that lacks said first recombinant nucleic acid construct.

21. The method of claim 20, wherein seeds that develop on said pollinated plant have a mean seed weight that is from about 10% to about 50% greater than the mean seed weight of seeds that develop on a corresponding second plant that lacks said second recombinant nucleic acid construct pollinated by a corresponding first plant that lacks said first recombinant nucleic acid construct.

22. A method for the production of seeds, comprising the step of permitting a first plant to pollinate a second plant, said first plant having a recombinant nucleic acid construct comprising a male gametophyte tissue-specific regulatory element operably linked to a first nucleic acid sequence effective for decreasing levels of cytosine DNA methylation, wherein seeds that develop on said second plant have a mean seed weight that is decreased compared to the mean seed weight of seeds that develop on a corresponding second plant pollinated by a corresponding first plant that lacks said recombinant nucleic acid construct.

23. A method for the production of seeds, comprising the step of permitting pollination of a plant having a recombinant nucleic acid construct comprising a female gametophyte tissue-specific regulatory element operably linked to a nucleic acid sequence effective for reducing levels of cytosine DNA methylation, said pollination occurring with pollen that lacks said recombinant nucleic acid construct, wherein seeds that develop on said plant have a mean seed weight that is increased compared to the mean seed weight of seeds that develop on a corresponding plant that lacks said recombinant nucleic acid construct pollinated by a plant that lacks said recombinant nucleic acid construct.

24. The method of claim 23, wherein said pollinated plant is a dicotyledonous plant.

25. The method of claim 24, wherein said regulatory element is a female gametophyte tissue-specific promoter selected from the group consisting of SEQ ID NOS: 6, 25, and 22.

26. The method of claim 24, wherein said nucleic acid sequence effective for reducing levels of cytosine DNA methylation is transcribed into an interfering RNA.

27. The method of claim 26, wherein said nucleic acid sequence has a length of from 10 nucleotides to 4,500 nucleotides and 70% or greater sequence identity to one of the nucleic acid sequences set forth in SEQ ID NOS: 29, 31, 33, 35, 37, 39, 41, or complements thereof.

28. The method of claim 27, wherein said nucleic acid has a length of from 20 nucleotides to 1,000 nucleotides and 80% or greater sequence identity to one of the nucleic acid sequences set forth in SEQ ID NOS: 29, 31, 33, 35, 37, 39, 41, or complements thereof.

29. The method of claim 23, wherein said nucleic acid sequence effective for reducing levels of cytosine DNA methylation is transcribed into an antisense nucleic acid.

30. The method of claim 23, wherein said pollinated plant is a monocotyledonous plant.

31. The method of claim 30, wherein said nucleic acid sequence effective for reducing levels of cytosine DNA methylation is transcribed into an interfering RNA.

32. The method of claim 31, wherein said nucleic acid sequence has a length of from 10 nucleotides to 4,500 nucleotides and 70% or greater sequence identity to one of the nucleic acid sequences set forth in SEQ ID NOS: 43, 45, 47, 49, or complements thereof.

33. The method of claim 32, wherein said nucleic acid has a length of from 20 nucleotides to 1,000 nucleotides and 80% or greater sequence identity one of the nucleic acid sequences set forth in SEQ ID NOS: 43, 45, 47, 49, or complements thereof.

34. The method of claim 30, wherein said nucleic acid sequence effective for reducing levels of cytosine DNA methylation is transcribed into an antisense nucleic acid.

35. The method of claim 23, wherein said pollination occurs with pollen from a non-transgenic plant.

36. A method for the production of seeds, comprising the step of permitting pollination of a plant having a recombinant nucleic acid construct comprising a female gametophyte tissue-specific regulatory element operably linked to a nucleic acid sequence effective for increasing levels of cytosine DNA methylation, said pollination occurring with pollen that lacks said recombinant nucleic acid construct, wherein seeds that develop on said plant have a mean seed weight that is decreased compared to the mean seed weight of seeds that develop on a corresponding plant that lacks said recombinant nucleic acid construct pollinated by a plant that lacks said recombinant nucleic acid construct.

37. A method for the production of seeds, comprising the step of permitting a first plant to pollinate a second plant, said first plant having a recombinant nucleic acid construct comprising a male gametophyte tissue-specific regulatory element operably linked to a nucleic acid sequence effective for increasing levels of cytosine DNA methylation, wherein seeds that develop on said second plant have a mean seed weight that is increased compared to the mean seed weight of seeds that develop on a corresponding plant pollinated by a plant that lacks or does not express said recombinant nucleic acid construct.

38. The method of claim 37, wherein said first and second plants are dicotyledonous plants.

39. The method of claim 38, wherein said nucleic acid sequence effective for increasing levels of cytosine DNA methylation encodes a cytosine DNA methyltransferase comprising a polypeptide region having the amino acid sequence shown in SEQ ID NO: 50.

40. The method of claim 37, wherein said male gametophyte tissue-specific regulatory element is the SEQ ID NO: 8. Arabidopsis YP0180 promoter

41. The method of claim 37, wherein said first and second plants are monocotyledonous plants.

42. The method of claim 41, wherein said nucleic acid sequence encodes a cytosine DNA methyltransferase having 50% or greater sequence identity to one of the amino acid sequences shown in SEQ ID NO: 44 and SEQ ID NO: 46.

43. The method of claim 37, wherein seeds that develop on said pollinated plant have a mean seed weight that is at least 10% greater than the mean seed weight of seeds that develop on said corresponding plant that lacks said recombinant nucleic acid construct.

44. The method of claim 43, wherein seeds that develop on said pollinated plant have a mean seed weight that is from about 10% to about 50% greater than the mean seed weight of seeds that develop on said corresponding plant that lacks said recombinant nucleic acid construct.

45. A method for the production of seeds, comprising the step of permitting pollination among a plurality of plants that comprise a plurality of first plants, each of said first plants having a first recombinant nucleic acid construct comprising a male gametophyte tissue-specific regulatory element operably linked to a nucleic acid sequence effective for increasing levels of cytosine DNA methylation, wherein seeds that develop on said first plants after pollination have a mean seed weight that is increased compared to the mean seed weight of seeds that develop on corresponding plants that lack said recombinant nucleic acid construct.

46. The method of claim 45, wherein said pollination is predominantly self-pollination.

47. The method of claim 45, wherein said plurality of first plants are dicotyledonous plants.

48. The method of claim 45, wherein said plurality of plants further comprises a plurality of second plants, said second plants having a second recombinant nucleic acid construct comprising a female gametophyte tissue-specific regulatory element operably linked to a nucleic acid sequence effective for reducing levels of cytosine DNA methylation, and wherein seeds that develop on said second plants after pollination have a mean seed weight that is increased compared to the mean seed weight of seeds that develop on corresponding plants that lack said recombinant nucleic acid construct.

49. The method of claim 48, wherein said first and second plants are monocotyledonous plants.

50. The method of claim 49, wherein said plurality of plants further comprises a plurality of second plants, said second plants having a recombinant nucleic acid construct comprising a female gametophyte tissue-specific regulatory element operably linked to a nucleic acid sequence effective for reducing levels of cytosine DNA methylation, and wherein seeds that develop on said second plants after pollination have a mean seed weight that is increased compared to the mean seed weight of seeds that develop on corresponding plants that lack said recombinant nucleic acid construct.

51. The method of claim 45, wherein seeds that develop on said pollinated plants have a mean seed weight that is at least 10% greater than the mean seed weight of seeds that develop on said corresponding plants that lack said recombinant nucleic acid construct.

52. The method of claim 51, wherein seeds that develop on said pollinated plants have a mean seed weight that is from about 10% to about 50% greater than the mean seed weight of seeds that develop on said corresponding plants that lack said recombinant nucleic acid construct.

53. A transgenic host cell comprising a recombinant nucleic acid construct comprising a nucleic acid sequence effective for reducing levels of cytosine DNA methylation, said nucleic acid sequence operably linked to one or more regulatory elements that confer transcription in plant female gametophyte cell types.

54. The host cell of claim 53, wherein said one or more regulatory elements comprises one of the sequences set forth in SEQ ID NOS: 6, 22, and 25.

55. A transgenic host cell comprising a recombinant nucleic acid construct comprising a nucleic acid sequence effective for reducing levels of cytosine DNA methylation, said nucleic acid sequence operably linked to one or more regulatory elements that confer transcription in plant male gametophyte cell types.

56. The host cell of claim 55, wherein said one or more regulatory elements comprises the sequence set forth in SEQ ID NO: 8.

57. A transgenic plant comprising a recombinant nucleic acid construct comprising a nucleic acid sequence effective for reducing levels of cytosine DNA methylation, said nucleic acid sequence operably linked to one or more regulatory elements that confer transcription in female gametophyte cell types.

58. The plant of claim 57, wherein said one or more regulatory elements confer preferential transcription in polar cell nuclei and central cells relative to egg cells, zygotes and embryos.

59. The plant of claim 57, wherein said one or more regulatory elements comprises a sequence selected from SEQ ID NOS: 6-27.

60. The plant of claim 57, wherein said plant is a dicotyledonous plant.

61. The plant of claim 60, wherein said nucleic acid sequence effective for reducing levels of cytosine DNA methylation is transcribed into an interfering RNA.

62. The plant of claim 61, wherein said nucleic acid sequence has a length of from 10 nucleotides to 4,500 nucleotides and 70% or greater sequence identity to one of the nucleic acid sequences set forth in SEQ ID NOS: 29, 31, 33, 35, 37, 39 and 41, or complements thereof.

63. The plant of claim 62, wherein said nucleic acid has a length of from 20 nucleotides to 1,000 nucleotides and 80% or greater sequence identity to one of the nucleic acid sequences set forth in SEQ ID NOS: 29, 31, 33, 35, 37, 39, 41, or complements thereof.

64. The plant of claim 60, wherein said nucleic acid sequence effective for reducing levels of cytosine DNA methylation is transcribed into an antisense nucleic acid.

65. The plant of claim 57, wherein said plant is a monocotyledonous plant.

66. The plant of claim 65, wherein said nucleic acid sequence effective for reducing levels of cytosine DNA methylation is transcribed into an interfering RNA.

67. The plant of claim 66, wherein said nucleic acid sequence has a length of from 10 nucleotides to 4,500 nucleotides and 70% or greater sequence identity to one of the nucleic acid sequences set forth in SEQ ID NOS: 43, 45, 47, 49, or complements thereof.

68. The plant of claim 67, wherein said nucleic acid has a length of from 20 nucleotides to 1,000 nucleotides and 80% or greater sequence identity to one of the nucleic acid sequences set forth in SEQ ID NOS: 43, 45, 47, 49, or complements thereof.

69. The plant of claim 65, wherein said nucleic acid sequence effective for reducing levels of cytosine DNA methylation is transcribed into an antisense nucleic acid.

70. A transgenic plant comprising a recombinant nucleic acid construct comprising a nucleic acid sequence effective for reducing levels of cytosine DNA methylation, said nucleic acid sequence operably linked to one or more regulatory elements that confer transcription in male gametophyte cell types.

71. An article of manufacture comprising packaging material and at least a first type of seeds and a second type of seeds in said packaging material, wherein said seeds of said second type have a recombinant nucleic acid construct comprising a female gametophyte tissue-specific regulatory element operably linked to a nucleic acid sequence effective for reducing levels of cytosine DNA methylation.

72. The article of claim 71, wherein said first type of seeds are non-transgenic seeds.

73. The article of claim 71, wherein said seeds are corn seeds.
